(19) United States
(12) Patent Application Publication
(10) Pub. No.: US 2026/0207778 A1
(43) Pub. Date: Jul. 23, 2026

(54) Treatment of GEMIN5-Mediated Disorders

(71) Applicant: University of Pittsburgh - Of the Commonwealth System of Higher Education, Pittsburgh, PA (US)

(72) Inventors: Udai Pandey, Sewickley, PA (US); Tyler Fortuna, Pittsburgh, PA (US); Sukhleen Kour, Pittsburgh, PA (US); Deepa Soundara Rajan, Sewickley, PA (US)

(21) Appl. No.: 19/141,402

(22) PCT Filed: Dec. 21, 2023
(86) PCT No.: PCT/US23/85477

§ 371 Date: Jun. 20, 2025

Related U.S. Application Data
(60) Provisional application No. 63/477,022, filed on Dec. 23, 2022.

(51) Int. Cl. A61K 48/00; A61K 31/519; A61K 38/17; A61P 25/28; C12N 15/113; C12N 15/86

(52) U.S. Cl. CPC A61K 48/005; A61K 31/519; A61K 38/1709; A61K 48/0075; A61P 25/28; C12N 15/113; C12N 15/86; C12N 2310/11; C12N 2750/14143

(57) ABSTRACT

Provided herein is a method for treating a neurodevelopmental disorder in a patient, wherein the patient having the neurodevelopmental disorder comprises one or more biallelic variants in GEMIN5 and one or more of a developmental delay, cerebellar atrophy, ataxia, and/or hypotonia.

### CROSS REFERENCE TO RELATED APPLICATIONS

This application is the United States national phase of International Application No. PCT/US23/85477 filed Dec. 21, 2023, and claims priority to U.S. Provisional Patent Application No. 63/477,022 filed Dec. 23, 2023, the disclosures of which are hereby incorporated by reference in their entireties.

### REFERENCE TO A SEQUENCE LISTING

The Sequence Listing associated with this application is filed in electronic format via Patent Center and is hereby incorporated by reference into the specification in its entirety. The name of the XML file containing the Sequence Listing is 06527-2308518.xml. The size of the XML file is 45,572 bytes and the XML file was created on Dec. 20, 2023.

RNA-binding proteins (RBPs) regulate multiple molecular functions including splicing, localization, translation, and mRNA stability. RBPs exert those functions by forming large complexes with other proteins, such as small nuclear ribonuclear proteins (snRNPs). Perturbing the physiological functions of RBPs can lead to motor neuron diseases, such as amyotrophic lateral sclerosis and spinal muscular atrophy (SMA). The snRNPs, including survival motor neuron (SMN), GEMIN2, GEMIN3, GEMIN4, GEMIN5, GEMIN6, GEMIN7, GEMIN8, and Smith (Sm) core proteins, are important components of spliceosomes and help to remove introns from pre-mRNAs to generate mRNAs.

GEMIN5 is a highly conserved protein across vertebrates that is involved in the spliceosomal snRNPs biogenesis. GEMIN5 has been shown to localize in both the nucleus and the cytoplasm. GEMIN5 interacts with numerous SMN complex members but is directly associated with GEMIN2 and SMN. The initiator of the SMN complex is GEMIN5, as it specifically recognizes each unique snRNP code to begin the process of snRNP-core assembly. For example, in patients with SMA, disease severity is associated with the amount of functional SMN protein production, which correlates to the degree of snRNP assembly defects.

The majority of GEMIN5 variants cause loss-of-function by reducing GEMIN5 protein expression and disrupting snRNP-core complex dynamics (Kour et al. Nat Commun, 2021, Vol. 12, No. 1, p. 2558). The neurodevelopmental disorder identified by Kour et al. features developmental delay, cerebellar atrophy, and predominant motor dysfunction along with hypotonia and was identified as resulting from biallelic variants in the GEMIN5 gene (neurodevelopmental disorder with cerebellar atrophy and motor dysfunction (NEDCAM), OMIM #619333, see also UniProt Q8TEQ6 GEMI5_HUMAN, NCBI Gene ID: 25929, and NCBI Reference Sequences: NM_001252156.2 and NP_001239085.1). It would be beneficial to restore SMN expression in patients with this neurodevelopmental disorder in order to upregulate endogenous levels of GEMIN5 in a patient using a therapeutic agent, such as antisense oligonucleotides (ASO) and/or SMN gene replacement therapies.

### SUMMARY OF THE INVENTION

According to an aspect or embodiment of the invention, a method for treating a neurodevelopmental disorder, amyotrophic lateral sclerosis, or spinal muscular atrophy in a patient, comprising administering to the patient a therapeutic agent in an amount effective to treat the neurodevelopmental disorder,
wherein the therapeutic agent is chosen from one or more of the following:
   an antisense oligonucleotide (ASO) targeting SMN1 or SMN2, e.g. targeting intronic splicing silencer N1 (ISS-N1) within SMN2 intron 7;
   a mRNA splicing modifier, a pharmaceutically acceptable salt thereof, or a solvate thereof; or
a gene therapy for expressing an SMN1 or SMN2 transcript;

wherein when the patient has a neurodevelopmental disorder, the patient having the neurodevelopmental disorder comprises one or more biallelic variants in GEMIN5 and one or more of a developmental delay, motor dysfunction, cerebellar atrophy, ataxia, and/or hypotonia (e.g., NEDDCAM).

According to another aspect or embodiment of the invention, provided herein is a A method for treating a neurodevelopmental disorder, amyotrophic lateral sclerosis, or spinal muscular atrophy in a patient, comprising administering to the patient a therapeutic agent in an amount effective to treat the neurodevelopmental disorder,
wherein the therapeutic agent is chosen from one or more of the following:
   an antisense oligonucleotide (ASO) targeting SMN1 or SMN2, e.g. targeting intronic splicing silencer N1 (ISS-N1) within SMN2 intron 7;
   a mRNA splicing modifier, a pharmaceutically acceptable salt thereof, or a solvate thereof; or
a gene therapy for expressing an SMN1 or SMN2 transcript;

wherein when the patient has a neurodevelopmental disorder, the patient having the neurodevelopmental disorder comprises one or more biallelic variants in GEMIN5 and one or more of a developmental delay, motor dysfunction, cerebellar atrophy, ataxia, and/or hypotonia (e.g., NEDDCAM).

The following clauses outline additional aspects, embodiments, and/or examples of the present invention.

Clause 1. A method for treating a neurodevelopmental disorder, amyotrophic lateral sclerosis, or spinal muscular atrophy in a patient, comprising administering to the patient a therapeutic agent in an amount effective to treat the neurodevelopmental disorder,
wherein the therapeutic agent is chosen from one or more of the following:
   an antisense oligonucleotide (ASO) targeting SMN1 or SMN2, e.g. targeting intronic splicing silencer N1 (ISS-N1) within SMN2 intron 7;
   a mRNA splicing modifier, a pharmaceutically acceptable salt thereof, or a solvate thereof; or
a gene therapy for expressing an SMN1 or SMN2 transcript;
wherein when the patient has a neurodevelopmental disorder, the patient having the neurodevelopmental disorder comprises one or more biallelic variants in GEMIN5 and one or more of a developmental delay, motor dysfunction, cerebellar atrophy, ataxia, and/or hypotonia (e.g., NEDDCAM).

Clause 2. The method of clause 1, wherein the patient comprises one or more biallelic variants in GEMIN5 and wherein the biallelic variant is, relative to SEQ ID NO: 2: p. (Leu1068Pro), p. (Ala1007Thr), p. (His923Pro), p. (His913Arg), p. (Asp704Glu), p. (Gly683Asp), p. (Ser1000Pro) p. (Tyr1282His), p. (Cys1205Trp), p. (Arg1014Gln), p. (Arg1016Cys), p. (Met485Hfs*27), p. (Arg1016Cys), p. (Arg899Pfs*3), p. (Trp373*), p. (Ala994Val), p. (Pro594Arg), p. (Ser543Gly), or combinations thereof.

Clause 3. The method of clause 1, wherein the patient has a developmental delay, motor dysfunction, cerebellar atrophy, and ataxia.

Clause 4. The method of clause 1, wherein the administration of the ASO, the mRNA splicing modifier, the gene therapy, or combinations thereof upregulates survival motor neuron (SMN) protein.

Clause 5. The method of clause 5, wherein the upregulation of the SMN protein increases the expression of GEMIN5 protein.

Clause 6. The method of clause 1, wherein the therapeutic agent is an ASO an antisense oligonucleotide (ASO) targeting SMN1 or SMN2, e.g. targeting intronic splicing silencer N1 (ISS-N1) within SMN2 intron 7.

Clause 7. The method of clause 6, wherein the ASO is administered parenterally, e.g., intrathecally.

Clause 8. The method of clause 6, wherein the ASO is nusinersen.

Clause 9. The method of clause 8, wherein from at least 0.5 mg/mL to at least 3 mg/mL of nusinersen, or a pharmaceutically-acceptable salt or solvate thereof, is administered to the patient in multiples doses, such as every 4 months.

Clause 10. The method of clause 1, wherein the therapeutic agent is an mRNA splicing modifier, a pharmaceutically acceptable salt thereof, or a solvate thereof.

Clause 11. The method of clause 10, wherein the mRNA splicing modifier is an SMN2 mRNA splicing modifier.

Clause 12. The method of clause 11, wherein the SMN2 mRNA splicing modifier comprises a compound having the formula (I):

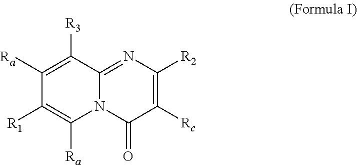

or a free acid, free base, a chloride salt, hydrobromide salt, hydrochloride salt, dihydrochloride salt, acetate salt, trifluoroacetate salt, trifluoroacetic acid salt, isotopologue, stereoisomer, racemate, enantiomer, diastereomer or tautomer thereof, wherein:

R₁ is a heterocyclyl, e.g., azetidinyl, tetrahydrofuranyl, pyrrolidinyl, piperidinyl, piperazinyl, 1,4-diazepanyl, 1,2,5,6-tetrahydropyridinyl, 1,2,3,6-tetrahydropyridinyl, hexahydropyrrolo[3,4-b]pyrrol-(1H)-yl, (3aS,6aS)-hexahydropyrrolo[3,4-b]pyrrol-(1H)-yl, (3aR,6aR)-hexahydropyrrolo[3,4-b]pyrrol-(1H)-yl, hexahydropyrrolo[3,4-b]pyrrol-(2H)-yl, (3aS,6aS)-hexahydropyrrolo[3,4-b]pyrrol-(2H)-yl, hexahydropyrrolo[3,4-c]pyrrol-(1H)-yl, (3aR,6aS)-hexahydropyrrolo[3,4-c]pyrrol-(1H)-yl, octahydro-5H-pyrrolo[3,2-c]pyridinyl, octahydro-6H-pyrrolo[3,4-b]pyridinyl, (4aR,7aR)-octahydro-6H-pyrrolo[3,4-b]pyridinyl, (4aS, 7aS)-octahydro-6H-pyrrolo[3,4-b]pyridinyl, hexahydropyrrolo[1,2-a]pyrazin-(2H)-one, hexahydropyrrolo[1,2-a]pyrazin-(1H)-yl, (7R,8aS)-hexahydropyrrolo[1,2-a]pyrazin-(1H)-yl, (8aS)-hexahydropyrrolo[1,2-a]pyrazin-(1H)-yl, (8aR)-hexahydropyrrolo[1,2-a]pyrazin-(1H)-yl, (8aS)-octahydropyrrolo[1,2-a]pyrazin-(1H)-yl, (8aR)-octahydropyrrolo[1,2-a]pyrazin-(1H)-yl, octahydro-2H-pyrido[1,2-a]pyrazinyl, 3-azabicyclo[3.1.0]hexyl, (1R,5S)-3-azabicyclo[3.1.0]hexyl, 8-azabicyclo[3.2.1]octyl, (1R,5S)-8-azabicyclo[3.2.1]octyl, 8-azabicyclo[3.2.1]oct-2-enyl, (1R,5S)-8-azabicyclo[3.2.1]oct-2-enyl, 9-azabicyclo[3.3.1]nonyl, (1R,5S)-9-azabicyclo[3.3.1]nonyl, 2,5-diazabicyclo[2.2.1]heptyl, (1S,4S)-2,5-diazabicyclo[2.2.1]heptyl, 2,5-diazabicyclo[2.2.2]octyl, 3,8-diazabicyclo[3.2.1]octyl, (1R,5S)-3,8-diazabicyclo[3.2.1]octyl, 1,4-diazabicyclo[3.2.2]nonyl, azaspiro[3.3]heptyl, 2,6 diazaspiro[3.3]heptyl, 2,7-diazaspiro[3.5]nonyl, 5,8-diazaspiro[3.5]nonyl, 2,7-diazaspiro[4.4]nonyl and 6,9-diazaspiro[4.5]decyl, wherein, the heterocyclyl is optionally substituted with one, two or three R₃ substituents and optionally, with one additional R₄ substituent; or, wherein, heterocyclyl is optionally substituted with one, two, three or four R₃ substituents, where R₃ is, in each instance, independently selected from cyano, halogen, hydroxy, oxo, C₁₋₈alkyl, e.g. methyl, ethyl, propyl, isopropyl and tert-butyl, halo-C₁₋₈alkyl, C₁₋₈ alkyl-carbonyl, C₁₋₈alkoxy, halo-C₁₋₈alkoxy, C₁₋₈alkoxy-C₁₋₈alkyl, C₁₋₈alkoxy-carbonyl, amino, C₁₋₈alkyl-amino, (C₁₋₈alkyl)₂-amino, amino-C₁₋₈alkyl, C₁₋₈alkyl-amino-C₁₋₈alkyl, (C₁₋₈alkyl)₂-amino-C₁₋₈alkyl, amino-C₁₋₈alkyl-amino, C₁₋₈alkyl-amino-C₁₋₈alkyl-amino, (C₁-salkyl-amino-C ₁₋₈alkyl)₂-amino, (C₁₋₈alkyl)₂-amino-C₁₋₈alkyl-amino, [(C₁₋₈alkyl)₂-amino-C₁-8alkyl]₂-amino, (C₁₋₈alkyl-amino-C₁₋₈alkyl) (C₁₋₈alkyl)amino, [(C₁₋₈alkyl)₂-amino-C₁-8alkyl](C₁₋₈alkyl)amino, C₁₋₈alkoxy-C₁₋₈alkyl-amino, (C₁₋₈alkoxy-C₁₋₈alkyl)₂-amino, (C₁₋₈alkoxy-C₁₋₈alkyl) (C₁₋₈alkyl)amino, C₁-galkyl-carbonyl-amino, C₁₋₈alkoxy-carbonyl-amino, hydroxy-C₁₋₈alkyl, hydroxy-C₁₋₈alkoxy-C₁₋₈alkyl, hydroxy-C₁₋₈alkyl-amino, (hydroxy-C₁₋₈alkyl)₂-amino or (hydroxy-C₁₋₈alkyl) (C₁₋₈alkyl)amino; and R⁴ is C₃₋₁₄cycloalkyl, C₃₋₁₄cycloalkyl-C₁₋₈alkyl, C₃₋₁₄cycloalkyl-amino, aryl-C₁₋₈alkyl, aryl-C₁₋₈alkoxy-carbonyl, aryl-sulfonyloxy-C₁₋₈ alkyl, heterocyclyl or heterocyclyl-C₁₋₈alkyl; wherein, each instance of C₃₋₁₄cycloalkyl, aryl and heterocyclyl is optionally substituted with one, two or three R₅ substituents, where R₅ is, in each instance, independently selected from halogen, hydroxy, cyano, nitro, C₁₋₈alkyl, halo-C₁₋₈alkyl, C₁₋₈alkoxy, halo-C₁₋₈alkoxy, amino, C₁₋₈alkyl-amino, (C₁₋₈alkyl)₂-amino or C₁₋₈alkyl-thio;
R₂ is heteroaryl; wherein the heteroaryl is optionally substituted with one, two or three R₆ substituents and optionally, with one additional R₇ substituent where R₆ is, in each instance, independently selected from halogen, hydroxy, cyano, nitro, C₁₋₈alkyl, such as methyl, ethyl, propyl, isopropyl and tert-butyl, C₂-galkenyl, halo-C₁₋₈alkyl, hydroxy-C₁₋₈alkyl, C₁₋₈alkoxy, halo-C₁₋₈alkoxy, C₁₋₈alkoxy-C₁₋₈alkyl, amino, C₁₋₈alkyl-amino, (C₁₋₈alkyl)₂-amino or C₁₋₈ alkyl-thio; and, R₇ is C₃₋₁₄cycloalkyl, C₃₋₁₄cycloalkyl-oxy, aryl, heterocyclyl or heteroaryl;
Rₐ is, in each instance, independently selected from hydrogen, halogen or C₁₋₈alkyl;
R_{b} is hydrogen, halogen, C₁-galkyl or C₁₋₈alkoxy; and
R_{c} is hydrogen, halogen or C₁₋₈alkyl.

Clause 13. The method of clause 11, wherein the SMN2 mRNA splicing modifier is risdiplam, e.g., 7-(4,7-diazaspiro[2.5]octan-7-yl)-2-(2,8-dimethylimidazo[1,2-b]pyridazin-6-yl) pyrido[1,2-a]pyrimidin-4-one, or a pharmaceutically-acceptable salt or solvate thereof.

Clause 14. The method of clause 13, wherein at least 0.15 milligrams per kilogram of body weight (mg/kg) or at least 5 milligrams of risdiplam, or a pharmaceutically-acceptable salt or solvate thereof, is administered daily to the patient.

Clause 15. The method of clause 1, wherein the therapeutic agent is the gene therapy.

Clause 16. The method of clause 15, wherein the gene therapy is administered by intravenous injection or infusion.

Clause 17. The method of clause 15, wherein the gene therapy is onasemnogene abeparvovec.

Clause 18. The method of clause 17, wherein the onasemnogene abeparvovec comprises vector genomes and at least 0.5×10¹⁴ vector genomes per kilograms of body weight (vg/kg) is administered to the patient.

Clause 19. The method of clause 1, wherein the administration of the therapeutic agent reduces mortality or increases survival in the patient.

Clause 20. A therapeutic agent chosen from one or more of the following:
an antisense oligonucleotide (ASO) targeting SMN1 or SMN2, e.g. targeting intronic splicing silencer N1 (ISS-N1) within SMN2 intron 7;
a mRNA splicing modifier, a pharmaceutically acceptable salt thereof, or a solvate thereof; or
a gene therapy for expressing an SMN1 or SMN2 transcript;
for use in treating a neurodevelopmental disorder, amyotrophic lateral sclerosis, or spinal muscular atrophy in a patient, in an amount effective to treat the neurodevelopmental disorder, amyotrophic lateral sclerosis, or spinal muscular atrophy in a patient, wherein when the patient has a neurodevelopmental disorder, the patient having the neurodevelopmental disorder comprises one or more biallelic variants in GEMIN5 and one or more of a developmental delay, motor dysfunction, cerebellar atrophy, ataxia, and/or hypotonia (e.g., NEDDCAM).

### BRIEF DESCRIPTION OF THE DRAWINGS

FIGS. 1A-1D provide exemplary mRNA (FIG. 1A, SEQ ID NO: 1) and protein (FIG. 1B, SEQ ID NO: 2) sequences for GEMIN 5 and mRNA sequences for SMN1 (FIG. 1C, SEQ ID NO: 3) and SMN2 (FIG. 1D, SEQ ID NO: 4).

FIG. 2 is the chemical structure of nusinersen, an antisense oligonucleotide.

FIGS. 3A and 3B show how GEMIN5 variants reduce the levels of GEMIN5 and SMN assembly proteins. (A) Western blot (WB) analysis of total protein extract from IPSC-derived neuronal cells carrying mono- and bi-allelic L1068P GEMIN5 variants depicting the levels of GEMIN5, GEMIN4, GEMIN6, and GEMIN2, SMN, and U1A, respectively. (B) Quantitative bar plots showing the reduced levels of GEMIN5 and SMN assembly proteins in L1068P homozygous neurons compared to heterozygous controls (two tailed unpaired t test, n=4). (C) Representative WB depicting the expression levels of GEMIN5 and SMN complex proteins in heterozygous and homozygous H913R neurons. (D)-(J) Quantitative comparison of expression levels of (D) GEMIN5, (E) GEMIN4, (F) SMN, (G) GEMIN6, (H) GEMIN2, (I) U1A, and (J) GEMIN3 between H913R^{-/-}and H913R+/−neuronal cells as in panel D (one-way ANOVA-Bonferroni test, n=4). (K) Representative WB showing the proteins levels of SMN, GEMIN4, and GEMIN2 in the patient carrying p.Trp373* and p.Arg1016Cys variants in GEMIN5. Protein lysates were prepared from the PBMCs isolated from the patient and the unaffected parent (Arg1016Cys/+). (L)-(O) Quantitative plot showing the reduced levels of (L) GEMIN5, (M) GEMIN4, (N) SMN, and (O) GEMIN2 proteins in the patient relative to unaffected parent (father). The data represent mean±SEM. P values (***<0.001, **<0.01, *<0.05) are calculated by unpaired t test (n=3). (P) and (Q) Representative WB showing the protein levels of GEMIN5, GEMIN4, and SMN in L1068P (P) heterozygous and (Q) homozygous neurons after 0, 2, 4, 8, 12 and 24 hours of cycloheximide (CHX) treatment. Tubulin was used as normalization control. (R)-(T) Quantitative analysis of the rate of degradation of (R) GEMIN5, (S) SMN and (T) GEMIN4 after CHX treatment showed the increased rate of depletion of GEMIN5 and SMN in homozygous L1068P neurons as compared to heterozygous controls (Nonlinear regression-one phase decay, n=4). (U) and (V) Expression analysis of GEMIN5, SMN, and other GEM-proteins by qPCR in (U) L1068P and (V) H913R neurons. The transcript levels of GEMIN5, GEMIN4, GEMIN3, GEMIN6, GEMIN2, and SMN showed no significant changes among heterozygous and homozygous GEMIN5, L1068P, and H193R variants (two tailed Mann-Whitney U test, n=6). (W) Quantitative PCR showing the relative stability of GEMIN5 mRNA between hetero and homozygous L1068P neurons by using total RNA isolated at 0, 1, 2, 4, 6, and 8 hours after actinomycin D treatment (Nonlinear regression-one phase decay, n=4). The data represent mean±SEM. P values (****<0.0001, ***<0.001, **<0.01).

FIGS. 4A-4C depict how the loss of GEMIN5 over 50% leads to decreased levels of snRNP complex proteins in HEK293T cells. (FIG. 4E) Representative WB showing the effect of the best GEMIN5 shRNAs on the levels of GEMIN4, GEMIN3, GEMIN2, GEMIN6, SMN, SmB1/B2, and U1A as compared to scrambled control. GEMIN5 shRNA B was used in combination with GEMIN5 shRNA 5 and 4 to obtain the maximum knockdown efficiency. a tubulin was used as internal control. Quantitative analysis from displaying the relative expression levels of (FIG. 4F) GEMIN5, and (FIG. 4J) SMN (one-way ANOVA-Bonferroni test, n=5). The data represent mean±SEM. P values (****<0.0001, ***<0.001, **<0.01).

FIG. 5 shows the effect of increased levels of GEMIN5 on expression of SMN complex proteins. (A) Representative immunoblots showing the levels of GEMIN5, SMN, U1A, SmB1/B2, and other GEM proteins after ectopic overexpression of GEMIN5 plasmid construct in HEK293T cells. (B)-(I). Quantitative bar graphs showing significant and increase in (B) GEMIN5 and (C) GEMIN4 after dosage-dependent overexpression of GEMIN5 and as shown in panel A. No significant change was observed in the levels of (D) SMN, (E) GEMIN3, (F) GEMIN6, (G) GEMIN2, (H) U1A, and (I) SnB1/B2 levels by GEMIN5 overexpression. P values (***<0.001, **<0.01, n.s) are of One-way analysis of variance (ANOVA) and post-hoc Bonferroni test.

FIG. 6 shows how biallelic variants in GEMIN5 disrupts SMN assembly formation in vitro. (A) Representative gel image showing the in vitro snRNP assembly formation by using 3′ Cy3-biotin labeled Ul snRNA and the cytoplasmic extract from hetero- and homozygous L1068P and H913R neurons as well as from the HEK293T cells transfected with GEMIN5 shRNA. (B) Quantitative analysis of the in-vitro SMN complex formation shown in panel A (one-way ANOVA-Bonferroni test, n=3). (C) Immunoprecipitation blot showing the reduced interaction of HA tagged Leu1068Pro and His913Arg variants with GEMIN4, GEMIN3, and SMN as compared to HA-GEMIN5 WT protein in HEK cells. (D) Diagrammatic representation showing the possible mode of disruption in snRNP complex formation due to loss of GEMIN5 in L1068P and H913R variants. The data represent mean±SEM P-values *<0.01, **<0.05.

FIG. 7 shows how the loss of GEMIN5 leads to developmental defects and motor dysfunction in Drosophila. (A) Flow diagram comparing different developmental stages of flies between rigor mortis RNAi and W1118 control flies. (B) and (C) RNAi-mediated knockdown of rigor mortis, as determined by qPCR in (A), resulted in (B) pupal lethality and (C) eclosion defects as measured by percentage of eclosed adult homozygous flies (two tailed Unpaired t test, n=5). The RNAi transgene under inducible tubulin-UAS gal4 system was expressed by growing the larvae on 1 mM RU486 drug food. (D) Bar graph representing rapid iterative negative geotaxis (RING) assay, calculated as climbing speed of a fly per second, showed significant defects in the climbing velocities of flies with RNAi-mediated GEMIN5 KD as compared to controls. The effect was apparent when the transgene was expressed for 20 day on 20 mM RU486 drug food under the control of tubulin-GS driver (two tailed Mann-Whitney U test, n=25-39). (E) Kaplan-Meier survival plot showing the effect of the loss of endogenous rig mortis on the life span of flies. The flies were grown on 20 mM RU486 drug food to express the rig mortis RNAi transgene and monitored every day for the span of 45 days (Log-rank (Mantel-Cox) test, n=80). The data represent mean±SEM. P values (****<0.0001, ***<0.001, **<0.01).

FIGS. 8A-8C show SMN upregulation and its result on Gemin5 neurodegeneration in vivo. (A) Schematic representing the experimental to investigating the genetic manipulation of the Drosophila snRNP complex proteins in the background of loss of Gemin5 (Rig) animals in vivo. Drosophila expressing UAS-Rig RNAi were crossed with the GMR-gal4 driver for targeted expression to the Drosophila eye and Tubulin-gal4 driver for ubiquitous expression. (B) Representative images of female Drosophila eyes expressing GMR-gal4 and Rig RNAi crossed with control (W1118), Luciferase OE, Smn RNAi, and Smn OE. (C), (D), and (E) Quantification of eye degeneration of control and Rig RNAi flies combined with (C) Luciferase OE, (D) Smn RNAi, and (E) Smn OE (n=20 Drosophila per group, One-way ANOVA w/Tukey's multiple comparisons). Error bars indicate S.E.M.**** p<0.0001. NS, not significant. (F) Quantification of relative eye sizes of control and Rig RNAi flies combined with Smn OE (n=5 Drosophila per group, One-way ANOVA w/Tukey's multiple comparisons). Error bars indicate S.E.M. * p<0.05; **** p<0.001. NS, not significant. (G) and (H) qPCR analysis from (n=4 biological replicates of 6 Drosophila heads per group) confirms significant knockdown of endogenous (G) Rig in the Rig KD groups and significant overexpression of (H) Smn in the Smn OE crosses (One-way ANOVA w/Tukey's multiple comparisons). Error bars indicate S.E.M. * p<0.05; **** p<0.0001; NS, not significant. (I) Representative male images of eclosed Drosophila adults expressing Control, Rig RNAi (pupal lethality), Rig RNAi in combination with over expression of Smn, and Smn OE. (J) The percentage of eclosed adults for control and Rig RNAi animals combined with Smn OE (n=100 Drosophila per replicate, One-way ANOVA w/Tukey's multiple comparisons). Error bars indicate S.E.M. **** p<0.0001.

FIG. 9 shows how the effect of SMN expression on the levels of GEMIN5 in HEK293T cells. (A) WB analysis showing the levels of GEMIN5 and SMN proteins after ectopic overexpression of EGFP-SMN at 1 μg and 2 μg in HEK293T cells (EGFP alone and untransfected HEK293T cells were used as control). (B) Quantification of proteins from panel A showing a significant increase in GEMIN5 and SMN after over expression of SMN (n=3 blots per condition, unpaired students t test between Oug and each condition). Error bars indicate S.E.M. * p<0.05; ** p<0.01; *** p<0.001; **** p<0.0001. NS, not significant. (C) WB analysis showing the levels of GEMIN5 and SMN after dose-dependent administration of the SMN2 oligonucleotide ASO N1. (D) and (E) Quantification of GEMIN5 and SMN protein levels after administration of 0, 25, 50, & 100 nm of (D) control ASO and (E) ASO N1 (n=3 blots per condition, unpaired students t-test between Onm and each condition). Error bars indicate S.E.M. * p<0.05; ** p<0.01; *** p<0.001; **** p<0.0001. NS, not significant. (F) and (G) Quantification of the mRNA levels showing a significant increase in (F) GEMIN5 and (G) SMN after EGFP-SMN expression (n=3, One-way ANOVA w/Bonferroni test). Error bars indicate S.E.M. *** p<0.001; **** p<0.0001. (H) and (I) Quantification of GEMIN5 and SMN2-FL mRNA levels after administration of 0, 25, 50, & 100 nm of (H) control ASO and (I) ASO N1 (n=4, unpaired students t-test between Onm and each condition). Error bars indicate S.E.M. * p<0.05; ** p<0.01; *** p<0.001; **** p<0.0001. NS, not significant.

FIG. 10 shows the result of GEMIN5 in motor neurons derived from SMA patient iPSC lines. (A) WB analysis showing the levels of GEMIN5 and SMN in motor neurons differentiated from iPSCs from two healthy controls and 6 SMA patients (type 1, type 2 & type 3). (B) and (C) Quantification of (B) GEMIN5 and (C) SMN showing a significant decrease in both proteins in SMA type 1 motor neurons compared to two healthy controls (n=3 biological replicates from 3 independent differentiations, unpaired students t-test). Error bars indicate S.E.M. ** p<0.01; *** p<0.001. (D) and (E). Quantification of (D) GEMIN5 and (E) SMN showing a decrease in both proteins in SMA type 2 motor neurons compared to two healthy controls (n=3 biological replicates from 3 independent differentiations, unpaired students t-test). Error bars indicate S.E.M. * p<0.05. NS, not significant. (F) and (G) Quantification of (F) GEMIN5 and (G) SMN showing a significant decrease in only SMN protein in SMA type 3 motor neurons compared to two healthy controls (n=3 biological replicates from 3 independent differentiations, unpaired students t-test). Error bars indicate S.E.M. **** p<0.0001. NS, not significant. (H) Pearson correlation of SMN and GEMIN5 levels in the healthy controls and SMA type 1 motor neurons exhibits a strong positive correlation between the levels of both proteins across all samples (R=0.93) (n=average of 3 biological replications, Pearson correlation coefficient). (I) Pearson correlation of SMN and GEMIN5 levels in the healthy controls and 2 SMA type 2 motor neurons exhibits a strong positive correlation between the levels of both proteins across all samples (R=0.95) (n=average of 3 biological replicates, Pearson correlation coefficient). (J) Pearson correlation of SMN and GEMIN5 levels in the healthy controls and 3 SMA type 3 motor neurons exhibits a moderate positive correlation between the levels of both proteins across all samples (R=0.5) (n=average of 3 biological replicates, Pearson correlation coefficient).

FIG. 11 shows how the upregulation of SMN impacts the levels of GEMIN5 in mutant GEMIN5 iPSC derived neurons. (A) Representative blots showing the levels of GEMIN5 and SMN after lentiviral transduction of EGFP and EGFP-SMN in unaffected control (H913R/+) and patient (H913R/H913R) GEMIN5^{H913R} iPSC neurons. (B) and (C). Quantification of (B) GEMIN5 and (C) SMN proteins from GEMIN5^{H913R} iPSC neurons in (A). (n=4 blots per condition, One-way ANOVA w/Tukey's multiple comparisons). Error bars indicate S.E.M. * p<0.05; ** p<0.01; **** p<0.0001; NS, not significant. (D) Representative blots showing the levels of GEMIN5 and SMN after lentiviral transduction of EGFP and EGFP-SMN in mutant GEMIN5^{L1068P} iPSC derived neurons. (E) and (F) Quantification of (E) GEMIN5 and (F) SMN proteins from mutant derived GEMIN5^{L1068P} iPSC neurons in panel D. (n=8 blots per condition, unpaired students t-test). Error bars indicate S.E.M. ** p<0.01; **** p<0.0001. (G, intentionally omitted) Representative confocal images of mutant GEMIN5^{L1068P} derived iPSC neurons transduced with EGFP control and lentiviral EGFP-SMN from the same set of neuronal differentiations. The neurons were probed for EGFP, endogenous SMN (to assess the level of SMN OE), and endogenous GEMIN5. The cell nuclei were stained with DAPI. Scale bar=10 μm and 5 μm for zoom. (H) and (I) Quantitative analysis displaying the total intensity of (H) SMN and (I) GEMIN5 from panel G measured as integrated density values (IDV) divided by cell area (n=170 neurons, unpaired students t-test). Error bars indicate S.E.M. **** p<0.0001. (J) Representative blots showing the protein levels of GEMIN5 and SMN in mutant GEMIN5^{L1068P} neurons after administration of ASO Control and ASO N1 at 500 nM. (K) and (L) Quantitative analysis of (K) GEMIN5 and (L) SMN proteins displaying an increase in expression after 500 nM of ASO Control or ASO N1 administration. (n=6 blots, unpaired students t-test). Error bars indicate S.E.M. ** p<0.01; *** p<0.001. (M, intentionally omitted) Representative confocal images of mutant GEMIN5^{L1068P} derived iPSC neurons transfected with 500 nm of Cy3 ASO Control and Cy3 ASO N1 from the same set of neuronal differentiations. The neurons were probed for, endogenous SMN (to assess the level of SMN OE), Cy3, and endogenous GEMIN5. The cell nuclei were stained with DAPI. Scale bar=10 μm and 5 μm for zoom. (N) and (O) Quantitative analysis displaying the total intensity of (N) SMN and (O) GEMIN5 from panel G measured as integrated density values (IDV) divided by cell area (n=60 neurons, unpaired students t-test). Error bars indicate S.E.M. **** p<0.0001. (P) Representative blots showing the protein levels of GEMIN5 and SMN in GEMIN5^{L1068P} neurons after 0, 4, 8, 12 and 24 hours of cycloheximide (CHX) treatment. (Q) and (R) Quantitative analysis of the rate of degradation of (Q) GEMIN5 and (R) SMN proteins after CHX treatment at 0, 4, 8, 12, and 24 hours. (n=3 blots, nonlinear regression-one phase decay).

FIG. 13 shows how SMN and GEMIN5 interact through the RNA-binding sites of GEMIN5 and the Tudor domain of SMN. (A) Schematic illustration of constructed HA-GEMIN5 constructs. HA-GEMIN5-WT harbors full length GEMIN5 protein. HA-WD40 harbors only the n-terminus of GEMIN5 containing 13 tryptophan aspartic acid repeat domains (WD40). HA-ΔRBS lacks the C-terminus of GEMIN5 and harbors the N-terminus and the tetratricopeptide repeat (TPR)-like domains of GEMIN5. HA-ΔWD40 lacks the N-terminus of GEMIN5 and harbors the TPR-like region and the c-terminus containing the RNA-binding sites of GEMIN5. (B) Representative blots of co-immunoprecipitation from HEK cells expressing HA-GEMIN5 constructs and EGFP-SMN. Immunoprecipitation with HA antibody showed SMN in the input samples and pulled down with HA-GEMIN5-WT and HA-ΔWD40, but absent from HA-WD40 and HA-ΔRBS. Immunoprecipitation with SMN showed HA-GEMIN5 in the input samples and pulled down with HA-GEMIN5-WT and AWD40, but absent from HA-WD40 and HA-ΔRBS. (C) Representative blot of Immunoprecipitated HA-GEMIN5-WT and HA-ΔWD40 HEK cell lysates with HA antibody and treated with RNase A. Treatment with RNase A slightly decreases the interaction between SMN and HA-GEMIN5-WT and HA-AWD40. (D) Schematic illustration of constructed HA-SMN constructs. HA-SMN-WT harbors full length SMN protein. HA-SMN Ex1-5 harbors exons 1-5 and lacks exons 6-8 of SMN. HA-SMN AEx3 lacks exon 3 of SMN which harbors the Tudor domain. HA-SMN AEx5 lacks exon 5 which harbors the Proline-Rich region of SMN. (E) Representative blots of immunoprecipitation from HEK cells expressing HA-SMN constructs. Immunoprecipitation with HA antibody showed endogenous GEMIN5 in the input samples and pulled down with HA-SMN-WT and HA-SMN ΔEx5, but absent from HA-SMN Ex1-5 and HA-SMN Ex3. (F) Quantitative analysis of immunoprecipitated endogenous GEMIN5 pulled down with the HA-SMN constructs (n=3 blots, One-way ANOVA w/Tukey's multiple comparisons). Error bars indicate S.E.M. *** p<0.001. NS, not significant. (G) Representative blots of HEK cells transfected with HA-SMN-WT, HA-SMN AEx5, and HA-SMN AEx5 showing the levels endogenous GEMIN5 and SMN. (H) Quantitative analysis shows upregulation of GEMIN5 and SMN protein in HA-SMN-WT and HA-SMN AEx5 only (n=4 blots, One-way ANOVA w/Tukey's multiple comparisons). Error bars indicate S.E.M. ** p<0.01; *** p<0.001.

FIG. 13 shows how the effect of SMN expression on snRNP core assembly in mutant GEMIN5 iPSC neurons. (top) Representative gel displaying complete in vitro snRNP assembly formation using 3′ Cy3-biotin-labeled Ul snRNA and the cytoplasmic extract from control and L1068P neurons with lentiviral SMN and ASO N1 administration. Untransfected HEK293T cells and HEK293T transfected with GEMIN5 shRNA were used as a positive control for assembly formation. (bottom) Representative quantification of 3 biological replicate sample displaying the intensity of the snRNP assembly between groups (n=3, One-way ANOVA w/Tukey's multiple comparisons). Error bars indicate S.E.M. * p<0.05; ** p<0.01. NS, not significant.

FIG. 14: Lentiviral SMN expression significantly impacts the alternative splicing profile in mutant GEMIN5^{H913R} neurons. a. Representative schematic of the five-alternative splicing (AS) event types reported by rMATS. The types of AS events include retained intron (RI), alternative 5′ splice site (A5′SS), alternative 3′ splice site (A3′SS), mutually exclusive exons (MXE), and exon skipping (SE)., b. The number of significant splicing events with an increased or decreased inclusion per AS category in GEMIN5^{H913R} neurons expressing EGFP control and EGFP-SMN compared to control (FDR<0.05, ΔY>5%). GEMIN5^{H913R} neurons expressing EGFP-SMN result in a higher number of increased inclusions and reduce the number of decreased inclusions in GEMIN5^{H913R} neurons expressing EGFP control. c. Percentage of splicing events per category that resulted in an increased inclusion event between mutant GEMIN5^{H913R} neurons with EGFP and SMN OE compared to control. Mutant GEMIN5H913R neurons with SMN saw a rescue in decreased inclusions and an increased percentage in included AS events (SE, A5SS, A3SS, RI) compared to mutant GEMIN5^{H913R} neurons with EGFP. d. Analysis showing the number of genes that exhibited a significantly improved AS event (increased inclusion) per category between mutant GEMIN5^{H913R} neurons with and without lentiviral SMN expression (FDR<0.05, ΔY>5%). e. Gene ontology (GO)-based annotation of the genes with a significantly improved splicing event after SMN transduction via DAVID (v6.7). The y axis represents GO annotations of the up-key words. The x axis represents the Fold Enrichment Score for each GO annotation (FDR<0.05).

### DESCRIPTION OF THE INVENTION

Other than in the operating examples, or where otherwise indicated, the use of numerical values in the various ranges specified in this application are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values. Further, as used herein, all numbers expressing dimensions, physical characteristics, processing parameters, quantities of ingredients, reaction conditions, and the like, used in the specification and claims are to be understood as being modified in all instances by the term “about”. Moreover, unless otherwise specified, all ranges disclosed herein are to be understood to encompass the beginning and ending range values and any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more and ending with a maximum value of 10 or less, e.g., 1 to 3.3, 4.7 to 7.5, 5.5 to 10, and the like.

As used herein “a” and “an” refer to one or more. The term “comprising” is open-ended and may be synonymous with “including”, “containing”, or “characterized by”. The term “consisting essentially of” limits the scope of a claim to the specified materials or steps and those that do not materially affect the basic and novel characteristic(s) of the claimed invention.

As used herein, spatial or directional terms, such as “left”, “right”, “inner”, “outer”, “above”, “below”, “over”, “under”, and the like, relate to the invention as it is shown in the drawing figures are provided solely for ease of description and illustration, and do not imply directionality, unless specifically required for operation of the described aspect of the invention. It is to be understood that the invention can assume various alternative orientations and, accordingly, such terms are not to be considered as limiting.

Unless stated otherwise, nucleotide sequences are recited herein in a 5′ to 3′ direction, and amino acid sequences are recited herein in an N-terminal to C-terminal direction according to convention.

The terms “transfect”, “transfection”, “transfected”, and like terms refer to the introduction of a gene into a eukaryotic cell, such as a keratinocyte, and includes “transduction,” which is viral-mediated gene transfer, for example, by use of recombinant AAV, adenovirus (Ad), retrovirus (e.g., lentivirus), or any other applicable viral-mediated gene transfer platform.

By “expression” or “gene expression,” it is meant the overall flow of information from a gene. A “gene” is a functional genetic unit for producing a gene product, such as RNA or a protein in a cell, or other expression system encoded on a nucleic acid and generally comprising: a transcriptional control sequence, such as a promoter and other cis-acting elements, such as transcriptional response elements (TREs) and/or enhancers; an expressed sequence that typically encodes a protein (referred to as an open-reading frame or ORF) or functional/structural RNA; and a polyadenylation sequence). A gene produces a gene product (typically a protein, optionally post-translationally modified or a functional/structural RNA) when transcribed. By “expression of genes under transcriptional control of,” or alternately “subject to control by” a designated sequence such as a promotor, it is meant gene expression from a gene containing the designated sequence operably linked (functionally attached, typically in cis) to the gene. A gene that is “under transcriptional control” of a promotor or transcription control element, is a gene that is transcribed at detectably different levels in the presence of a transcription factor, e.g., in specific cells, as further described below, and in the context of the present disclosure, produces a difference in transcription levels when expressed in a specific cell type. A “gene for expression of” a stated gene product is a gene capable of expressing that stated gene product when placed in a suitable environment, that is, for example, when transformed, transfected, transduced, etc. into a cell, and subjected to suitable conditions for expression. In the case of a constitutive promoter “suitable conditions” means that the gene typically need only be introduced into a host cell. In the case of an inducible promoter, “suitable conditions” means when factors that regulate transcription, such as DNA-binding proteins, are present or absent, for example, an amount of the respective inducer is available to the expression system (e.g., cell), or factors causing suppression of a gene are unavailable or displaced-effective to cause expression of the gene.

Production of useful nucleic acid constructs, such as recombinant viral vectors for production of nucleic acids, such as the genetic constructs and recombinant viral genomes described herein, is routine, in that molecular cloning and gene assembly methods are routine. Further, a number of companies can custom-synthesize and verify multi-kilobase genes, making the production of genes or genomes as described herein, such as rAAV or scAAV genomes, routine (See, e.g., Gene Synthesis Handbook, 2d Edition, 2014, GenScript USA, Inc.).

AAV (adeno-associated virus), is a virus belonging to the genus Dependoparvovirus, and family Parvoviridae. The virus is a small replication-defective, non-enveloped virus. AAV is not currently known to cause any disease by itself. AAV requires a helper virus, such as adenovirus or herpes simplex virus, to facilitate productive infection and replication. In the absence of helper virus, AAVs establish a latent infection within the cell, either by site-specific integration into the host genome or by persisting in episomal forms. Gene therapy vectors using AAV can infect both dividing and quiescent cells. Furthermore, AAV serotypes have different tropism and can infect cells of multiple diverse tissue types. While eleven serotypes of AAV have been identified to date, AAV2 was among the first to be identified and has been consistently used for the generation of recombinant AAV vectors. Further certain natural or modified AAVs transduce specific organs or cell populations. In one example, AAV-PHP.eB and AAV-PHP.S, capsids efficiently transduce the central and peripheral nervous systems, respectively, when administered intravenously (Chan, K. Y., et al. Engineered AAVs for efficient noninvasive gene delivery to the central and peripheral nervous systems (2017) Nat. Neurosci 20 (8): 1172-1179). For example, compared to AAV9, AAV-PHP.B delivers genes to the brain and spinal cord at least 40 times more efficiently. See also, Tervo, D G, et al. A Designer AAV Variant Permits Efficient Retrograde Access to Projection Neurons (2016) Neuron 92, 372-382, describing engineered AAV variants, e.g., rAAV2-retro, which permit robust retrograde access to projection neurons with efficiency comparable to classical synthetic retrograde tracers, and enable sufficient sensor/effector expression for functional circuit interrogation and in vivo genome editing in targeted neuronal populations.

The AAV virion shell is approximately 25 nanometers (nm) in diameter and encapsulates a single-stranded DNA genome that consists of two large open reading frames (ORFs) flanked by inverted terminal repeats (ITR). The ITRs are the only cis-acting elements required for genome replication and packaging. In wild-type AAV, the left ORF encodes four replication proteins responsible for site-specific integration, nicking, and helicase activity, as well as regulation of promoters within the AAV genome. AAV possesses a 4.7 kb genome, and as such, efficient packaging of recombinant AAV (rAAV) vectors can be performed with constructs ranging from 4.1 kb to 4.9 kb in size (see, e.g., Samulski, R J, et al., AAV-Mediated Gene Therapy for Research and Therapeutic Purposes, Annu. Rev. Virol. 2014. 1:427-51).

Helper-free production of the rAAV requires transfection of the following components into host cells, typically 293 cells (HEK293 cells), which are broadly available, or similar cell lines: (1) an rAAV vector containing the transgene expression cassette flanked by the two ITRs; (2) expression of Rep and Cap proteins, typically provided by a helper plasmid in trans; and (3) adenovirus genes encoding E1, E2A, E4, and virus-associated RNA, also provided, at least in part by another helper plasmid in trans (293 cells produce the Ad E1 gene in trans). Rep and Cap proteins, which are necessary for viral packaging, are replication proteins and capsid proteins, respectively. Rep proteins consist of rep 78, 68, 52 and 40. They specifically are involved with the replication of AAV. Cap proteins are comprised of three proteins, VP1, VP2 and VP3, with molecular weight of 87, 72 and 62 kDa, respectively. These capsid proteins assemble into a near-spherical protein shell of 60 subunits. Helper-free AAV packaging systems are broadly available, for example, from Clontech of Mountain View, California, from Cell Biolabs, Inc. of San Diego, CA, and see, e.g., U.S. Pat. Nos. 6,093,570, 6,458,587, 6,951,758, and 7,439,065. In scAAV (self-complementary AAV), the right ITR contains a deletion of D-sequence (the packaging signal) and a terminal resolution site mutation (Δtrs), which prevent Rep-mediated nicking and force packaging of dimer or self-complementary genomes. Making dsAAV from scAAV vector renders much improved transduction both in vitro and in vivo (see, e.g., pscAAV-MCS Expression vector, Product Data Sheet, Cell Biolabs, Inc., San Diego, California (2012-2016)).

Preparation of rAAV transducing particles, such as scAAV transducing particles is routine. Since the transfection method is often considered unsuitable for large-scale production, the infection of cell lines stably expressing Rep and Cap with adenovirus carrying a vector genome has afforded the ability to scale-up. Another option includes infection of proviral cell lines with adenovirus or herpes simplex virus vector carrying an AAV Rep and Cap expression cassette. These methods still require the complete elimination of adenovirus (or herpesvirus) during the production process. However, in baculovirus expression vector systems for rAAV vector production in insect SF9 cells, the components of AAV production, including Rep and Cap proteins, as well as vector genomes are provided by separate recombinant baculoviruses. Ayuso, E., “Manufacturing of recombinant adeno-associated viral vectors: new technologies are welcome”, Molecular Therapy—Methods & Clinical Development (2016) 3, 15049; doi: 10.1038/mtm.2015.49, and Merten, O-W, et al., describe numerous robust current rAAV production methods, though commercial scale-up and validation needs improvement. High viral titers (~10¹²-10¹³ vp/mL) may be required for certain uses described herein. Protocols are available in the literature for concentration and purification of AAV vectors, allowing production of virus at these high concentrations (see, e.g., Gray S J, et al. (2011) Production of recombinant adeno-associated viral vectors and use in in vitro and in vivo administration. Curr Protoc Neurosci. doi: 10.1002/0471142301.ns0417s57 and Guo P, et al. (2012) Rapid and simplified purification of recombinant adeno-associated virus. J Virol Methods 183 (2): 139-146).

Once the virus has been produced in the, e.g., 293 cells, the cells are collected, lysed, and the resultant virus is purified. Density gradient ultracentrifugation, e.g., in cesium chloride or nonionic iodixanol (VISIPAQ™) gradients and column chromatography, such as ion-exchange, heparin-affinity, or mucin-affinity column chromatography, depending on the AAV serotype. Once the rAAV has been purified and concentrated to a suitable concentration, the virus can be used for in vitro cell transduction or for in vivo animal injection at an appropriate MOI (Multiplicity of Infection).

Numerous rAAV vectors have been made containing genes for expressing proteins, and are commercially available. Due to size limitations, genes for use in rAAV vectors typically do not include introns. rAAV vectors also include the 5′ ITR and 3′ ITR flanking the gene, which is referred to as a transgene. Thus a typical rAAV genome has the following structure, in order from 5′ to 3′ on the sense strand: ITR-promoter-transgene ORF-pA-ITR. Methods of molecular cloning of rAAV transgene constructs, preparation of rAAV particles, and storage and use thereof are broadly-known and further technical details are unnecessary for one of ordinary skill in the art to be able to construct useful rAAV vectors, and produce and use rAAV particles as described herein. As indicated above, so long as the gene sequence is less than the packaging limit of rAAV or scAAV, it is useful for production of a transduction particle as described herein.

AAV is but one of many robust and well-characterized viral vectors suited for gene therapy, which also includes, without limitation, gammaretroviruses, lentiviruses, adenovirus, and herpes simplex virus. While AAV is likely preferred in many instances, other safe and effective viral transducing particles can be developed based on the genes described herein for use in the devices, systems and methods described herein.

Likewise, DNA, such as plasmid or other forms of DNA, optionally combined with suitable transfection reagents, such as liposomes or lipid nanoparticles.

A method for treating a neurodevelopmental disorder in a patient is provided. The method comprises administering to the patient a therapeutic agent in an amount effective to treat the neurodevelopmental disorder, wherein the therapeutic agent is chosen from one or more of the following:
an antisense oligonucleotide (ASO) targeting SMN1 or SMN2, e.g. targeting intronic splicing silencer N1 (ISS-N1) with SMN2 intron 7;
a mRNA splicing modifier, a pharmaceutically acceptable salt thereof, or a solvate thereof; or
a gene therapy for expressing an SMN1 or SMN2 transcript.
   The patient having the neurodevelopmental disorder comprises one or more biallelic variants in GEMIN5 and one or more of a developmental delay, cerebellar atrophy, ataxia, and/or hypotonia.

As used herein, a “patient” or “subject” is an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, a horse, and a whale), or a bird (e.g., a duck or a goose).

As used herein, the terms “treating”, or “treatment” refer to a beneficial or desired result, such as improving one of more, or symptoms of a disease. The terms “treating” or “treatment” also include, but are not limited to, alleviation or amelioration of one or more symptoms of the neurodevelopmental disorder described herein. “Treatment” can also mean prolonging survival as compared to expected survival in the absence of treatment.

By “lower” in the context of a disease marker or symptom is meant a clinically-relevant and/or a statistically significant decrease in such level. The decrease can be, for example, at least 10%, at least 20%, at least 30%, at least 40%, or more, down to a level accepted as within the range of normal for an individual without such a disorder. In certain aspects, the decrease is down to a level accepted as within the range of normal for an individual without such disorder which can also be referred to as a normalization of a level. In certain aspects, the reduction is the normalization of the level of a sign or symptom of a disease, a reduction in the difference between the subject level of a sign of the disease and the normal level of the sign for the disease (e.g., to the upper level of normal when the value for the subject must be decreased to reach a normal value, and to the lower level of normal when the value for the subject must be increased to reach a normal level).

As used herein, “biallelic variants” are mutations in both the paternal copy of the gene and the maternal copy of the gene, i.e., in the context of the present invention, mutations in both the paternal copy of the GEMIN5 gene and in the maternal copy of the GEMIN5 gene.

The one or more biallelic variants of the GEMIN5 gene may be p. (Leu1068Pro), p. (Ala1007Thr), p. (His923Pro, p. (His913Arg), p. (Asp704Glu), p. (Gly683Asp), p. (Ser1000Pro) p. (Tyr1282His), p. (Cys1205Trp), p. (Arg1014Gln), p. (Arg1016Cys), p. (Met485Hfs*27), p. (Arg1016Cys), p. (Arg899Pfs*3), p. (Trp373*), p. (Ala994Val), p. (Arg1016Cys), p. (Pro594Arg), p. (Ser543Gly), or combinations thereof. The * indicates the translation termination (stop) codon.

The one or more biallelic variants cause a loss-of-function of GEMIN5 gene. As used herein, “loss-of-function” is a type of mutation in which the altered gene products lacks the molecular function of the wild-type gene. In the context herein, the GEMIN5 variants cause loss-of-function by reducing GEMIN5 protein expression and disrupting snRNP-core complex dynamics.

A patient is diagnosed with the neurodevelopmental disorder described herein by having one or more biallelic variants in the GEMIN5 gene and one or more of the following: a developmental delay, cerebellar atrophy as determined by an imaging technique, e.g. magnetic resonance imaging (MRI), ataxia, and/or hypotonia.

As used herein, a “developmental delay” is a delay in the expected skills of a patient, as compared to another patient of the same age. For example, developmental delay may include, but is not limited to speech delay, motor delay, cognitive delay, social delay, and combinations thereof.

As used herein, “cerebellar atrophy” is the deterioration, e.g. damage or death, of the nerve cells in the cerebellum of the brain.

As used herein, “ataxia” is poor muscle control that causes clumsy, voluntary movements. For example, a patient with ataxia may have difficulty with walking, balance, hand coordination, speech, swallowing, eye movements, and combinations thereof.

As used herein, “hypotonia” is decreased muscle tone.

The clinical process for diagnosing the patient with the neurodevelopmental disorder described herein may including the following steps: (1) evaluating the patient for developmental delay; (2) evaluating the patient for ataxia and/or hypotonia; (3) scanning the brain of the patient using an imaging technique, e.g., MRI, to determine the presence of cerebellar atrophy; and (4) completing genetic testing to determine the presence of one or more biallelic variants in the GEMIN5 gene.

“Therapeutically effective amount” or an “amount effective” as used herein, is intended to include the amount of a therapeutic agent as described herein that, when administered to a subject having a disease, is sufficient to effect treatment of the disease (e.g., by diminishing, ameliorating or maintaining the existing disease or one or more symptoms of disease). The “therapeutically effective amount” may vary depending on the nature of the injury and its causes, how the therapeutic agent is administered, the disease and its severity and the history, age, weight, family history, genetic makeup, the types of preceding or concomitant treatments, if any, and other individual characteristics of the subject to be treated. A “therapeutically-effective amount” also includes an amount of a therapeutic agent that produces some desired local or systemic effect at a reasonable benefit/risk ratio applicable to any treatment.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject being treated. Some examples of materials which can serve as pharmaceutically-acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates, and/or polyanhydrides; (22) bulking agents, such as polypeptides and amino acids (23) serum component, such as serum albumin, HDL and LDL; and (22) other non-toxic compatible substances employed in pharmaceutical formulations.

Pharmaceutically acceptable salts of any of the therapeutic agents described herein also may be used in the methods described herein. Pharmaceutically acceptable salt forms of the therapeutic agents described herein may be prepared by conventional methods known in the pharmaceutical arts, and include as a class veterinarily acceptable salts. For example and without limitation, where a therapeutic agent comprises a carboxylic acid group, a suitable salt thereof may be formed by reacting the compound with an appropriate base to provide the corresponding base addition salt. Non-limiting examples include: alkali metal hydroxides, such as potassium hydroxide, sodium hydroxide and lithium hydroxide; alkaline earth metal hydroxides, such as barium hydroxide and calcium hydroxide; alkali metal alkoxides, such as potassium ethanolate and sodium propanolate; and various organic bases such as piperidine, diethanolamine, and N-methylglutamine.

Non-limiting examples of pharmaceutically-acceptable base salts include: aluminum, ammonium, calcium, copper, ferric, ferrous, lithium, magnesium, manganic, manganous, potassium, sodium, and zinc salts. Salts derived from pharmaceutically acceptable organic non-toxic bases include, without limitation: salts of primary, secondary, and tertiary amines, substituted amines including naturally occurring substituted amines, cyclic amines, and basic ion exchange resins, such as arginine, betaine, caffeine, chloroprocaine, choline, N,N′-dibenzylethylenediamine (benzathine), dicyclohexylamine, diethanolamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine, and tris-(hydroxymethyl)-methylamine (tromethamine).

Non-limiting examples of pharmaceutically-acceptable acid salts include: acetate, adipate, alginate, arginate, aspartate, benzoate, besylate (benzenesulfonate), bisulfate, bisulfite, bromide, butyrate, camphorate, camphorsulfonate, caprylate, chloride, chlorobenzoate, citrate, cyclopentanepropionate, digluconate, dihydrogenphosphate, dinitrobenzoate, dodecylsulfate, ethanesulfonate, fumarate, galacterate, galacturonate, glucoheptanoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, iso-butyrate, lactate, lactobionate, malate, maleate, malonate, mandelate, metaphosphate, methanesulfonate, methylbenzoate, monohydrogenphosphate, 2-naphthalenesulfonate, nicotinate, nitrate, oxalate, oleate, pamoate, pectinate, persulfate, phenylacetate, 3-phenylpropionate, phosphate, phosphonate, and phthalate.

Multiple salts forms are also considered to be pharmaceutically-acceptable salts. Common, non-limiting examples of multiple salt forms include: bitartrate, diacetate, difumarate, dimeglumine, diphosphate, disodium, and trihydrochloride.

As such, “pharmaceutically acceptable salt” as used herein is intended to mean an active ingredient (therapeutic agent) comprising a salt form of any compound as described herein. The salt form preferably confers to the improved and/or desirable pharmacokinetic/pharmodynamic properties of the compounds described herein.

The therapeutic agents described herein can be administered by any effective route. Examples of delivery routes include, without limitation: topical, for example, epicutaneous, inhalational, enema, ocular, otic, and intranasal delivery; enteral, for example, orally, by gastric feeding tube, and rectally; and parenteral, such as, intravenous, intraarterial, intrathecally, intramuscular, intracardiac, subcutaneous, intraosseous, intradermal, intrathecal, intraperitoneal, transdermal, iontophoretic, transmucosal, epidural, and intravitreal, with intracathecal and oral approaches being preferred in many instances. Suitable dosage forms may include single-dose, or multiple-dose vials or other containers, such as medical syringes, containing a composition comprising the therapeutic agent useful for treatment of the neurodevelopmental disorder described herein.

Dosage regimens may be adjusted to provide the optimum desired response (e.g., a therapeutic or prophylactic response). For example, a single bolus may be administered, several divided doses may be administered over time, or the therapeutic agent may be administered continuously or in a pulsed fashion with doses or partial doses being administered at regular intervals, for example, every 10, 15, 20, 30, 45, 60, 90, or 120 minutes, every 2 through 12 hours daily, or every other day, etc., be proportionally reduced or increased as indicated by the exigencies of the therapeutic situation. In some instances, it may be especially advantageous to formulate therapeutic agents in dosage unit form for ease of administration and uniformity of dosage. The specification for the dosage unit forms are dictated by and directly dependent on (a) the unique characteristics of the therapeutic agent and the particular therapeutic or prophylactic effect to be achieved, and (b) the limitations inherent in the art of compounding such a therapeutic agent for the treatment of sensitivity in individuals.

Useful dosage forms include: intrathecal, intravenous, intramuscular, or intraperitoneal solutions, oral tablets, capsules, or liquids, topical ointments or creams, and transdermal devices (e.g., patches). The therapeutic agent may be provided in a sterile solution comprising the therapeutic agent and a solvent, such as water, saline, lactated Ringer's solution, or phosphate-buffered saline (PBS), e.g., as a parenteral dosage form, such as an intrathecal infusion or injection. Additional excipients, such as polyethylene glycol, emulsifiers, salts and buffers may be included in the solution.

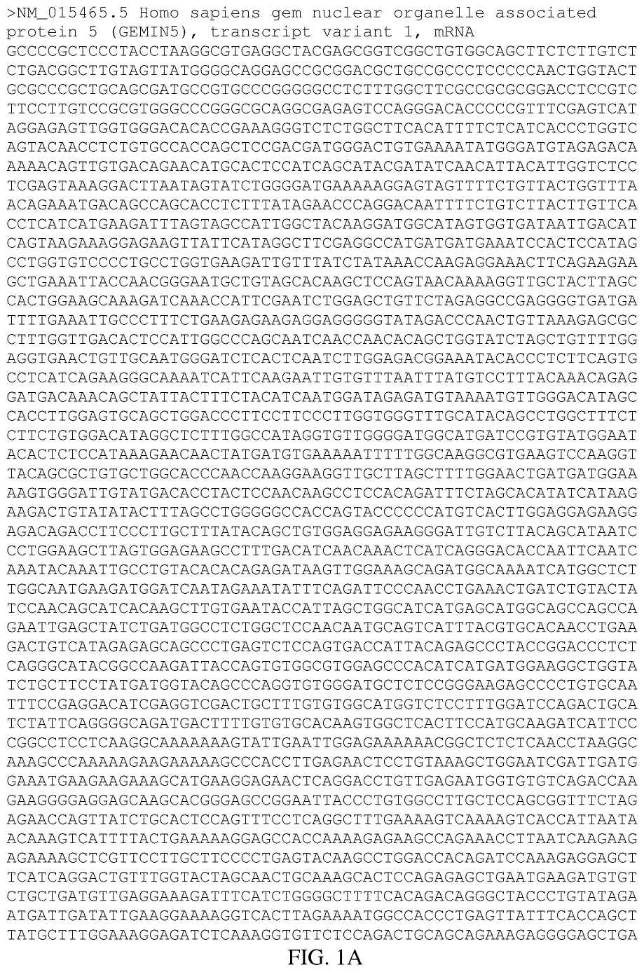

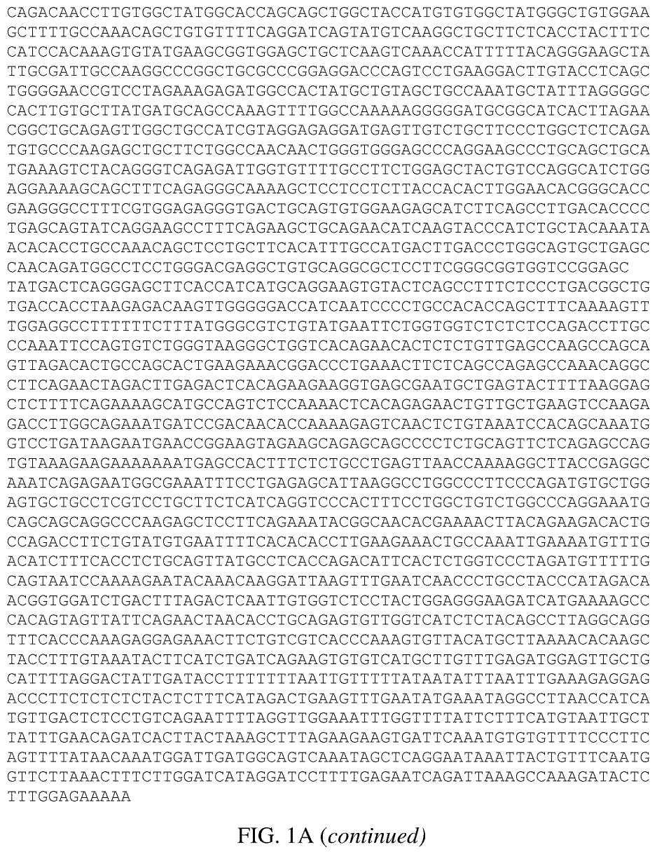

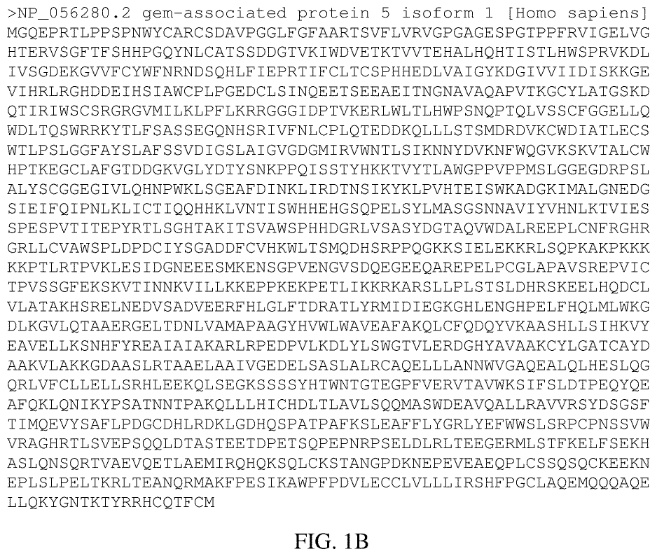

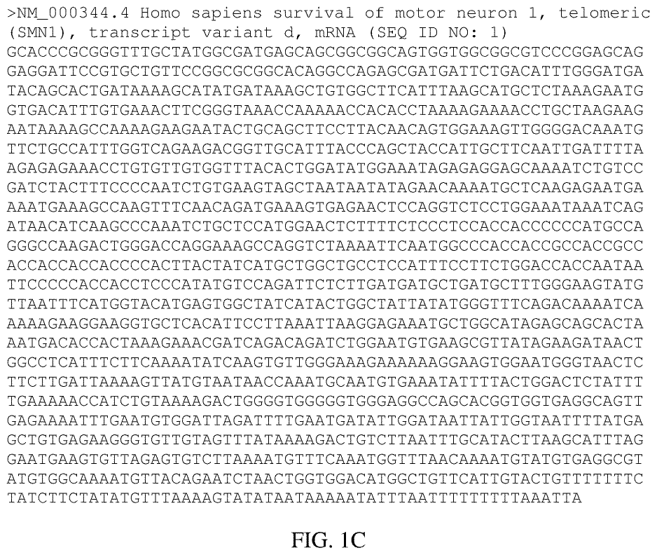

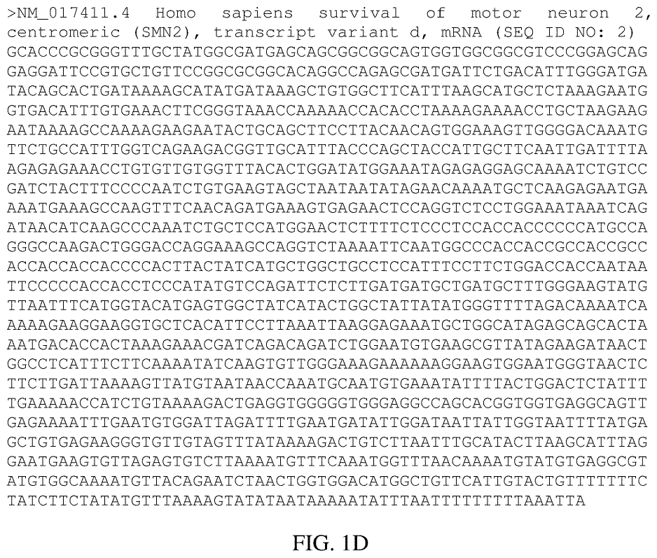

GEMIN5 (e.g., Gene ID: 25929: “GEMIN5 gem nuclear organelle associated protein 5 [Homo sapiens (human)]”, and see, NCBI Reference sequence: NG_052854.1) encodes a WD repeat protein that is a component of the survival of motor neurons (SMN) complex. FIG. 1A provides an exemplary reference sequence for an isoform of GEMIN5 protein sequence (NCBI Reference sequence: NP_056280.2 gem-associated protein 5 isoform 1 [Homo sapiens], corresponding to mRNA sequence provided in NCBI Reference Sequence: NM_015465.5), FIGS. 1A and 1B, SEQ ID NOS: 1 and 2, to which GEMIN5 amino acid references correspond herein.

SMN refers to survival motor neuron protein, exemplary cDNA sequences are provided in FIGS. 1C and 1D. GenBank Ref. No. NM_000344.4 provides an exemplary mRNA sequence (common, longest isoform d) for survival motor neuron 1 (SMN1) (SEQ ID NO: 3). Additional exemplary sequences for SMN1 are broadly-known (See, e.g. Gene ID: 6606). FIG. 1D provides an exemplary mRNA sequence for SMN2 (SEQ ID NO: 4). GenBank Ref. No. NM_017411.4 provides an exemplary mRNA sequence (longest isoform d) for survival motor neuron 2 (SMN2). Additional exemplary sequences for SMN1 are broadly-known (See, e.g. Gene ID: 6607. As described below, antisense oligonucleotides (ASO) and gene therapies, and reagents for producing those agents, such as a lentivirus or AAV vector, may be used in the treatment methods provided herein, and are readily ascertained from mRNA sequences of these genes.

The therapeutic agent may be an antisense oligonucleotide (ASO). As used herein, “antisense oligonucleotides (ASOs)” are single-stranded, chemically-modified chains of multiple nucleotides having a sequence that is complementary to the sequence of the target gene's transcribed messenger RNA (mRNA) within a cell. The ASO may target intronic splicing silencer N1 (ISS-N1) within the SMN2 intron 7. The ASO may alter mRNA splicing of SMN2. For example, the ASO may block the ISS-N1 in the SMN2 gene in order to facilitate the exon 7 inclusion and the generation of full length SMN2 transcripts to increase the level of SMN protein produced. Suitable ASOs that are complementary to intron 7 of the nucleic acid encoding SMN2 pre-mRNA are described in U.S. Pat. No. 9,717,750 B2, U.S. Pat. No. 8,110,560 B2, U.S. Pat. No. 10,266,822, which are incorporated by reference herein in their entireties.

The ASO may be from 12 to 20 nucleotides, e.g. 12, 13, 14, 15, 16, 17, 18, 19, or 20 nucleotides, in length. The nucleotides may be chemically modified to comprise a 2′-O-methoxyethyl (MOE) sugar moiety, a 2′-O-methyl (OMe) sugar moiety, or a morpholino sugar moiety. The nucleotides may be linked through a phosphorothioate linkage.

The ASO may comprise the one of the following sequences as described in Table 1. The nucleotides may be chemically modified to comprise a 2′-methoxyethyl (MOE) sugar moiety, a 2′-O-methyl (OMe) sugar moiety, or a morpholino sugar moiety. The ASO may comprise a sequence having at least 90% sequence identity, at least 95% sequence identity, or 100% sequence identity with one of SEQ ID NOS: 5 to 30.

TABLE 1
ASO Sequences

| Sequence | Length | SEQ ID NO. |
|---|---|---|
| TCACTTTCATAATGCTGG | 18 | 5 |
| TTTCATAATGCTGGC | 15 | 6 |
| TGCTGGCAGACTTAC | 15 | 7 |
| CATAATGCTGGCAGA | 15 | 8 |
| TCATAATGCTGGCAG | 15 | 9 |
| TTCATAATGCTGGCA | 15 | 10 |
| TTTCATAATGCTGGC | 15 | 11 |
| ATTCACTTTCATAATGCTGG | 20 | 12 |
| TCACTTTCATAATGCTGG | 18 | 13 |
| CTTTCATAATGCTGG | 15 | 14 |
| TCATAATGCTGG | 12 | 15 |
| ACTTTCATAATGCTG | 15 | 16 |
| TTCATAATGCTG | 12 | 17 |
| CACTTTCATAATGCT | 15 | 18 |
| TTTCATAATGCT | 12 | 19 |
| TCACTTTCATAATGC | 15 | 20 |
| CTTTCATAATGC | 12 | 21 |
| TTCACTTTCATAATG | 15 | 22 |
| ACTTTCATAATG | 12 | 23 |
| ATTCACTTTCATAAT | 15 | 24 |
| CACTTTCATAAT | 12 | 25 |
| GATTCACTTTCATAA | 15 | 26 |
| TCACTTTCATAA | 12 | 27 |
| TTCACTTTCATA | 12 | 28 |
| ATTCACTTTCAT | 12 | 29 |
| AGTAAGATTCACTTT | 15 | 30 |

The nucleic acid sequences provided herein, including, but not limited to those described in Table 1, are intended to encompass nucleic acids containing any combination of natural or modified RNA and/or DNA, including, but not limited to such nucleic acids having modified nucleobases. By way of example and without limitation, an ASO having a nucleobase sequence “ATCGATCG” encompasses any oligomeric compounds having such a nucleobase sequence, whether modified or unmodified, including, but not limited to such compounds comprising RNA bases, such as those having sequence “AUCGAUCG” and those having some DNA bases and some RNA bases such as “AUCGATCG” and oligomeric compounds having other modified bases such as “ATMeCGAUCG” wherein MeC indicates a cytosine base comprising a methyl group at the 5-position.

For example, the ASO therapeutic agent may be nusinersen, which is a 2′-O-(2-methoxyethyl) modified phosphorothioate linked ASO or a pharmaceutically acceptable salt or solvate thereof, comprising the following sequence:

(SEQ ID NO. 31)
5′-^{Me}U^{Me}CA^{MeMe}u^{Me}u^{Me}u^{Me}CA^{Me}UAA^{Me}UG^{Me}C^{Me}UGG-3′

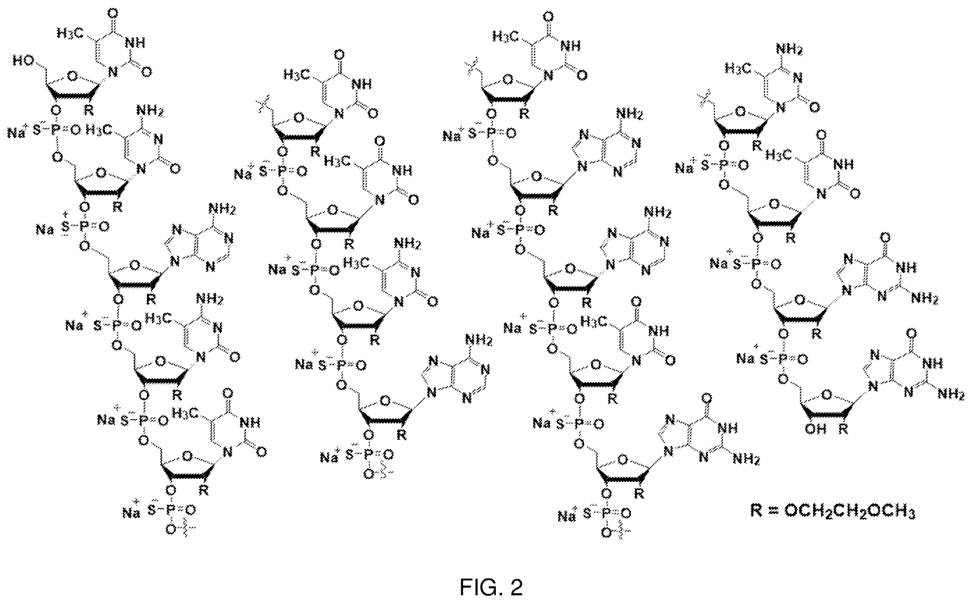

The structure of nusinersen is provided in FIG. 2. Nusinersen is commercially available as SPINRAZA® (Biogen, Cambridge, MA).

The ASO, the pharmaceutically-acceptable salt of the ASO, or the solvate of the ASO may be in the form of an intrathecal injectable solution. Injectable solutions include an effective amount of ASO of at least about 0.5 milligrams per milliliter (mg/mL), at least 1 mg/mL, at least 2 mg/mL, at least 3 mg/mL, from at least 0.5 mg/mL to at least 3 mg/mL, from at least 1 mg/mL to at least 3 mg/mL, from at least 2 mg/mL to at least 3 mg/mL, such as 2.4 mg/mL.

The ASO, the pharmaceutically-acceptable salt of the ASO, or the solvate of the ASO may be delivered to the patient intrathecally in a dosing regimen. For example, the ASO, the pharmaceutically-acceptable salt of the ASO, or the solvate of the ASO may be administered to the patient in four loading doses and then may be administered every four months thereafter. For example, in the first year, the ASO, the pharmaceutically-acceptable salt of the ASO, or the solvate of the ASO may be administered intrathecally, followed by a second intrathecal injection approximately 14 days later, followed by a third intrathecal injection approximately 14 days after the second intrathecal injection, and followed by a fourth intrathecal injection approximately 30 days after the third intrathecal injection. The patient may then be administered a fifth intrathecal injection approximately 4 months after the fourth intrathecal injection, a sixth intrathecal injection approximately 4 months after the fifth intrathecal injection, and a seventh intrathecal injection approximately 4 months after the sixth intrathecal injection. The patient may then be administered intrathecal injections every approximately 4 months thereof.

The therapeutic agent may be a small molecule mRNA splicing modifier. The mRNA splicing modifier may modify the splicing of SMN2 mRNA to include exon 7. For example, the mRNA splicing modifier may comprise the following general structure of Formula I, as described in U.S. Pat. No. 9,586,955 B2, which is incorporated herein by reference in its entirety:

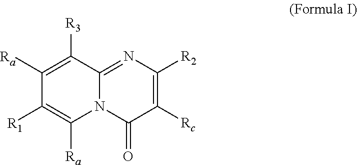

or a free acid, free base, a chloride salt, hydrobromide salt, hydrochloride salt, dihydrochloride salt, acetate salt, trifluoroacetate salt, trifluoroacetic acid salt, isotopologue, stereoisomer, racemate, enantiomer, diastereomer or tautomer thereof, wherein Riis a heterocyclyl, i.e. azetidinyl, tetrahydrofuranyl, pyrrolidinyl, piperidinyl, piperazinyl, 1,4-diazepanyl, 1,2,5,6-tetrahydropyridinyl, 1,2,3,6-tetrahydropyridinyl, hexahydropyrrolo[3,4-b]pyrrol-(1H)-yl, (3aS,6aS)-hexahydropyrrolo[3,4-b]pyrrol-(1H)-yl, (3aR,6aR)-hexahydropyrrolo[3,4-b]pyrrol-(1H)-yl, hexahydropyrrolo[3,4-b]pyrrol-(2H)-yl, (3aS,6aS)-hexahydropyrrolo[3,4-b]pyrrol-(2H)-yl, hexahydropyrrolo[3,4-c]pyrrol-(1H)-yl, (3aR,6aS)-hexahydropyrrolo[3,4-c]pyrrol-(1H)-yl, octahydro-5H-pyrrolo[3,2-c]pyridinyl, octahydro-6H-pyrrolo[3,4-b]pyridinyl, (4aR,7aR)-octahydro-6H-pyrrolo[3,4-b]pyridinyl, (4aS,7aS)-octahydro-6H-pyrrolo[3,4-b]pyridinyl, hexahydropyrrolo[1,2-a]pyrazin-(2H)-one, hexahydropyrrolo[1,2-a]pyrazin-(1H)-yl, (7R,8aS)-hexahydropyrrolo[1,2-a]pyrazin-(1H)-yl, (8aS)-hexahydropyrrolo[1,2-a]pyrazin-(1H)-yl, (8aR)-hexahydropyrrolo[1,2-a]pyrazin-(1H)-yl, (8aS)-(8aR)-octahydropyrrolo[1,2-a]pyrazin-(1H)-yl, octahydropyrrolo[1,2-a]pyrazin-(1H)-yl, octahydro-2H-pyrido[1,2-a]pyrazinyl, 3-azabicyclo[3.1.0]hexyl, (1R,5S)-3-azabicyclo[3.1.0]hexyl, 8-azabicyclo[3.2.1]octyl, (1R,5S)-8-azabicyclo[3.2.1]octyl, 8-azabicyclo[3.2.1]oct-2-enyl, (1R,5S)-8-azabicyclo[3.2.1]oct-2-enyl, 9-azabicyclo[3.3.1]nonyl, (1R,5S)-9-azabicyclo[3.3.1]nonyl, 2,5-diazabicyclo[2.2.1]heptyl, (1S,4S)-2,5-diazabicyclo[2.2.1]heptyl, 2,5-diazabicyclo[2.2.2]octyl, 3,8-diazabicyclo[3.2.1]octyl, (1R,5S)-3,8-diazabicyclo[3.2.1]octyl, 1,4-diazabicyclo[3.2.2]nonyl, azaspiro[3.3]heptyl, 2,6-diazaspiro[3.3]heptyl, 2,7-diazaspiro[3.5]nonyl, 5,8-diazaspiro[3.5]nonyl, 2,7-diazaspiro[4.4]nonyl and 6,9-diazaspiro[4.5]decyl, wherein, the heterocyclyl is optionally substituted with one, two or three R₃ substituents and optionally, with one additional R₄ substituent; or, wherein, heterocyclyl is optionally substituted with one, two, three or four R₃ substituents; R₂ is heteroaryl; wherein, heteroaryl is optionally substituted with one, two or three R₆ substituents and optionally, with one additional R₇ substituent; Rₐ is, in each instance, independently selected from hydrogen, halogen or C₁₋₈alkyl; R_{b} is hydrogen, halogen, C₁₋₈alkyl or C₁₋₈alkoxy; Rcis hydrogen, halogen or C₁₋₈alkyl; R₃ is, in each instance, independently selected from cyano, halogen, hydroxy, oxo, C₁₋₈alkyl, e.g. methyl, ethyl, propyl, isopropyl and tert-butyl, halo-C₁₋₈alkyl, C₁₋₈alkyl-carbonyl, C₁₋₈alkoxy, halo-C₁₋₈alkoxy, C₁₋₈alkoxy-C₁₋₈ alkyl, C₁₋₈alkoxy-carbonyl, amino, C₁₋₈alkyl-amino, (C₁₋₈alkyl)₂-amino, amino-C₁₋₈alkyl, C₁-salkyl-amino-C₁₋₈alkyl, (C₁₋₈alkyl)₂-amino-C₁₋₈alkyl, amino-C₁₋₈alkyl-amino, C₁₋₈alkyl-amino-C₁₋₈alkyl-amino, (C₁₋₈alkyl-amino-C₁₋₈alkyl)₂-amino, (C₁₋₈alkyl)₂-amino-C₁₋₈alkyl-amino, [(C₁₋₈alkyl)₂-amino-C₁₋₈alkyl]₂-amino, (C₁₋₈alkyl-amino-C₁₋₈alkyl) (C₁₋₈alkyl)amino, [(C₁₋₈ alkyl)₂-amino-C₁₋₈alkyl](C₁₋₈alkyl)amino, C₁₋₈alkoxy-C₁₋₈alkyl-amino, (C₁₋₈alkoxy-C₁₋₈ alkyl)₂-amino, (C₁₋₈alkoxy-C₁₋₈alkyl) (C₁₋₈alkyl)amino, C₁₋₈alkyl-carbonyl-amino, C₁₋₈alkoxy-carbonyl-amino, hydroxy-C₁₋₈alkyl, hydroxy-C₁₋₈alkoxy-C₁₋₈alkyl, hydroxy-C₁₋₈alkyl-amino, (hydroxy-C₁₋₈alkyl)₂-amino or (hydroxy-C₁₋₈alkyl) (C₁₋₈alkyl)amino; R₄ is C₃₋₁₄cycloalkyl, C₃₋₁₄cycloalkyl-C₁₋₈alkyl, C₃₋₁₄cycloalkyl-amino, aryl-C₁₋₈alkyl, aryl-C₁₋₈alkoxy-carbonyl, aryl-sulfonyloxy-C₁₋₈ alkyl, heterocyclyl or heterocyclyl-C₁₋₈alkyl; wherein, each instance of C₃₋₁₄cycloalkyl, aryl and heterocyclyl is optionally substituted with one, two or three R₅ substituents; R₅ is, in each instance, independently selected from halogen, hydroxy, cyano, nitro, C₁₋₈alkyl, halo-C₁₋₈alkyl, C₁₋₈alkoxy, halo-C₁₋₈alkoxy, amino, C₁₋₈alkyl-amino, (C₁₋₈ alkyl)₂-amino or C₁₋₈alkyl-thio; R₆ is, in each instance, independently selected from halogen, hydroxy, cyano, nitro, C₁₋₈alkyl, such as methyl, ethyl, propyl, isopropyl and tert-butyl, C₂-salkenyl, halo-C₁₋₈alkyl, hydroxy-C₁₋₈alkyl, C₁₋₈alkoxy, halo-C₁₋₈alkoxy, C₁₋₈alkoxy-C₁₋₈alkyl, amino, C₁₋₈alkyl-amino, (C₁₋₈alkyl)₂-amino or C₁₋₈alkyl-thio; and, R₇ is C₃₋₁₄cycloalkyl, C₃₋₁₄cycloalkyl-oxy, aryl, heterocyclyl or heteroaryl. In each instance, Rₐ may be hydrogen.

The mRNA splicing modifier comprising the structure of Formula I may be selected from the following: 2-(6-methylimidazo[1,2-a]pyridin-2-yl)-7-(piperazin-1-yl)-4H-pyrido[1,2-a]pyrimidin-4-one, 2-(6-methylimidazo[1,2-a]pyrazin-2-yl)-7-(piperazin-1-yl)-4H-pyrido[1,2-a]pyrimidin-4-one, 2-(6-methylimidazo[1,2-a]pyrazin-2-yl)-7-[(3 S)-3-methylpiperazin-1-yl]-4H-pyrido[1,2-a]pyrimidin-4-one, 2-(6-methoxypyridin-3-yl)-7-(piperazin-1-yl)-4H-pyrido[1,2-a]pyrimidin-4-one, 2-(5-fluoropyridin-3-yl)-7-(piperazin-1-yl)-4H-pyrido[1,2-a]pyrimidin-4-one, 2-(5-fluoropyridin-3-yl)-7-[(3 S)-3-methylpiperazin-1-yl]-4H-pyrido[1,2-a]pyrimidin-4-one, 2-(5-chloropyridin-3-yl)-7-(piperazin-1-yl)-4H-pyrido[1,2-a]pyrimidin-4-one, 2-(5-chloropyridin-3-yl)-7-[(3 S)-3-methylpiperazin-1-yl]-4H-pyrido[1,2-a]pyrimidin-4-one, 2-(5-chloro-6-methoxypyridin-3-yl)-7-(piperazin-1-yl)-4H-pyrido[1,2-a]pyrimidin-4-one, 2-(1H-indol-6-yl)-7-(piperazin-1-yl)-4H-pyrido[1,2-a]pyrimidin-4-one, 2-(1H-indol-5-yl)-7-(piperazin-1-yl)-4H-pyrido[1,2-a]pyrimidin-4-one, 2-(imidazo[1,2-a]pyridin-7-yl)-7-(piperazin-1-yl)-4H-pyrido[1,2-a]pyrimidin-4-one, 2-(imidazo[1,2-a]pyridin-6-yl)-7-(piperazin-1-yl)-4H-pyrido[1,2-a]pyrimidin-4-one, 2-(2-methylimidazo[1,2-a]pyridin-6-yl)-7-(piperazin-1-yl)-4H-pyrido[1,2-a]pyrimidin-4-one, 2-(2-methylimidazo[1,2-a]pyridin-6-yl)-7-(4-methylpiperazin-1-yl)-4H-pyrido[1,2-a]pyrimidin-4-one, 2-(2-methylimidazo[1,2-a]pyridin-6-yl)-7-[(3 S)-3-methylpiperazin-1-yl]-4H-pyrido[1,2-a]pyrimidin-4-one, 7-(1,4-diazepan-1-yl)-2-(2-methylimidazo[1,2-a]pyridin-6-yl)-4H-pyrido[1,2-a]pyrimidin-4-one, 7-[(3R,5 S)-3,5-dimethylpiperazin-1-yl]-2-(2-methylimidazo[1,2-a]pyridin-6-yl)-4H-pyrido[1,2-a]pyrimidin-4-one, 2-(6, 8-dimethylimidazo[1,2-a]pyrazin-2-yl)-7-(piperazin-1-yl)-4H-pyrido[1,2-a]pyrimidin-4-one, 2-(6,8-dimethylimidazo[1,2-a]pyrazin-2-yl)-7-[(3 S)-3-methylpiperazin-1-yl]-4H-pyrido[1,2-a]pyrimidin-4-one, 2-(4, 6-dimethylpyrazolo[1,5-a]pyrazin-2-yl)-7-(piperazin-1-yl)-4H-pyrido[1,2-a]pyrimidin-4-one, 2-(4, 6-dimethylpyrazolo[1,5-a]pyrazin-2-yl)-7-[(3 S)-3-methylpiperazin-1-yl]-4H-pyrido[1,2-a]pyrimidin-4-one, 7-(piperazin-1-yl)-2-[2-(trifluoromethyl) imidazo[1,2-a]pyridin-6-yl]-4H-pyrido[1,2-a]pyrimidin-4-one, 2-(2-ethylimidazo[1,2-a]pyridin-6-yl)-7-(piperazin-1-yl)-4H-pyrido[1,2-a]pyrimidin-4-one, 2-(2,3-dimethylimidazo[1,2-a]pyridin-6-yl)-7-(piperazin-1-yl)-4H-pyrido[1,2-a]pyrimidin-4-one, 7-(piperazin-1-yl)-2-(1H-pyrrolo[2,3-b]pyridin-5-yl)-4H-pyrido[1,2-a]pyrimidin-4-one, 2-(1-methyl-1H-pyrrolo[2,3-b]pyridin-5-yl)-7-(piperazin-1-yl)-4H-pyrido[1,2-a]pyrimidin-4-one, 7-[(3R,5S)-3,5-dimethylpiperazin-1-yl]-2-(1-methyl-1H-pyrrolo[2,3-b]pyridin-5-yl)-4H-pyrido[1,2-a]pyrimidin-4-one, 7-[(3R,5 S)-3,5-dimethylpiperazin-1-yl]-2-(4,6-dimethylpyrazolo[1,5-a]pyrazin-2-yl)-4H-pyrido[1,2-a]pyrimidin-4-one, 7-(1,4-diazepan-1-yl)-2-(4,6-dimethylpyrazolo[1,5-a]pyrazin-2-yl)-4H-pyrido[1,2-a]pyrimidin-4-one, 2-(2-methyl-1,3-benzoxazol-6-yl)-7-(piperazin-1-yl)-4H-pyrido[1,2-a]pyrimidin-4-one, 2-(2-methyl-1,3-benzoxazol-6-yl)-7-[(3S)-3-methylpiperazin-1-yl]-4H-pyrido[1,2-a]pyrimidin-4-one, 2-(2-methyl-1,3-benzothiazol-5-yl)-7-[(3 S)-3-methylpiperazin-1-yl]-4H-pyrido[1,2-a]pyrimidin-4-one, 2-(2-methyl-1,3-benzothiazol-5-yl)-7-(piperazin-1-yl)-4H-pyrido[1,2-a]pyrimidin-4-one, 2-(2-methyl-2H-indazol-5-yl)-7-(piperazin-1-yl)-4H-pyrido[1,2-a]pyrimidin-4-one, 2-(2-methyl-2H-indazol-5-yl)-7-[(3S)-3-methylpiperazin-1-yl]-4H-pyrido[1,2-a]pyrimidin-4-one, 2-(2,8-dimethylimidazo[1,2-a]pyridin-6-yl)-7-(piperazin-1-yl)-4H-pyrido[1,2-a]pyrimidin-4-one, 2-(2-methyl-1,3-benzoxazol-5-yl)-7-(piperazin-1-yl)-4H-pyrido[1,2-a]pyrimidin-4-one, 2-(2-methyl-1,3-benzoxazol-5-yl)-7-[(3S)-3-methylpiperazin-1-yl]-4H-pyrido[1,2-a]pyrimidin-4-one, 7-(1,4-diazepan-1-yl)-2-(2-methyl-1,3-benzothiazol-5-yl)-4H-pyrido[1,2-a]pyrimidin-4-one, 7-[(3 S)-3-methylpiperazin-1-yl]-2-(4-methyl-1,3-thiazol-2-yl)-4H-pyrido[1,2-a]pyrimidin-4-one, 2-(4-methyl-1,3-thiazol-2-yl)-7-(piperazin-1-yl)-4H-pyrido[1,2-a]pyrimidin-4-one, 7-(4-methyl-1,4-diazepan-1-yl)-2-(2-methylimidazo[1,2-a]pyridin-6-yl)-4H-pyrido[1,2-a]pyrimidin-4-one, 2-(4,6-dimethylpyrazolo[1,5-a]pyrazin-2-yl)-7-(4-methylpiperazin-1-yl)-4H-pyrido[1,2-a]pyrimidin-4-one, 2-(2-methyl-1,3-benzoxazol-6-yl)-7-(4-methylpiperazin-1-yl)-4H-pyrido[1,2-a]pyrimidin-4-one,7-[(3R,5S)-3,5-dimethylpiperazin-1-yl]-2-(2-methyl-1,3-benzoxazol-6-yl)-4H-pyrido[1,2-a]pyrimidin-4-one, 2-(5-fluoro-6-methoxypyridin-3-yl)-7-[(3S)-3-methylpiperazin-1-yl]-4H-pyrido[1,2-a]pyrimidin-4-one, 7-[(3R,5S)-3,5-dimethylpiperazin-1-yl]-2-(5-fluoro-6-methoxypyridin-3-yl)-4H-pyrido[1,2-a]pyrimidin-4-one, 7-[(3R,5S)-3,5-dimethylpiperazin-1-yl]-2-(2-methyl-1,3-benzothiazol-5-yl)-4H-pyrido[1,2-a]pyrimidin-4-one, 2-(2-methyl-1,3-benzothiazol-5-yl)-7-(4-methylpiperazin-1-yl)-4H-pyrido[1,2-a]pyrimidin-4-one, 2-(2-methyl-1,3 benzothiazol-5-yl)-7-(4-methyl-1,4-diazepan-1-yl)-4H-pyrido[1,2-a]pyrimidin-4-one, 7-[(8aS)-hexahydropyrrolo[1,2-a]pyrazin-2 (1H)-yl]-2-(2-methyl-1,3-benzothiazol-5-yl)-4H-pyrido[1,2-a]pyrimidin-4-one, 2-(4-methyl-1H-imidazol-1-yl)-7-(piperazin-1-yl)-4H-pyrido[1,2-a]pyrimidin-4-one, 2-(4-methyl-1H-imidazol-1-yl)-7-[(3S)-3-methylpiperazin-1-yl]-4H-pyrido[1,2-a]pyrimidin-4-one, 2-(5-fluoro-6-methoxypyridin-3-yl)-7-(piperazin-1-yl)-4H-pyrido[1,2-a]pyrimidin-4-one, 7-[(3S)-3-methylpiperazin-1-yl]-2-(1-methyl-1H-pyrazol-4-yl)-4H-pyrido[1,2-a]pyrimidin-4-one, 2-(1-methyl-1H-pyrazol-4-yl)-7-(piperazin-1-yl)-4H-pyrido[1,2-a]pyrimidin-4-one, 2-(2-methyl-1,3-benzoxazol-6-yl)-7-[(3R)-3-methylpiperazin-1-yl]-4H-pyrido[1,2-a]pyrimidin-4-one, 7-(3,3-dimethylpiperazin-1-yl)-2-(2-methyl-1,3-benzoxazol-6-yl)-4H-pyrido[1,2-a]pyrimidin-4-one, 7-(1,4-diazepan-1-yl)-2-(2-methyl-1,3-benzoxazol-6-yl)-4H-pyrido[1,2-a]pyrimidin-4-one, 2-(2-methyl-1,3-benzoxazol-6-yl)-7-(4-methyl-1,4-diazepan-1-yl)-4H-pyrido[1,2-a]pyrimidin-4-one, 7-[(8aS)-hexahydropyrrolo[1,2-a]pyrazin-2 (1H)-yl]-2-(2-methyl-1,3-benzoxazol-6-yl)-4H-pyrido[1,2-a]pyrimidin-4-one, 7-[(8aR)-hexahydropyrrolo[1,2-a]pyrazin-2 (1H)-yl]-2-(2-methyl-1,3-benzoxazol-6-yl)-4H-pyrido[1,2-a]pyrimidin-4-one, 2-(4,5-dimethoxypyridin-2-yl)-7-(piperazin-1-yl)-4H-pyrido[1,2-a]pyrimidin-4-one, 7-(4-ethylpiperazin-1-yl)-2-(2-methyl-1,3-benzoxazol-6-yl)-4H-pyrido[1,2-a]pyrimidin-4-one, 2-(8-fluoro-2-methylimidazo[1,2-a]pyridin-6-yl)-7-(piperazin-1-yl)-4H-pyrido[1,2-a]pyrimidin-4-one, 7-[(3R,5S)-3,5-dimethylpiperazin-1-yl]-2-(8-fluoro-2-methylimidazo[1,2-a]pyridin-6-yl)-4H-pyrido[1,2-a]pyrimidin-4-one, 2-(8-fluoro-2-methylimidazo[1,2-a]pyridin-6-yl)-7-(4-methyl-1,4-diazepan-1-yl)-4H-pyrido[1,2-a]pyrimidin-4-one, 2-(2-methyl-1,3-benzothiazol-6-yl)-7-(piperazin-1-yl)-4H-pyrido[1,2-a]pyrimidin-4-one, 2-(2-methyl-1,3-benzothiazol-6-yl)-7-[(3S)-3-methylpiperazin-1-yl]-4H-pyrido[1,2-a]pyrimidin-4-one, 2-(6-methoxypyridin-2-yl)-7-(piperazin-1-yl)-4H-pyrido[1,2-a]pyrimidin-4-one, 7-(piperazin-1-yl)-2-(pyridin-3-yl)-4H-pyrido[1,2-a]pyrimidin-4-one, 2-(5-methoxypyridin-3-yl)-7-(piperazin-1-yl)-4H-pyrido[1,2-a]pyrimidin-4-one, 7-[(3 S)-3,4-dimethylpiperazin-1-yl]-2-(2-methyl-1,3-benzoxazol-6-yl)-4H-pyrido[1,2-a]pyrimidin-4-one, 7-[4-(dimethylamino) piperidin-1-yl]-2-(2-methyl-1,3-benzoxazol-6-yl)-4H-pyrido[1,2-a]pyrimidin-4-one, 7-[(3S)-3-(dimethylamino)pyrrolidin-1-yl]-2-(2-methyl-1,3-benzoxazol-6-yl)-4H-pyrido[1,2-a]pyrimidin-4-one, 7-(4-aminopiperidin-1-yl)-2-(2-methyl-1,3-benzoxazol-6-yl)-4H-pyrido[1,2-a]pyrimidin-4-one, 7-[(3aR,6aS)-hexahydropyrrolo[3,4-c]pyrrol-2 (1H)-yl]-2-(2-methyl-1,3-benzoxazol-6-yl)-4H-pyrido[1,2-a]pyrimidin-4-one, 7-[(3R)-3,4-dimethylpiperazin-1-yl]-2-(2-methyl-1,3-benzoxazol-6-yl)-4H-pyrido[1,2-a]pyrimidin-4-one, 7-(1,4-diazepan-1-yl)-2-(2-methyl-1,3-benzothiazol-6-yl)-4H-pyrido[1,2-a]pyrimidin-4-one, 2-(2-methyl-1,3-benzothiazol-6-yl)-7-(4-methyl-1,4-diazepan-1-yl)-4H-pyrido[1,2-a]pyrimidin-4-one, 7-[(8aS)-hexahydropyrrolo[1,2-a]pyrazin-2 (1H)-yl]-2-(2-methyl-1,3-benzothiazol-6-yl)-4H-pyrido[1,2-a]pyrimidin-4-one, 2-(2-methyl-1,3-benzothiazol-6-yl)-7-(4-methylpiperazin-1-yl)-4H-pyrido[1,2-a]pyrimidin-4-one, 7-[(3S)-3,4-dimethylpiperazin-1-yl]-2-(4,6-dimethylpyrazolo[1,5-a]pyrazin-2-yl)-4H-pyrido[1,2-a]pyrimidin-4-one, 2-(4,6-dimethylpyrazolo[1,5-a]pyrazin-2-yl)-7-[(3R,5S)-3,4,5-trimethylpiperazin-1-yl]-4H-pyrido[1,2-a]pyrimidin-4-one, 7-[(3R)-3-(dimethylamino)pyrrolidin-1-yl]-2-(2-methyl-1,3-benzoxazol-6-yl)-4H-pyrido[1,2-a]pyrimidin-4-one, 2-(2-ethyl-1,3-benzoxazol-6-yl)-7-(piperazin-1-yl)-4H-pyrido[1,2-a]pyrimidin-4-one, 2-(2-ethyl-1,3-benzoxazol-6-yl)-7-[(3S)-3-methylpiperazin-1-yl]-4H-pyrido[1,2-a]pyrimidin-4-one, 2-(2-methyl-1,3-benzoxazol-6-yl)-7-[(3aR,6aS)-5-methylhexahydropyrrolo[3,4-c]pyrrol-2 (1H)-yl]-4H-pyrido[1,2-a]pyrimidin-4-one, 2-(8-fluoro-2-methylimidazo[1,2-a]pyridin-6-yl)-7-(4-methylpiperazin-1-yl)-4H-pyrido[1,2-a]pyrimidin-4-one, 2-(8-fluoro-2-methylimidazo[1,2-a]pyridin-6-yl)-7-[(3R)-3-methylpiperazin-1-yl]-4H-pyrido[1,2-a]pyrimidin-4-one, 2-(8-fluoro-2-methylimidazo[1,2-a]pyridin-6-yl)-7-[(8aS)-hexahydropyrrolo[1,2-a]pyrazin-2 (1H)-yl]-4H-pyrido[1,2-a]pyrimidin-4-one, 2-(8-fluoro-2-methylimidazo[1,2-a]pyridin-6-yl)-7-[(8aR)-hexahydropyrrolo[1,2-a]pyrazin-2 (1H)-yl]-4H-pyrido[1,2-a]pyrimidin-4-one, 7-(4-aminopiperidin-1-yl)-2-(8-fluoro-2-methylimidazo[1,2-a]pyridin-6-yl)-4H-pyrido[1,2-a]pyrimidin-4-one, 7-[(3R)-3-(dimethylamino)pyrrolidin-1-yl]-2-(8-fluoro-2-methylimidazo[1,2-a]pyridin-6-yl)-4H-pyrido[1,2-a]pyrimidin-4-one, 7-[(3S)-3-(dimethylamino)pyrrolidin-1-yl]-2-(8-fluoro-2-methylimidazo[1,2-a]pyridin-6-yl)-4H-pyrido[1,2-a]pyrimidin-4-one, 7-(3,3-dimethylpiperazin-1-yl)-2-(2-methyl-1,3-benzothiazol-6-yl)-4H-pyrido[1,2-a]pyrimidin-4-one, 7-[(8aR)-hexahydropyrrolo[1,2-a]pyrazin-2 (1H)-yl]-2-(2-methyl-1,3-benzothiazol-6-yl)-4H-pyrido[1,2-a]pyrimidin-4-one, 7-[4-(dimethylamino) piperidin-1-yl]-2-(2-methyl-1,3-benzothiazol-6-yl)-4H-pyrido[1,2-a]pyrimidin-4-one, 2-(2-methyl-1,3-benzothiazol-6-yl)-7-(piperidin-4-yloxy)-4H-pyrido[1,2-a]pyrimidin-4-one, 2-(8-chloro-2-methylimidazo[1,2-a]pyridin-6-yl)-7-(piperazin-1-yl)-4H-pyrido[1,2-a]pyrimidin-4-one, 2-(8-chloro-2-methylimidazo[1,2-a]pyridin-6-yl)-7-[(3R,5S)-3,5-dimethylpiperazin-1-yl]-4H-pyrido[1,2-a]pyrimidin-4-one, 2-(8-chloro-2-methylimidazo[1,2-a]pyridin-6-yl)-7-(4-methyl-1,4-diazepan-1-yl)-4H-pyrido[1,2-a]pyrimidin-4-one, 2-(8-chloro-2-methylimidazo[1,2-a]pyridin-6-yl)-7-[(3R)-3-methylpiperazin-1-yl]-4H-pyrido[1,2-a]pyrimidin-4-one, 2-(8-chloro-2-methylimidazo[1,2-a]pyridin-6-yl)-7-[(3S)-3-methylpiperazin-1-yl]-4H-pyrido[1,2-a]pyrimidin-4-one, 2-(8-chloro-2-methylimidazo[1,2-a]pyridin-6-yl)-7-[(8aR)-hexahydropyrrolo[1,2-a]pyrazin-2 (1H)-yl]-4H-pyrido[1,2-a]pyrimidin-4-one, 2-(8-chloro-2-methylimidazo[1,2-a]pyridin-6-yl)-7-[(8aS)-hexahydropyrrolo[1,2-a]pyrazin-2 (1H)-yl]-4H-pyrido[1,2-a]pyrimidin-4-one, 7-[(3S)-3,4-dimethylpiperazin-1-yl]-2-(8-fluoro-2-methylimidazo[1,2-a]pyridin-6-yl)-4H-pyrido[1,2-a]pyrimidin-4-one, 7-[(3R)-3,4-dimethylpiperazin-1-yl]-2-(8-fluoro-2-methylimidazo[1,2-a]pyridin-6-yl)-4H-pyrido[1,2-a]pyrimidin-4-one, 7-[4-(dimethylamino) piperidin-1-yl]-2-(8-fluoro-2-methylimidazo[1,2-a]pyridin-6-yl)-4H-pyrido[1,2-a]pyrimidin-4-one, 2-(4-fluoro-2-methyl-1,3-benzoxazol-6-yl)-7-(1,2,3,6-tetrahydropyridin-4-yl)-4H-pyrido[1,2-a]pyrimidin-4-one, 2-(4-fluoro-2-methyl-1,3-benzoxazol-6-yl)-7-(1-methyl-1,2, 3,6-tetrahydropyridin-4-yl)-4H-pyrido[1,2-a]pyrimidin-4-one, 2-(2-methyl-1,3-benzoxazol-6-yl)-7-(1,2,3,6-tetrahydropyridin-4-yl)-4H-pyrido[1,2-a]pyrimidin-4-one, 2-(2-methyl-1,3-benzoxazol-6-yl)-7-(1-methyl-1,2,3,6-tetrahydropyridin-4-yl)-4H-pyrido[1,2-a]pyrimidin-4-one, 7-(1-ethyl-1,2,3,6-tetrahydropyridin-4-yl)-2-(2-methyl-1,3-benzoxazol-6-yl)-4H-pyrido[1,2-a]pyrimidin-4-one, 2-(4-fluoro-2-methyl-1,3-benzoxazol-6-yl)-7-(piperazin-1-yl)-4H-pyrido[1,2-a]pyrimidin-4-one, 2-(4-fluoro-2-methyl-1,3-benzoxazol-6-yl)-7-[(3S)-3-methylpiperazin-1-yl]-4H-pyrido[1,2-a]pyrimidin-4-one, 7-[(3S)-3,4-dimethylpiperazin-1-yl]-2-(4-fluoro-2-methyl-1,3-benzoxazol-6-yl)-4H-pyrido[1,2-a]pyrimidin-4-one, 2-(4-fluoro-2-methyl-1,3-benzoxazol-6-yl)-7-(4-methylpiperazin-1-yl)-4H-pyrido[1,2-a]pyrimidin-4-one, 7-(4-ethylpiperazin-1-yl)-2-(4-fluoro-2-methyl-1,3-benzoxazol-6-yl)-4H-pyrido[1,2-a]pyrimidin-4-one, 2-(4-fluoro-2-methyl-1,3-benzoxazol-6-yl)-7-(4-propylpiperazin-1-yl)-4H-pyrido[1,2-a]pyrimidin-4-one, 7-(1-ethyl-1,2,3,6-tetrahydropyridin-4-yl)-2-(4-fluoro-2-methyl-1,3-benzoxazol-6-yl)-4H-pyrido[1,2-a]pyrimidin-4-one, 2-(4-fluoro-2-methyl-1,3-benzoxazol-6-yl)-7-(1-propyl-1,2,3,6-tetrahydropyridin-4-yl)-4H-pyrido[1,2-a]pyrimidin-4-one, 2-(8-chloro-2-methylimidazo[1,2-a]pyridin-6-yl)-7-[(3 S)-3,4-dimethylpiperazin-1-yl]-4H-pyrido[1,2-a]pyrimidin-4-one, 2-(4,6-dimethylpyrazolo[1,5-a]pyrazin-2-yl)-7-(4-methyl-1,4-diazepan-1-yl)-4H-pyrido[1,2-a]pyrimidin-4-one, 2-(4,6-dimethylpyrazolo[1,5-a]pyrazin-2-yl)-7-[(3R)-3-methylpiperazin-1-yl]-4H-pyrido[1,2-a]pyrimidin-4-one, 2-(4,6-dimethylpyrazolo[1,5-a]pyrazin-2-yl)-7-[(8aS)-hexahydropyrrolo[1,2-a]pyrazin-2 (1H)-yl]-4H-pyrido[1,2-a]pyrimidin-4-one, 2-(4,6-dimethylpyrazolo[1,5-a]pyrazin-2-yl)-7-[(8aR)-hexahydropyrrolo[1,2-a]pyrazin-2 (1H)-yl]-4H-pyrido[1,2-a]pyrimidin-4-one, 2-(4,6-dimethylpyrazolo[1,5-a]pyrazin-2-yl)-7-(4-ethylpiperazin-1-yl)-4H-pyrido[1,2-a]pyrimidin-4-one, 7-[4-(dimethylamino) piperidin-1-yl]-2-(4,6-dimethylpyrazolo[1,5-a]pyrazin-2-yl)-4H-pyrido[1,2-a]pyrimidin-4-one, 7-(3,3-dimethylpiperazin-1-yl)-2-(4,6-dimethylpyrazolo[1,5-a]pyrazin-2-yl)-4H-pyrido[1,2-a]pyrimidin-4-one, 7-(4-cyclopropylpiperazin-1-yl)-2-(4,6-dimethylpyrazolo[1,5-a]pyrazin-2-yl)-4H-pyrido[1,2-a]pyrimidin-4-one, 2-(4,6-dimethylpyrazolo[1,5-a]pyrazin-2-yl)-7-(1,2,3,6-tetrahydropyridin-4-yl)-4H-pyrido[1,2-a]pyrimidin-4-one, 2-(4,6-dimethylpyrazolo[1,5-a]pyrazin-2-yl)-7-[(3R)-4-ethyl-3-methylpiperazin-1-yl]-4H-pyrido[1,2-a]pyrimidin-4-one, 7-[4-(dimethylamino) piperidin-1-yl]-2-(2-methyl-1,3-benzothiazol-5-yl)-4H-pyrido[1,2-a]pyrimidin-4-one, 7-(4-aminopiperidin-1-yl)-2-(2-methyl-1,3-benzothiazol-5-yl)-4H-pyrido[1,2-a]pyrimidin-4-one, 7-[(3 aR,6aS)-hexahydropyrrolo[3,4-c]pyrrol-2 (1H)-yl]-2-(2-methyl-1,3-benzothiazol-5-yl)-4H-pyrido[1,2-a]pyrimidin-4-one, 2-(4,6-dimethylpyrazolo[1,5-a]pyrazin-2-yl)-7-(1-methyl-1,2,3,6-tetrahydropyridin-4-yl)-4H-pyrido[1,2-a]pyrimidin-4-one, 2-(4,6-dimethylpyrazolo[1,5-a]pyrazin-2-yl)-7-(1-ethyl-1,2,3,6-tetrahydropyridin-4-yl)-4H-pyrido[1,2-a]pyrimidin-4-one, 2-(4,6-dimethylpyrazolo[1,5-a]pyrazin-2-yl)-7-(1-propyl-1,2,3,6-tetrahydropyridin-4-yl)-4H-pyrido[1,2-a]pyrimidin-4-one, 7-(1-cyclopropyl-1,2,3,6-tetrahydropyridin-4-yl)-2-(4,6-dimethylpyrazolo[1,5-a]pyrazin-2-yl)-4H-pyrido[1,2-a]pyrimidin-4-one, 2-(4,6-dimethylpyrazolo[1,5-a]pyrazin-2-yl)-7-[1-(propan-2-yl)-1,2,3,6-tetrahydropyridin-4-yl]-4H-pyrido[1,2-a]pyrimidin-4-one, 7-(1-cyclobutyl-1,2,3,6-tetrahydropyridin-4-yl)-2-(4,6-dimethylpyrazolo[1,5-a]pyrazin-2-yl)-4H-pyrido[1,2-a]pyrimidin-4-one, 2-(4,6-dimethylpyrazolo[1,5-a]pyrazin-2-yl)-7-[1-(oxetan-3-yl)-1,2,3,6-tetrahydropyridin-4-yl]-4H-pyrido[1,2-a]pyrimidin-4-one, 2-(8-fluoro-2-methylimidazo[1,2-a]pyridin-6-yl)-7-[4-(propan-2-yl) piperazin-1-yl]-4H-pyrido[1,2-a]pyrimidin-4-one, 2-(8-chloro-2-methylimidazo[1,2-a]pyridin-6-yl)-7-[4-(propan-2-yl) piperazin-1-yl]-4H-pyrido[1,2-a]pyrimidin-4-one, 7-(4-aminopiperidin-1-yl)-2-(2-methyl-1,3-benzothiazol-6-yl)-4H-pyrido[1,2-a]pyrimidin-4-one, 7-(3-aminopyrrolidin-1-yl)-2-(2-methyl-1,3-benzothiazol-6-yl)-4H-pyrido[1,2-a]pyrimidin-4-one, 7-[(3R)-3-(dimethylamino)pyrrolidin-1-yl]-2-(2-methyl-1,3-benzothiazol-6-yl)-4H-pyrido[1,2-a]pyrimidin-4-one, 2-(2-methyl-1,3-benzothiazol-6-yl)-7-(1,2,3,6-tetrahydropyridin-4-yl)-4H-pyrido[1,2-a]pyrimidin-4-one, 2-(8-fluoro-2-methylimidazo[1,2-a]pyridin-6-yl)-7-[4-(2-methoxyethyl) piperazin-1-yl]-4H-pyrido[1,2-a]pyrimidin-4-one, 2-(2-methyl-1,3-benzothiazol-6-yl)-7-(1-methyl-1,2,3,6-tetrahydropyridin-4-yl)-4H-pyrido[1,2-a]pyrimidin-4-one, 7-[1-(2-hydroxyethyl)-1,2,3,6-tetrahydropyridin-4-yl]-2-(2-methyl-1,3-benzothiazol-6-yl)-4H-pyrido[1,2-a]pyrimidin-4-one, 2-(2-methyl-1,3-benzothiazol-6-yl)-7-[1-(propan-2-yl)-1,2,3,6-tetrahydropyridin-4-yl]-4H-pyrido[1,2-a]pyrimidin-4-one, 7-(1-cyclopropyl-1,2,3,6-tetrahydropyridin-4-yl)-2-(2-methyl-1,3-benzothiazol-6-yl)-4H-pyrido[1,2-a]pyrimidin-4-one, 7-(1-ethyl-1,2,3,6-tetrahydropyridin-4-yl)-2-(2-methyl-1,3-benzothiazol-6-yl)-4H-pyrido[1,2-a]pyrimidin-4-one, 7-[(3R)-3,4-dimethylpiperazin-1-yl]-2-(4,6-dimethylpyrazolo[1,5-a]pyrazin-2-yl)-4H-pyrido[1,2-a]pyrimidin-4-one, 7-[(1S,4S)-2,5-diazabicyclo[2.2.1]hept-2-yl]-2-(4,6-dimethylpyrazolo[1,5-a]pyrazin-2-yl)-4H-pyrido[1,2-a]pyrimidin-4-one, 2-(4,6-dimethylpyrazolo[1,5-a]pyrazin-2-yl)-7-[(1S,4S)-5-methyl-2,5-diazabicyclo[2.2.1]hept-2-yl]-4H-pyrido[1,2-a]pyrimidin-4-one, 2-(4,6-dimethylpyrazolo[1,5-a]pyrazin-2-yl)-7-[(1 S,4S)-5-ethyl-2,5-diazabicyclo[2.2.1]hept-2-yl]-4H-pyrido[1,2-a]pyrimidin-4-one, 7-(4-cyclopropylpiperazin-1-yl)-2-(8-fluoro-2-methylimidazo[1,2-a]pyridin-6-yl)-4H-pyrido[1,2-a]pyrimidin-4-one, 2-(6-methylpyrazolo[1,5-a]pyrazin-2-yl)-7-(piperazin-1-yl)-4H-pyrido[1,2-a]pyrimidin-4-one, 2-(2-methyl-1,3-benzothiazol-6-yl)-7-(piperidin-4-yl)-4H-pyrido[1,2-a]pyrimidin-4-one, 2-(4,6-dimethylpyrazolo[1,5-a]pyrazin-2-yl)-7-[4-(pyrrolidin-1-yl) piperidin-1-yl]-4H-pyrido[1,2-a]pyrimidin-4-one, 7-(1,4′-bipiperidin-1′-yl)-2-(4,6-dimethylpyrazolo[1,5-a]pyrazin-2-yl)-4H-pyrido[1,2-a]pyrimidin-4-one, 2-(4,6-dimethylpyrazolo[1,5-a]pyrazin-2-yl)-7-[4-(4-methylpiperazin-1-yl) piperidin-1-yl]-4H-pyrido[1,2-a]pyrimidin-4-one, 2-(4,6-dimethylpyrazolo[1,5-a]pyrazin-2-yl)-7-[4-(morpholin-4-yl) piperidin-1-yl]-4H-pyrido[1,2-a]pyrimidin-4-one, 2-(2-methylimidazo[1,2-a]pyridin-6-yl)-7-(1,2,3,6-tetrahydropyridin-4-yl)-4H-pyrido[1,2-a]pyrimidin-4-one, 2-(2-methylimidazo[1,2-a]pyridin-6-yl)-7-(1-methyl-1,2,3,6-tetrahydropyridin-4-yl)-4H-pyrido[1,2-a]pyrimidin-4-one, 7-(1-ethyl-1,2,3,6-tetrahydropyridin-4-yl)-2-(2-methylimidazo[1,2-a]pyridin-6-yl)-4H-pyrido[1,2-a]pyrimidin-4-one, 7-[4-(dimethylamino) piperidin-1-yl]-2-(2-methylimidazo[1,2-a]pyridin-6-yl)-4H-pyrido[1,2-a]pyrimidin-4-one, 2-(2-methylimidazo[1,2-a]pyridin-6-yl)-7-[4-(pyrrolidin-1-yl) piperidin-1-yl]-4H-pyrido[1,2-a]pyrimidin-4-one, 2-(2-methylimidazo[1,2-a]pyridin-6-yl)-7-(piperidin-4-yl)-4H-pyrido[1,2-a]pyrimidin-4-one, 2-(2-methyl-1,3-benzothiazol-6-yl)-7-(1-methylpiperidin-4-yl)-4H-pyrido[1,2-a]pyrimidin-4-one, 7-(4-methylpiperazin-1-yl)-2-(6-methylpyrazolo[1,5-a]pyrazin-2-yl)-4H-pyrido[1,2-a]pyrimidin-4-one, 7-[(3S)-3-methylpiperazin-1-yl]-2-(6-methylpyrazolo[1,5-a]pyrazin-2-yl)-4H-pyrido[1,2-a]pyrimidin-4-one, 7-[(3R,5S)-3,5-dimethylpiperazin-1-yl]-2-(6-methylpyrazolo[1,5-a]pyrazin-2-yl)-4H-pyrido[1,2-a]pyrimidin-4-one, 2-(1-methyl-1H-indazol-5-yl)-7-(1,2,3,6-tetrahydropyridin-4-yl)-4H-pyrido[1,2-a]pyrimidin-4-one, 2-[6-(dimethylamino)pyridin-3-yl]-7-(1,2,3,6-tetrahydropyridin-4-yl)-4H-pyrido[1,2-a]pyrimidin-4-one, 7-[4-(diethylamino) piperidin-1-yl]-2-(2-methylimidazo[1,2-a]pyridin-6-yl)-4H-pyrido[1,2-a]pyrimidin-4-one, 2-(2-methylimidazo[1,2-a]pyridin-6-yl)-7-(1-methylpiperidin-4-yl)-4H-pyrido[1,2-a]pyrimidin-4-one, 2-(1-methyl-1H-indazol-5-yl)-7-(1-methyl-1,2,3,6-tetrahydropyridin-4-yl)-4H-pyrido[1,2-a]pyrimidin-4-one, 2-[6-(dimethylamino)pyridin-3-yl]-7-(1-methyl-1,2,3,6-tetrahydropyridin-4-yl)-4H-pyrido[1,2-a]pyrimidin-4-one, 7-[4-(diethylamino) piperidin-1-yl]-2-(4,6-dimethylpyrazolo[1,5-a]pyrazin-2-yl)-4H-pyrido[1,2-a]pyrimidin-4-one, 2-(4,6-dimethylpyrazolo[1,5-a]pyrazin-2-yl)-7-(piperidin-4-yl)-4H-pyrido[1,2-a]pyrimidin-4-one, 2-(4,6-dimethylpyrazolo[1,5-a]pyrazin-2-yl)-7-(1-methylpiperidin-4-yl)-4H-pyrido[1,2-a]pyrimidin-4-one, 2-(4,6-dimethylpyrazolo[1,5-a]pyrazin-2-yl)-7-(1-ethylpiperidin-4-yl)-4H-pyrido[1,2-a]pyrimidin-4-one, 2-(4,6-dimethylpyrazolo[1,5-a]pyrazin-2-yl)-9-methyl-7-(4-methylpiperazin-1-yl)-4H-pyrido[1,2-a]pyrimidin-4-one, 2-(4,6-dimethylpyrazolo[1,5-a]pyrazin-2-yl)-7-[(8aR)-hexahydropyrrolo[1,2-a]pyrazin-2 (1H)-yl]-9-methyl-4H-pyrido[1,2-a]pyrimidin-4-one, 7-[4-(dimethylamino) piperidin-1-yl]-2-(4,6-dimethylpyrazolo[1,5-a]pyrazin-2-yl)-9-methyl-4H-pyrido[1,2-a]pyrimidin-4-one, 7-(1-ethyl-1,2,3,6-tetrahydropyridin-4-yl)-2-(1-methyl-1H-indazol-5-yl)-4H-pyrido[1,2-a]pyrimidin-4-one, 2-(1-methyl-1H-indazol-5-yl)-7-(1-propyl-1,2,3,6-tetrahydropyridin-4-yl)-4H-pyrido[1,2-a]pyrimidin-4-one, 2-[6-(dimethylamino)pyridin-3-yl]-7-(piperidin-4-yl)-4H-pyrido[1,2-a]pyrimidin-4-one, 7-(4-ethylpiperazin-1-yl)-2-(6-methylpyrazolo[1,5-a]pyrazin-2-yl)-4H-pyrido[1,2-a]pyrimidin-4-one, 7-[(8aR)-hexahydropyrrolo[1,2-a]pyrazin-2 (1H)-yl]-2-(6-methylpyrazolo[1,5-a]pyrazin-2-yl)-4H-pyrido[1,2-a]pyrimidin-4-one, 7-[4-(dimethylamino) piperidin-1-yl]-2-(6-methylpyrazolo[1,5-a]pyrazin-2-yl)-4H-pyrido[1,2-a]pyrimidin-4-one, 7-[4-(2-hydroxyethyl) piperazin-1-yl]-2-(6-methylpyrazolo[1,5-a]pyrazin-2-yl)-4H-pyrido[1,2-a]pyrimidin-4-one, 2-(2-methylimidazo[1,2-a]pyridin-6-yl)-7-(1-propylpiperidin-4-yl)-4H-pyrido[1,2-a]pyrimidin-4-one, 7-[1-(2-hydroxyethyl)-1,2,3,6-tetrahydropyridin-4-yl]-2-(1-methyl-1H-indazol-5-yl)-4H-pyrido[1,2-a]pyrimidin-4-one, 7-[(3R)-3-methylpiperazin-1-yl]-2-(6-methylpyrazolo[1,5-a]pyrazin-2-yl)-4H-pyrido[1,2-a]pyrimidin-4-one, 2-(6-methylpyrazolo[1,5-a]pyrazin-2-yl)-7-(1,2,3,6-tetrahydropyridin-4-yl)-4H-pyrido[1,2-a]pyrimidin-4-one, 2-(2-methyl-2H-indazol-5-yl)-7-(1,2,3,6-tetrahydropyridin-4-yl)-4H-pyrido[1,2-a]pyrimidin-4-one, 2-(1-methyl-1H-indazol-5-yl)-7-(1-methylpiperidin-4-yl)-4H-pyrido[1,2-a]pyrimidin-4-one, 2-(2-methyl-2H-indazol-5-yl)-7-(1-methyl-1,2,3,6-tetrahydropyridin-4-yl)-4H-pyrido[1,2-a]pyrimidin-4-one, 7-(1-ethyl-1,2,3,6-tetrahydropyridin-4-yl)-2-(2-methyl-2H-indazol-5-yl)-4H-pyrido[1,2-a]pyrimidin-4-one, 7-(1-ethylpiperidin-4-yl)-2-(2-methylimidazo[1,2-a]pyridin-6-yl)-4H-pyrido[1,2-a]pyrimidin-4-one, 2-(1,3-dimethylpyrrolo[1,2-a]pyrazin-7-yl)-7-[1-(propan-2-yl)-1,2,3,6-tetrahydropyridin-4-yl]-4H-pyrazino[1,2-a]pyrimidin-4-one, 2-(4,6-dimethylpyrazolo[1,5-a]pyrazin-2-yl)-9-methyl-7-(1,2,3,6-tetrahydropyridin-4-yl)-4H-pyrido[1,2-a]pyrimidin-4-one, 2-(4,6-dimethylpyrazolo[1,5-a]pyrazin-2-yl)-9-methyl-7-(1-methyl-1,2,3,6-tetrahydropyridin-4-yl)-4H-pyrido[1,2-a]pyrimidin-4-one, 2-(4,6-dimethylpyrazolo[1,5-a]pyrazin-2-yl)-7-(1-ethyl-1,2,3,6-tetrahydropyridin-4-yl)-9-methyl-4H-pyrido[1,2-a]pyrimidin-4-one, 7-(1-cyclopropyl-1,2,3,6-tetrahydropyridin-4-yl)-2-(4,6-dimethylpyrazolo[1,5-a]pyrazin-2-yl)-9-methyl-4H-pyrido[1,2-a]pyrimidin-4-one, 2-(6-methylpyrazolo[1,5-a]pyrazin-2-yl)-7-(1-methyl-1,2, 3,6-tetrahydropyridin-4-yl)-4H-pyrido[1,2-a]pyrimidin-4-one, 7-(1-ethyl-1,2,3,6-tetrahydropyridin-4-yl)-2-(6-methylpyrazolo[1,5-a]pyrazin-2-yl)-4H-pyrido[1,2-a]pyrimidin-4-one, 2-(5,7-dimethylfuro [2,3-c]pyridin-2-yl)-7-(piperazin-1-yl)-4H-pyrido[1,2-a]pyrimidin-4-one, 2-(5,7-dimethylfuro [2,3-c]pyridin-2-yl)-7-(4-methylpiperazin-1-yl)-4H-pyrido[1,2-a]pyrimidin-4-one, 2-(5,7-dimethylfuro [2,3-c]pyridin-2-yl)-7-[(3S)-3-methylpiperazin-1-yl]-4H-pyrido[1,2-a]pyrimidin-4-one, 2-(1-methyl-1H-indazol-5-yl)-7-(4-methylpiperazin-1-yl)-4H-pyrido[1,2-a]pyrimidin-4-one, 2-(1-methyl-1H-indazol-5-yl)-7-(piperazin-1-yl)-4H-pyrido[1,2-a]pyrimidin-4-one, 7-[4-(dimethylamino) piperidin-1-yl]-2-(1-methyl-1H-indazol-5-yl)-4H-pyrido[1,2-a]pyrimidin-4-one, 7-(4-methyl-1,4-diazepan-1-yl)-2-(1-methyl-1H-indazol-5-yl)-4H-pyrido[1,2-a]pyrimidin-4-one, 7-(1-ethyl-1,2,3,6-tetrahydropyridin-4-yl)-2-(2-methyl-1,3-benzoxazol-6-yl)-4H-pyrimido[1,2-b]pyridazin-4-one, 2-(8-chloro-2-methylimidazo[1,2-a]pyridin-6-yl)-7-(4-ethylpiperazin-1-yl)-4H-pyrido[1,2-a]pyrimidin-4-one, 2-(8-chloro-2-methylimidazo[1,2-a]pyridin-6-yl)-7-[(3R)-4-ethyl-3-methylpiperazin-1-yl]-4H-pyrido[1,2-a]pyrimidin-4-one, 2-(8-chloro-2-methylimidazo[1,2-a]pyridin-6-yl)-7-[(3S)-4-ethyl-3-methylpiperazin-1-yl]-4H-pyrido[1,2-a]pyrimidin-4-one, 2-(8-chloro-2-methylimidazo[1,2-a]pyridin-6-yl)-7-[(3R)-3-methyl-4-(propan-2-yl) piperazin-1-yl]-4H-pyrido[1,2-a]pyrimidin-4-one, 2-(8-chloro-2-methylimidazo[1,2-a]pyridin-6-yl)-7-[(3 S)-3-methyl-4-(propan-2-yl) piperazin-1-yl]-4H-pyrido[1,2-a]pyrimidin-4-one, 2-(4,6-dimethylpyrazolo[1,5-a]pyrazin-2-yl)-7-(4-ethylpiperazin-1-yl)-9-methyl-4H-pyrido[1,2-a]pyrimidin-4-one, 2-(4,6-dimethylpyrazolo[1,5-a]pyrazin-2-yl)-9-methyl-7-[(3R)-3-methylpiperazin-1-yl]-4H-pyrido[1,2-a]pyrimidin-4-one, 7-[(3R)-3,4-dimethylpiperazin-1-yl]-2-(4,6-dimethylpyrazolo[1,5-a]pyrazin-2-yl)-9-methyl-4H-pyrido[1,2-a]pyrimidin-4-one, 2-(4,6-dimethylpyrazolo[1,5-a]pyrazin-2-yl)-7-[(3R)-4-ethyl-3-methylpiperazin-1-yl]-9-methyl-4H-pyrido[1,2-a]pyrimidin-4-one, 2-(4,6-dimethylpyrazolo[1,5-a]pyrazin-2-yl)-7-(octahydro-5H-pyrrolo[3,2-c]pyridin-5-yl)-4H-pyrido[1,2-a]pyrimidin-4-one, 2-(8-chloro-2-methylimidazo[1,2-a]pyridin-6-yl)-7-(piperidin-4-yl)-4H-pyrido[1,2-a]pyrimidin-4-one, 2-(8-chloro-2-methylimidazo[1,2-a]pyridin-6-yl)-7-(1-methylpiperidin-4-yl)-4H-pyrido[1,2-a]pyrimidin-4-one, 2-(8-chloro-2-methylimidazo[1,2-a]pyridin-6-yl)-7-(1-ethylpiperidin-4-yl)-4H-pyrido[1,2-a]pyrimidin-4-one, 2-(8-chloro-2-methylimidazo[1,2-a]pyridin-6-yl)-7-[1-(propan-2-yl) piperidin-4-yl]-4H-pyrido[1,2-a]pyrimidin-4-one, 2-(4,6-dimethylpyrazolo[1, 5-a]pyrazin-2-yl)-9-methyl-7-(4-methyl-1,4-diazepan-1-yl)-4H-pyrido[1,2-a]pyrimidin-4-one, 2-(5,7-dimethylfuro [2,3-c]pyridin-2-yl)-7-[(3R,5 S)-3,5-dimethylpiperazin-1-yl]-4H-pyrido[1,2-a]pyrimidin-4-one, 7-[4-(dimethylamino) piperidin-1-yl]-2-(5,7-dimethylfuro [2,3-c]pyridin-2-yl)-4H-pyrido[1,2-a]pyrimidin-4-one, 2-(5,7-dimethylfuro [2,3-c]pyridin-2-yl)-7-[4-(2-hydroxyethyl) piperazin-1-yl]-4H-pyrido[1,2-a]pyrimidin-4-one, 2-(4,6-dimethylpyrazolo[1,5-a]pyrazin-2-yl)-7-[1-(2-hydroxyethyl) piperidin-4-yl]-4H-pyrido[1,2-a]pyrimidin-4-one, 2-(4,6-dimethylpyrazolo[1,5-a]pyrazin-2-yl)-7-[4-(2-hydroxyethyl) piperazin-1-yl]-4H-pyrido[1,2-a]pyrimidin-4-one, 2-(4,6-dimethylpyrazolo[1,5-a]pyrazin-2-yl)-7-[(3R)-4-(2-hydroxyethyl)-3-methylpiperazin-1-yl]-4H-pyrido[1,2-a]pyrimidin-4-one, 2-(8-chloro-2-methylimidazo[1,2-a]pyridin-6-yl)-7-[(3S)-4-(2-methoxyethyl)-3-methylpiperazin-1-yl]-4H-pyrido[1,2-a]pyrimidin-4-one, 2-(8-chloro-2-methylimidazo[1,2-a]pyridin-6-yl)-7- {(3S)-4-[2-(2-hydroxyethoxy)ethyl]-3-methylpiperazin-1-yl}-4H-pyrido[1,2-a]pyrimidin-4-one, 2-(8-chloro-2-methylimidazo[1,2-a]pyridin-6-yl)-7-[(3S)-4-cyclopropyl-3-methylpiperazin-1-yl]-4H-pyrido[1,2-a]pyrimidin-4-one, 2-(8-chloro-2-methylimidazo[1,2-a]pyridin-6-yl)-7-[(3S)-4-cyclobutyl-3-methylpiperazin-1-yl]-4H-pyrido[1,2-a]pyrimidin-4-one, 2-(8-chloro-2-methylimidazo[1,2-a]pyridin-6-yl)-7-[(3S)-4-(2-hydroxyethyl)-3-methylpiperazin-1-yl]-4H-pyrido[1,2-a]pyrimidin-4-one, 2-(8-fluoro-2-methylimidazo[1,2-a]pyridin-6-yl)-7-[(3 S)-4-(2-methoxyethyl)-3-methylpiperazin-1-yl]-4H-pyrido[1,2-a]pyrimidin-4-one, 2-(8-fluoro-2-methylimidazo[1,2-a]pyridin-6-yl)-7-[(3S)-4-(2-hydroxyethyl)-3-methylpiperazin-1-yl]-4H-pyrido[1,2-a]pyrimidin-4-one, 2-(8-fluoro-2-methylimidazo[1,2-a]pyridin-6-yl)-7- {(3 S)-4-[2-(2-hydroxyethoxy)ethyl]-3-methylpiperazin-1-yl}-4H-pyrido[1,2-a]pyrimidin-4-one, 2-(8-fluoro-2-methylimidazo[1,2-a]pyridin-6-yl)-7-[(3S)-3-methyl-4-(propan-2-yl) piperazin-1-yl]-4H-pyrido[1,2-a]pyrimidin-4-one, 7-[(3S)-4-cyclopropyl-3-methylpiperazin-1-yl]-2-(8-fluoro-2-methylimidazo[1,2-a]pyridin-6-yl)-4H-pyrido[1,2-a]pyrimidin-4-one, 7-[(3S)-4-cyclobutyl-3-methylpiperazin-1-yl]-2-(8-fluoro-2-methylimidazo[1,2-a]pyridin-6-yl)-4H-pyrido[1,2-a]pyrimidin-4-one, 7-(3,3-dimethylpiperazin-1-yl)-2-(4,6-dimethylpyrazolo[1,5-a]pyrazin-2-yl)-9-methyl-4H-pyrido[1,2-a]pyrimidin-4-one, 2-(1-methyl-1H-indazol-5-yl)-7-[(3R)-3-methylpiperazin-1-yl]-4H-pyrido[1,2-a]pyrimidin-4-one, 2-(4-ethyl-6-methylpyrazolo[1,5-a]pyrazin-2-yl)-7-(piperazin-1-yl)-4H-pyrido[1,2-a]pyrimidin-4-one, 2-(4-ethyl-6-methylpyrazolo[1,5-a]pyrazin-2-yl)-7-(4-methylpiperazin-1-yl)-4H-pyrido[1,2-a]pyrimidin-4-one, 2-(4-ethyl-6-methylpyrazolo[1,5-a]pyrazin-2-yl)-7-(4-ethylpiperazin-1-yl)-4H-pyrido[1,2-a]pyrimidin-4-one, 2-(4-ethyl-6-methylpyrazolo[1,5-a]pyrazin-2-yl)-7-[4-(2-hydroxyethyl) piperazin-1-yl]-4H-pyrido[1,2-a]pyrimidin-4-one, 7-[4-(dimethylamino) piperidin-1-yl]-2-(4-ethyl-6-methylpyrazolo[1,5-a]pyrazin-2-yl)-4H-pyrido[1,2-a]pyrimidin-4-one, 7-[4-(diethylamino) piperidin-1-yl]-2-(4-ethyl-6-methylpyrazolo[1,5-a]pyrazin-2-yl)-4H-pyrido[1,2-a]pyrimidin-4-one, 7-[(3R,5 S)-3,5-dimethylpiperazin-1-yl]-2-(1-methyl-1H-indazol-5-yl)-4H-pyrido[1,2-a]pyrimidin-4-one, 2-(1-methyl-1H-indazol-5-yl)-7-[(3S)-3-methylpiperazin-1-yl]-4H-pyrido[1,2-a]pyrimidin-4-one, 2-(2-methylimidazo[1,2-a]pyridin-6-yl)-7-[1-(propan-2-yl) piperidin-4-yl]-4H-pyrido[1,2-a]pyrimidin-4-one, 2-(2-methylimidazo[1,2-a]pyridin-7-yl)-7-(piperazin-1-yl)-4H-pyrido[1,2-a]pyrimidin-4-one, 2-(2-methylimidazo[1,2-a]pyridin-7-yl)-7-(piperidin-4-yl)-4H-pyrido[1,2-a]pyrimidin-4-one, 2-(2-methylimidazo[1,2-a]pyridin-7-yl)-7-(4-methylpiperazin-1-yl)-4H-pyrido[1,2-a]pyrimidin-4-one, 7-(4-ethylpiperazin-1-yl)-2-(2-methylimidazo[1,2-a]pyridin-7-yl)-4H-pyrido[1,2-a]pyrimidin-4-one, 2-(2-methylimidazo[1,2-a]pyridin-7-yl)-7-(1-methylpiperidin-4-yl)-4H-pyrido[1,2-a]pyrimidin-4-one, 7-(1-ethylpiperidin-4-yl)-2-(2-methylimidazo[1,2-a]pyridin-7-yl)-4H-pyrido[1,2-a]pyrimidin-4-one, 2-(4,6-dimethylpyrazolo[1,5-a]pyrazin-2-yl)-9-methyl-7-[(3 S)-3-methylpiperazin-1-yl]-4H-pyrido[1,2-a]pyrimidin-4-one, 7-[(3R,5S)-3,5-dimethylpiperazin-1-yl]-2-(4,6-dimethylpyrazolo[1,5-a]pyrazin-2-yl)-9-methyl-4H-pyrido[1,2-a]pyrimidin-4-one, 7-[(3S)-3,4-dimethylpiperazin-1-yl]-2-(4,6-dimethylpyrazolo[1,5-a]pyrazin-2-yl)-9-methyl-4H-pyrido[1,2-a]pyrimidin-4-one, 2-(4,6-dimethylpyrazolo[1,5-a]pyrazin-2-yl)-9-methyl-7-[(3R,5 S)-3,4,5-trimethylpiperazin-1-yl]-4H-pyrido[1,2-a]pyrimidin-4-one, 2-(2-methylimidazo[1,2-a]pyridin-6-yl)-7-[1-(propan-2-yl)-1,2,3,6-tetrahydropyridin-4-yl]-4H-pyrido[1,2-a]pyrimidin-4-one, 7-{4-[(dimethylamino)methyl]piperidin-1-yl}-2-(4,6-dimethylpyrazolo[1,5-a]pyrazin-2-yl)-4H-pyrido[1,2-a]pyrimidin-4-one, 2-(4,6-dimethylpyrazolo[1,5-a]pyrazin-2-yl)-7-[4-(pyrrolidin-1-ylmethyl) piperidin-1-yl]-4H-pyrido[1,2-a]pyrimidin-4-one, 2-(4,6-dimethylpyrazolo[1,5-a]pyrazin-2-yl)-7-[4-(piperidin-1-ylmethyl) piperidin-1-yl]-4H-pyrido[1,2-a]pyrimidin-4-one, 7-[1-(2-hydroxyethyl) piperidin-4-yl]-2-(2-methylimidazo[1,2-a]pyridin-6-yl)-4H-pyrido[1,2-a]pyrimidin-4-one, 7-[1-(2-hydroxyethyl)-1,2,3,6-tetrahydropyridin-4-yl]-2-(2-methylimidazo[1,2-a]pyridin-6-yl)-4H-pyrido[1,2-a]pyrimidin-4-one, 2-(2-methyl-2H-indazol-5-yl)-7-(1-methylpiperidin-4-yl)-4H-pyrido[1,2-a]pyrimidin-4-one, 2-(4,6-dimethylpyrazolo[1,5-a]pyrazin-2-yl)-7-{4-[(2-hydroxyethyl) (methyl)amino]piperidin-1-yl}-9-methyl-4H-pyrido[1,2-a]pyrimidin-4-one, 2-(4,6-dimethylpyrazolo[1,5-a]pyrazin-2-yl)-9-methyl-7-[4-(propylamino) piperidin-1-yl]-4H-pyrido[1,2-a]pyrimidin-4-one, 7-(4-amino-4-methylpiperidin-1-yl)-2-(4,6-dimethylpyrazolo[1,5-a]pyrazin-2-yl)-4H-pyrido[1,2-a]pyrimidin-4-one, 2-(4,6-dimethylpyrazolo[1,5-a]pyrazin-2-yl)-7-[4-(ethylamino) piperidin-1-yl]-9-methyl-4H-pyrido[1,2-a]pyrimidin-4-one, 7-{4-[bis(2-hydroxyethyl)amino]piperidin-1-yl}-2-(4,6-dimethylpyrazolo[1,5-a]pyrazin-2-yl)-9-methyl-4H-pyrido[1,2-a]pyrimidin-4-one, 2-(4,6-dimethylpyrazolo[1,5-a]pyrazin-2-yl)-7-[1-(oxetan-3-yl) piperidin-4-yl]-4H-pyrido[1,2-a]pyrimidin-4-one, 2-(5,7-dimethylfuro [2,3-c]pyridin-2-yl)-7-(4-ethylpiperazin-1-yl)-4H-pyrido[1,2-a]pyrimidin-4-one, 2-(4,6-dimethylpyrazolo[1,5-a]pyrazin-2-yl)-7-(1-methyloctahydro-5H-pyrrolo[3,2-c]pyridin-5-yl)-4H-pyrido[1,2-a]pyrimidin-4-one, 2-(1-methyl-1H-indazol-5-yl)-7-(piperidin-4-yl)-4H-pyrido[1,2-a]pyrimidin-4-one, 7-(1-ethylpiperidin-4-yl)-2-(1-methyl-1H-indazol-5-yl)-4H-pyrido[1,2-a]pyrimidin-4-one, 7-[1-(2-hydroxyethyl) piperidin-4-yl]-2-(1-methyl-1H-indazol-5-yl)-4H-pyrido[1,2-a]pyrimidin-4-one, 2-(2-methyl-2H-indazol-5-yl)-7-(piperidin-4-yl)-4H-pyrido[1,2-a]pyrimidin-4-one, 2-(2, 8-dimethylimidazo[1,2-a]pyridin-6-yl)-7-(piperidin-4-yl)-4H-pyrido[1,2-a]pyrimidin-4-one, 2-(2-methyl-2H-indazol-5-yl)-7-[1-(propan-2-yl) piperidin-4-yl]-4H-pyrido[1,2-a]pyrimidin-4-one, 2-(4,6-dimethylpyrazolo[1,5-a]pyrazin-2-yl)-7-{4-[(2-hydroxyethyl)amino]piperidin-1-yl}-9-methyl-4H-pyrido[1,2-a]pyrimidin-4-one, 2-(4,6-dimethylpyrazolo[1,5-a]pyrazin-2-yl)-9-methyl-7-[4-(methylamino) piperidin-1-yl]-4H-pyrido[1,2-a]pyrimidin-4-one, 2-(4,6-dimethylpyrazolo[1,5-a]pyrazin-2-yl)-9-methyl-7-[4-(propan-2-ylamino) piperidin-1-yl]-4H-pyrido[1,2-a]pyrimidin-4-one, 7-(1-ethylpiperidin-4-yl)-2-(2-methyl-2H-indazol-5-yl)-4H-pyrido[1,2-a]pyrimidin-4-one, 7-[1-(2-hydroxyethyl) piperidin-4-yl]-2-(2-methyl-2H-indazol-5-yl)-4H-pyrido[1,2-a]pyrimidin-4-one, 2-(2,8-dimethylimidazo[1,2-a]pyridin-6-yl)-7-(1-methylpiperidin-4-yl)-4H-pyrido[1,2-a]pyrimidin-4-one, 2-(2,8-dimethylimidazo[1,2-a]pyridin-6-yl)-7-(1-ethylpiperidin-4-yl)-4H-pyrido[1,2-a]pyrimidin-4-one, 2-(2,8-dimethylimidazo[1,2-a]pyridin-6-yl)-7-[1-(propan-2-yl) piperidin-4-yl]-4H-pyrido[1,2-a]pyrimidin-4-one, 2-(2,8-dimethylimidazo[1,2-a]pyridin-6-yl)-7-[1-(2-hydroxyethyl) piperidin-4-yl]-4H-pyrido[1,2-a]pyrimidin-4-one, 2-(4-ethyl-6-methylpyrazolo[1,5-a]pyrazin-2-yl)-7-(4-propylpiperazin-1-yl)-4H-pyrido[1,2-a]pyrimidin-4-one, 2-(4-ethyl-6-methylpyrazolo[1,5-a]pyrazin-2-yl)-7-[4-(propan-2-yl) piperazin-1-yl]-4H-pyrido[1,2-a]pyrimidin-4-one, 7-(4-cyclopropylpiperazin-1-yl)-2-(4-ethyl-6-methylpyrazolo[1,5-a]pyrazin-2-yl)-4H-pyrido[1,2-a]pyrimidin-4-one, 7-(4-cyclobutylpiperazin-1-yl)-2-(4-ethyl-6-methylpyrazolo[1,5-a]pyrazin-2-yl)-4H-pyrido[1,2-a]pyrimidin-4-one, 2-(4-ethyl-6-methylpyrazolo[1,5-a]pyrazin-2-yl)-7-[4-(oxetan-3-yl) piperazin-1-yl]-4H-pyrido[1,2-a]pyrimidin-4-one, 2-(4,6-dimethylpyrazolo[1,5-a]pyrazin-2-yl)-7-(1-ethyloctahydro-5H-pyrrol o[3,2-c]pyridin-5-yl)-4H-pyrido[1,2-a]pyrimidin-4-one, 2-(4,6-dimethylpyrazolo[1,5-a]pyrazin-2-yl)-7-[1-(2-hydroxyethyl)octahydro-5H-pyrrolo[3,2-c]pyridin-5-yl]-4H-pyrido[1,2-a]pyrimidin-4-one, 2-(4-methoxy-6-methylpyrazolo[1,5-a]pyrazin-2-yl)-7-(4-methylpiperazin-1-yl)-4H-pyrido[1,2-a]pyrimidin-4-one, 2-(4-hydroxy-6-methylpyrazolo[1,5-a]pyrazin-2-yl)-7-(4-methylpiperazin-1-yl)-4H-pyrido[1,2-a]pyrimidin-4-one, 7-[(3R)-3,4-dimethylpiperazin-1-yl]-2-(4-ethyl-6-methylpyrazolo[1,5-a]pyrazin-2-yl)-4H-pyrido[1,2-a]pyrimidin-4-one, 7-[(3R)-4-ethyl-3-methylpiperazin-1-yl]-2-(4-ethyl-6-methylpyrazolo[1,5-a]pyrazin-2-yl)-4H-pyrido[1,2-a]pyrimidin-4-one, 2-(4-ethyl-6-methylpyrazolo[1,5-a]pyrazin-2-yl)-7-[(3R)-3-methyl-4-propylpiperazin-1-yl]-4H-pyrido[1,2-a]pyrimidin-4-one, 2-(4-ethyl-6-methylpyrazolo[1,5-a]pyrazin-2-yl)-7-[(3R)-4-(2-hydroxyethyl)-3-methylpiperazin-1-yl]-4H-pyrido[1,2-a]pyrimidin-4-one, 7-[(3R,5 S)-3,5-dimethylpiperazin-1-yl]-2-(4-ethyl-6-methylpyrazolo[1,5-a]pyrazin-2-yl)-4H-pyrido[1,2-a]pyrimidin-4-one, 2-(4-ethyl-6-methylpyrazolo[1,5-a]pyrazin-2-yl)-7-[4-(pyrrolidin-1-yl) piperidin-1-yl]-4H-pyrido[1,2-a]pyrimidin-4-one, 2-(4-ethyl-6-methylpyrazolo[1,5-a]pyrazin-2-yl)-7-[(3 S)-3-methylpiperazin-1-yl]-4H-pyrido[1,2-a]pyrimidin-4-one, 2-(4-ethyl-6-methylpyrazolo[1,5-a]pyrazin-2-yl)-7-(4-methyl-1,4-diazepan-1-yl)-4H-pyrido[1,2-a]pyrimidin-4-one, 2-(8-ethyl-2-methylimidazo[1,2-a]pyridin-6-yl)-7-(piperazin-1-yl)-4H-pyrido[1,2-a]pyrimidin-4-one, 2-(8-ethyl-2-methylimidazo[1,2-a]pyridin-6-yl)-7-[(3R)-3-methylpiperazin-1-yl]-4H-pyrido[1,2-a]pyrimidin-4-one, 2-(8-ethyl-2-methylimidazo[1,2-a]pyridin-6-yl)-7-[(3S)-3-methylpiperazin-1-yl]-4H-pyrido[1,2-a]pyrimidin-4-one, 2-(4,6-dimethylpyrazolo[1,5-a]pyrazin-2-yl)-7-[1-(propan-2-yl) piperidin-4-yl]-4H-pyrido[1,2-a]pyrimidin-4-one, 7-(1-cyclopropylpiperidin-4-yl)-2-(4,6-dimethylpyrazolo[1,5-a]pyrazin-2-yl)-4H-pyrido[1,2-a]pyrimidin-4-one, 7-(1-cyclobutylpiperidin-4-yl)-2-(4,6-dimethylpyrazolo[1,5-a]pyrazin-2-yl)-4H-pyrido[1,2-a]pyrimidin-4-one, 2-(4-ethyl-6-methylpyrazolo[1,5-a]pyrazin-2-yl)-7-[(3R)-3-methyl-4-(propan-2-yl) piperazin-1-yl]-4H-pyrido[1,2-a]pyrimidin-4-one, 7-[(3R)-4-cyclopropyl-3-methylpiperazin-1-yl]-2-(4-ethyl-6-methylpyrazolo[1,5-a]pyrazin-2-yl)-4H-pyrido[1,2-a]pyrimidin-4-one, 7-[(3R)-4-cyclobutyl-3-methylpiperazin-1-yl]-2-(4-ethyl-6-methylpyrazolo[1,5-a]pyrazin-2-yl)-4H-pyrido[1,2-a]pyrimidin-4-one, 2-(4-ethyl-6-methylpyrazolo[1,5-a]pyrazin-2-yl)-7-[(3R)-3-methyl-4-(oxetan-3-yl) piperazin-1-yl]-4H-pyrido[1,2-a]pyrimidin-4-one, 2-(8-ethyl-2-methylimidazo[1,2-a]pyridin-6-yl)-7-[4-(2-hydroxyethyl) piperazin-1-yl]-4H-pyrido[1,2-a]pyrimidin-4-one, 7-(4-cyclobutylpiperazin-1-yl)-2-(8-ethyl-2-methylimidazo[1,2-a]pyridin-6-yl)-4H-pyrido[1,2-a]pyrimidin-4-one, 2-(8-ethyl-2-methylimidazo[1,2-a]pyridin-6-yl)-7-[(3R)-4-(2-hydroxyethyl)-3-methylpiperazin-1-yl]-4H-pyrido[1,2-a]pyrimidin-4-one, 7-[(3R)-4-cyclobutyl-3-methylpiperazin-1-yl]-2-(8-ethyl-2-methylimidazo[1,2-a]pyridin-6-yl)-4H-pyrido[1,2-a]pyrimidin-4-one, 2-(8-ethyl-2-methylimidazo[1,2-a]pyridin-6-yl)-7-[(3S)-4-(2-hydroxyethyl)-3-methylpiperazin-1-yl]-4H-pyrido[1,2-a]pyrimidin-4-one, 7-[(3S)-4-cyclobutyl-3-methylpiperazin-1-yl]-2-(8-ethyl-2-methylimidazo[1,2-a]pyridin-6-yl)-4H-pyrido[1,2-a]pyrimidin-4-one, 2-(4-ethyl-6-methylpyrazolo[1,5-a]pyrazin-2-yl)-7-(piperidin-4-yl)-4H-pyrido[1,2-a]pyrimidin-4-one, 2-(4-ethyl-6-methylpyrazolo[1,5-a]pyrazin-2-yl)-7-(1-methylpiperidin-4-yl)-4H-pyrido[1,2-a]pyrimidin-4-one, 2-(4-ethyl-6-methylpyrazolo[1,5-a]pyrazin-2-yl)-7-(1-ethylpiperidin-4-yl)-4H-pyrido[1,2-a]pyrimidin-4-one, 2-(4-ethyl-6-methylpyrazolo[1,5-a]pyrazin-2-yl)-7-[1-(2-hydroxyethyl) piperidin-4-yl]-4H-pyrido[1,2-a]pyrimidin-4-one, 2-(4-ethyl-6-methylpyrazolo[1,5-a]pyrazin-2-yl)-7-(1-propylpiperidin-4-yl)-4H-pyrido[1,2-a]pyrimidin-4-one, 2-(4-ethyl-6-methylpyrazolo[1,5-a]pyrazin-2-yl)-7-[(3R)-3-methylpiperazin-1-yl]-4H-pyrido[1,2-a]pyrimidin-4-one, 2-(4,6-dimethylpyrazolo[1,5-a]pyrazin-2-yl)-7-[1-(2-fluoroethyl) piperidin-4-yl]-4H-pyrido[1,2-a]pyrimidin-4-one, 2-(4,6-dimethylpyrazolo[1,5-a]pyrazin-2-yl)-7-[1-(3-fluoropropyl) piperidin-4-yl]-4H-pyrido[1,2-a]pyrimidin-4-one, 2-(4-ethyl-6-methylpyrazolo[1,5-a]pyrazin-2-yl)-7-[4-(2-fluoroethyl) piperazin-1-yl]-4H-pyrido[1,2-a]pyrimidin-4-one, 2-(4-ethyl-6-methylpyrazolo[1,5-a]pyrazin-2-yl)-7-[4-(3-fluoropropyl) piperazin-1-yl]-4H-pyrido[1,2-a]pyrimidin-4-one, 2-(4-ethyl-6-methylpyrazolo[1,5-a]pyrazin-2-yl)-7-[(3R)-4-(2-fluoroethyl)-3-methylpiperazin-1-yl]-4H-pyrido[1,2-a]pyrimidin-4-one, 2-(4-ethyl-6-methylpyrazolo[1,5-a]pyrazin-2-yl)-7-[(3R)-4-(3-fluoropropyl)-3-methylpiperazin-1-yl]-4H-pyrido[1,2-a]pyrimidin-4-one, 2-(4-ethyl-6-methylpyrazolo[1,5-a]pyrazin-2-yl)-7-[1-(2-fluoroethyl) piperidin-4-yl]-4H-pyrido[1,2-a]pyrimidin-4-one, 2-(4-ethyl-6-methylpyrazolo[1,5-a]pyrazin-2-yl)-7-[1-(3-fluoropropyl) piperidin-4-yl]-4H-pyrido[1,2-a]pyrimidin-4-one, 2-(4,6-dimethylpyrazolo[1,5-a]pyrazin-2-yl)-7- {(3R)-4-[2-(2-hydroxyethoxy)ethyl]-3-methylpiperazin-1-yl}-9-methyl-4H-pyrido[1,2-a]pyrimidin-4-one, 2-(4,6-dimethylpyrazolo[1,5-a]pyrazin-2-yl)-9-methyl-7-(piperidin-4-yl)-4H-pyrido[1,2-a]pyrimidin-4-one, 2-(4,6-dimethylpyrazolo[1,5-a]pyrazin-2-yl)-9-methyl-7-(1-methylpiperidin-4-yl)-4H-pyrido[1,2-a]pyrimidin-4-one, 2-(4,6-dimethylpyrazolo[1,5-a]pyrazin-2-yl)-7-[(3R)-4-(2-hydroxyethyl)-3-methylpiperazin-1-yl]-9-methyl-4H-pyrido[1,2-a]pyrimidin-4-one, 2-[8-(hydroxymethyl)-2-methylimidazo[1,2-a]pyridin-6-yl]-7-(piperazin-1-yl)-4H-pyrido[1,2-a]pyrimidin-4-one, 7-[(3R,5S)-3,5-dimethylpiperazin-1-yl]-2-(8-ethyl-2-methylimidazo[1,2-a]pyridin-6-yl)-4H-pyrido[1,2-a]pyrimidin-4-one, 2-(8-ethyl-2-methylimidazo[1,2-a]pyridin-6-yl)-7-(4-methyl-1,4-diazepan-1-yl)-4H-pyrido[1,2-a]pyrimidin-4-one, 2-[8-(hydroxymethyl)-2-methylimidazo[1,2-a]pyridin-6-yl]-7-(4-methylpiperazin-1-yl)-4H-pyrido[1,2-a]pyrimidin-4-one, 7-(4-ethylpiperazin-1-yl)-2-[8-(hydroxymethyl)-2-methylimidazo[1,2-a]pyridin-6-yl]-4H-pyrido[1,2-a]pyrimidin-4-one, 2-(4-ethyl-6-methylpyrazolo[1,5-a]pyrazin-2-yl)-7-[1-(propan-2-yl) piperidin-4-yl]-4H-pyrido[1,2-a]pyrimidin-4-one, 7-(1-cyclopropylpiperidin-4-yl)-2-(4-ethyl-6-methylpyrazolo[1,5-a]pyrazin-2-yl)-4H-pyrido[1,2-a]pyrimidin-4-one, 7-(1-cyclobutylpiperidin-4-yl)-2-(4-ethyl-6-methylpyrazolo[1,5-a]pyrazin-2-yl)-4H-pyrido[1,2-a]pyrimidin-4-one, 2-(4-ethyl-6-methylpyrazolo[1,5-a]pyrazin-2-yl)-7-[1-(oxetan-3-yl) piperidin-4-yl]-4H-pyrido[1,2-a]pyrimidin-4-one, 2-(4-cyclopropyl-6-methylpyrazolo[1,5-a]pyrazin-2-yl)-7-(piperazin-1-yl)-4H-pyrido[1,2-a]pyrimidin-4-one, 2-(4-cyclopropyl-6-methylpyrazolo[1,5-a]pyrazin-2-yl)-7-(4-methylpiperazin-1-yl)-4H-pyrido[1,2-a]pyrimidin-4-one, 2-(4-cyclopropyl-6-methylpyrazolo[1,5-a]pyrazin-2-yl)-7-(4-ethylpiperazin-1-yl)-4H-pyrido[1,2-a]pyrimidin-4-one, 2-(4-cyclopropyl-6-methylpyrazolo[1,5-a]pyrazin-2-yl)-7-[4-(2-hydroxyethyl) piperazin-1-yl]-4H-pyrido[1,2-a]pyrimidin-4-one, 2-(4,6-dimethylpyrazolo[1,5-a]pyrazin-2-yl)-7-(1-propylpiperidin-4-yl)-4H-pyrido[1,2-a]pyrimidin-4-one, 2-[4-(dimethylamino)-6-methylpyrazolo[1,5-a]pyrazin-2-yl]-7-(piperazin-1-yl)-4H-pyrido[1,2-a]pyrimidin-4-one, 2-(2-methyl-1H-benzimidazol-6-yl)-7-(4-methylpiperazin-1-yl)-4H-pyrido[1,2-a]pyrimidin-4-one, 7-(4-ethylpiperazin-1-yl)-2-(2-methyl-1H-benzimidazol-6-yl)-4H-pyrido[1,2-a]pyrimidin-4-one, 7-[1-(2,2-dimethyl-1,3-dioxan-5-yl) piperidin-4-yl]-2-(4-ethyl-6-methylpyrazolo[1,5-a]pyrazin-2-yl)-4H-pyrido[1,2-a]pyrimidin-4-one, 7-[1-(1,3-dihydroxypropan-2-yl) piperidin-4-yl]-2-(4-ethyl-6-methylpyrazolo[1,5-a]pyrazin-2-yl)-4H-pyrido[1,2-a]pyrimidin-4-one, 7-[(3R,5S)-3,5-dimethylpiperazin-1-yl]-2-(1,3-dimethylpyrrolo[1,2-a]pyrazin-7-yl)-4H-pyrido[1,2-a]pyrimidin-4-one, 2-(8-ethyl-2-methylimidazo[1,2-a]pyridin-6-yl)-7-(piperidin-4-yl)-4H-pyrido[1,2-a]pyrimidin-4-one, 2-(8-ethyl-2-methylimidazo[1,2-a]pyridin-6-yl)-7-(1-methylpiperidin-4-yl)-4H-pyrido[1,2-a]pyrimidin-4-one, 2-(8-ethyl-2-methylimidazo[1,2-a]pyridin-6-yl)-7-(1-ethylpiperidin-4-yl)-4H-pyrido[1,2-a]pyrimidin-4-one, 2-(8-ethyl-2-methylimidazo[1,2-a]pyridin-6-yl)-7-[1-(2-hydroxyethyl) piperidin-4-yl]-4H-pyrido[1,2-a]pyrimidin-4-one, 7-[(3R)-3,4-dimethylpiperazin-1-yl]-2-(8-ethyl-2-methylimidazo[1,2-a]pyridin-6-yl)-4H-pyrido[1,2-a]pyrimidin-4-one, 2-(8-ethyl-2-methylimidazo[1,2-a]pyridin-6-yl)-7-[(3R)-4-ethyl-3-methylpiperazin-1-yl]-4H-pyrido[1,2-a]pyrimidin-4-one, 2-(4,6-dimethylpyrazolo[1,5-a]pyrazin-2-yl)-7-(1-ethylpiperidin-4-yl)-9-methyl-4H-pyrido[1,2-a]pyrimidin-4-one, 2-(4,6-dimethylpyrazolo[1,5-a]pyrazin-2-yl)-7-[1-(2-hydroxyethyl) piperidin-4-yl]-9-methyl-4H-pyrido[1,2-a]pyrimidin-4-one, 7-(1-cyclobutylpiperidin-4-yl)-2-(4,6-dimethylpyrazolo[1,5-a]pyrazin-2-yl)-9-methyl-4H-pyrido[1,2-a]pyrimidin-4-one, 9-methyl-2-(2-methyl-2H-indazol-5-yl)-7-(1-methylpiperidin-4-yl)-4H-pyrido[1,2-a]pyrimidin-4-one, 7-[4-(dimethylamino)-4-methylpiperidin-1-yl]-2-(4,6-dimethylpyrazolo[1,5-a]pyrazin-2-yl)-4H-pyrido[1,2-a]pyrimidin-4-one, 2-(4,6-dimethylpyrazolo[1,5-a]pyrazin-2-yl)-7-[4-(ethylamino)-4-methylpiperidin-1-yl]-4H-pyrido[1,2-a]pyrimidin-4-one, 2-(4,6-dimethylpyrazolo[1,5-a]pyrazin-2-yl)-7-[4-methyl-4-(propylamino) piperidin-1-yl]-4H-pyrido[1,2-a]pyrimidin-4-one, 2-(4,6-dimethylpyrazolo[1,5-a]pyrazin-2-yl)-7-{4-[(2-hydroxyethyl)amino]-4-methylpiperidin-1-yl}-4H-pyrido[1,2-a]pyrimidin-4-one, 7-(1-cyclobutylpiperidin-4-yl)-9-methyl-2-(2-methyl-2H-indazol-5-yl)-4H-pyrido[1,2-a]pyrimidin-4-one, 7-[1-(2-hydroxyethyl) piperidin-4-yl]-9-methyl-2-(2-methyl-2H-indazol-5-yl)-4H-pyrido[1,2-a]pyrimidin-4-one, 2-(4,6-dimethylpyrazolo[1,5-a]pyrazin-2-yl)-9-methyl-7-(1-propylpiperidin-4-yl)-4H-pyrido[1,2-a]pyrimidin-4-one, 2-(1,3-dimethylpyrrolo[1,2-a]pyrazin-7-yl)-7-[(3R)-3-methylpiperazin-1-yl]-4H-pyrido[1,2-a]pyrimidin-4-one, 2-(8-cyclopropyl-2-methylimidazo[1,2-a]pyridin-6-yl)-7-(piperazin-1-yl)-4H-pyrido[1,2-a]pyrimidin-4-one, 2-(8-cyclopropyl-2-methylimidazo[1,2-a]pyridin-6-yl)-7-[(3R)-3-methylpiperazin-1-yl]-4H-pyrido[1,2-a]pyrimidin-4-one, 2-(8-cyclopropyl-2-methylimidazo[1,2-a]pyridin-6-yl)-7-[(3 S)-3-methylpiperazin-1-yl]-4H-pyrido[1,2-a]pyrimidin-4-one, 2-(8-cyclopropyl-2-methylimidazo[1,2-a]pyridin-6-yl)-7-[(3R,5S)-3, 5-dimethylpiperazin-1-yl]-4H-pyrido[1,2-a]pyrimidin-4-one, 7-(1-cyclopropylpiperidin-4-yl)-9-methyl-2-(2-methyl-2H-indazol-5-yl)-4H-pyrido[1,2-a]pyrimidin-4-one, 7-(1-ethylpiperidin-4-yl)-9-methyl-2-(2-methyl-2H-indazol-5-yl)-4H-pyrido[1,2-a]pyrimidin-4-one, 2-(4,6-dimethylpyrazolo[1,5-a]pyrazin-2-yl)-9-methyl-7-(piperazin-1-yl)-4H-pyrido[1,2-a]pyrimidin-4-one, 9-methyl-2-(2-methyl-2H-indazol-5-yl)-7-(piperidin-4-yl)-4H-pyrido[1,2-a]pyrimidin-4-one, 2-(4,6-dimethylpyrazolo[1,5-a]pyrazin-2-yl)-7-[(1-methylpiperidin-4-yl)oxy]-4H-pyrido[1,2-a]pyrimidin-4-one, 2-(6-methyl-4-propylpyrazolo[1,5-a]pyrazin-2-yl)-7-(piperazin-1-yl)-4H-pyrido[1,2-a]pyrimidin-4-one, 7-(4-methylpiperazin-1-yl)-2-(6-methyl-4-propylpyrazolo[1,5-a]pyrazin-2-yl)-4H-pyrido[1,2-a]pyrimidin-4-one, 7-(4-ethylpiperazin-1-yl)-2-(6-methyl-4-propylpyrazolo[1,5-a]pyrazin-2-yl)-4H-pyrido[1,2-a]pyrimidin-4-one, 7-[4-(2-hydroxyethyl) piperazin-1-yl]-2-(6-methyl-4-propylpyrazolo[1,5-a]pyrazin-2-yl)-4H-pyrido[1,2-a]pyrimidin-4-one, 7-[(3R)-3-methylpiperazin-1-yl]-2-(6-methyl-4-propylpyrazolo[1,5-a]pyrazin-2-yl)-4H-pyrido[1,2-a]pyrimidin-4-one, 7-[(3S)-3-methylpiperazin-1-yl]-2-(6-methyl-4-propylpyrazolo[1,5-a]pyrazin-2-yl)-4H-pyrido[1,2-a]pyrimidin-4-one, 7-[(3R,5S)-3,5-dimethylpiperazin-1-yl]-2-(6-methyl-4-propylpyrazolo[1,5-a]pyrazin-2-yl)-4H-pyrido[1,2-a]pyrimidin-4-one, 2-(1,3-dimethylpyrrolo[1,2-a]pyrazin-7-yl)-7-[(3R)-3-methyl-4-(propan-2-yl) piperazin-1-yl]-4H-pyrido[1,2-a]pyrimidin-4-one, 7-(4-amino-4-methylpiperidin-1-yl)-2-(1,3-dimethylpyrrolo[1,2-a]pyrazin-7-yl)-4H-pyrido[1,2-a]pyrimidin-4-one, 2-(4,6-dimethylpyrazolo[1,5-a]pyrazin-2-yl)-7-[(3 S)-3-ethylpiperazin-1-yl]-9-methyl-4H-pyrido[1,2-a]pyrimidin-4-one, 2-[2-methyl-8-(trifluoromethyl) imidazo[1,2-a]pyridin-6-yl]-7-(piperazin-1-yl)-4H-pyrido[1,2-a]pyrimidin-4-one, 7-[(3R)-3-methylpiperazin-1-yl]-2-[2-methyl-8-(trifluoromethyl) imidazo[1,2-a]pyridin-6-yl]-4H-pyrido[1,2-a]pyrimidin-4-one, 7-[(3S)-3-methylpiperazin-1-yl]-2-[2-methyl-8-(trifluoromethyl) imidazo[1,2-a]pyridin-6-yl]-4H-pyrido[1,2-a]pyrimidin-4-one, 7-[(3R,5 S)-3,5-dimethylpiperazin-1-yl]-2-[2-methyl-8-(trifluoromethyl) imidazo[1,2-a]pyridin-6-yl]-4H-pyrido[1,2-a]pyrimidin-4-one, 7-(4-amino-4-methylpiperidin-1-yl)-2-(4-ethyl-6-methylpyrazolo[1,5-a]pyrazin-2-yl)-4H-pyrido[1,2-a]pyrimidin-4-one, 2-(2,7-dimethyl-2H-indazol-5-yl)-7-(piperazin-1-yl)-4H-pyrido[1,2-a]pyrimidin-4-one, 2-(2,7-dimethyl-2H-indazol-5-yl)-7-(piperidin-4-yl)-4H-pyrido[1,2-a]pyrimidin-4-one, 2-(2,7-dimethyl-2H-indazol-5-yl)-7-[(3R)-3-methylpiperazin-1-yl]-4H-pyrido[1,2-a]pyrimidin-4-one, 7-(3-aminopropyl)-2-(4,6-dimethylpyrazolo[1,5-a]pyrazin-2-yl)-4H-pyrido[1,2-a]pyrimidin-4-one, 2-(1,3-dimethylpyrrolo[1,2-a]pyrazin-7-yl)-7-(1,2,3,6-tetrahydropyridin-4-yl)-4H-pyrido[1,2-a]pyrimidin-4-one, 2-(4,6-dimethylpyrazolo[1,5-a]pyrazin-2-yl)-7-(2,2,6,6-tetramethyl-1,2,3,6-tetrahydropyridin-4-yl)-4H-pyrido[1,2-a]pyrimidin-4-one, 2-(4-ethyl-6-methylpyrazolo[1,5-a]pyrazin-2-yl)-7-(2,2,6,6-tetramethyl-1,2,3,6-tetrahydropyridin-4-yl)-4H-pyrido[1,2-a]pyrimidin-4-one, 2-(4,6-dimethylpyrazolo[1,5-a]pyrazin-2-yl)-7-[(3 aR,6aS)-hexahydropyrrolo[3,4-c]pyrrol-2 (1H)-yl]-4H-pyrido[1,2-a]pyrimidin-4-one, 2-(4-ethyl-6-methylpyrazolo[1,5-a]pyrazin-2-yl)-7-[(3 aR,6aS)-hexahydropyrrolo[3,4-c]pyrrol-2 (1H)-yl]-4H-pyrido[1,2-a]pyrimidin-4-one, 7-[(3R,5S)-3,5-dimethylpiperazin-1-yl]-2-(1-ethyl-3-methylpyrrolo[1,2-a]pyrazin-7-yl)-4H-pyrido[1,2-a]pyrimidin-4-one, 7-(1,4-diazepan-1-yl)-2-(1-ethyl-3-methylpyrrolo[1,2-a]pyrazin-7-yl)-4H-pyrido[1,2-a]pyrimidin-4-one, 2-(2,7-dimethyl-2H-indazol-5-yl)-7 [(3S)-3-methylpiperazin-1-yl]-4H-pyrido[1,2-a]pyrimidin-4-one, 2-(2,7-dimethyl-2H-indazol-5-yl)-7-[(3 S)-3,4-dimethylpiperazin-1-yl]-4H-pyrido[1,2-a]pyrimidin-4-one, 2-(2,7-dimethyl-2H-indazol-5-yl)-7-(1-methylpiperidin-4-yl)-4H-pyrido[1,2-a]pyrimidin-4-one, 2-(2,7-dimethyl-2H-indazol-5-yl)-7-(1-ethylpiperidin-4-yl)-4H-pyrido[1,2-a]pyrimidin-4-one, 9-methyl-2-(2-methyl-2H-indazol-5-yl)-7-(piperazin-1-yl)-4H-pyrido[1,2-a]pyrimidin-4-one, 9-methyl-2-(2-methyl-2H-indazol-5-yl)-7-[(3R)-3-methylpiperazin-1-yl]-4H-pyrido[1,2-a]pyrimidin-4-one, 9-methyl-2-(2-methyl-2H-indazol-5-yl)-7-[(3 S)-3-methylpiperazin-1-yl]-4H-pyrido[1,2-a]pyrimidin-4-one, 7-[3-(dimethylamino) azetidin-1-yl]-2-(4,6-dimethylpyrazolo[1,5-a]pyrazin-2-yl)-4H-pyrido[1,2-a]pyrimidin-4-one, 7-[3-(diethylamino) azetidin-1-yl]-2-(4,6-dimethylpyrazolo[1,5-a]pyrazin-2-yl)-4H-pyrido[1,2-a]pyrimidin-4-one, 2-(4,6-dimethylpyrazolo[1,5-a]pyrazin-2-yl)-7-[3-(pyrrolidin-1-yl) azetidin-1-yl]-4H-pyrido[1,2-a]pyrimidin-4-one, 7-(1,4-diazepan-1-yl)-2-(4-ethyl-6-methylpyrazolo[1,5-a]pyrazin-2-yl)-4H-pyrido[1,2-a]pyrimidin-4-one, 7-[(3aR,6aS)-hexahydropyrrolo[3,4-c]pyrrol-2 (1H)-yl]-2-(6-methyl-4-propylpyrazolo[1,5-a]pyrazin-2-yl)-4H-pyrido[1,2-a]pyrimidin-4-one, 2-(6-methyl-4-propylpyrazolo[1,5-a]pyrazin-2-yl)-7-(1,2,3,6-tetrahydropyridin-4-yl)-4H-pyrido[1,2-a]pyrimidin-4-one, 2-(4-ethyl-6-methylpyrazolo[1,5-a]pyrazin-2-yl)-7-(1,2,3,6-tetrahydropyridin-4-yl)-4H-pyrido[1,2-a]pyrimidin-4-one, 7-[(3 S)-3-(aminomethyl) pyrrolidin-1-yl]-2-(4,6-dimethylpyrazolo[1,5-a]pyrazin-2-yl)-4H-pyrido[1,2-a]pyrimidin-4-one, 2-(4,6-dimethylpyrazolo[1,5-a]pyrazin-2-yl)-7-[3-(piperidin-1-yl) azetidin-1-yl]-4H-pyrido[1,2-a]pyrimidin-4-one, 2-(6-methyl-4-propylpyrazolo[1,5-a]pyrazin-2-yl)-7-(piperidin-4-yl)-4H-pyrido[1,2-a]pyrimidin-4-one, 7-(2,7-diazaspiro[4.4]non-2-yl)-2-(4,6-dimethylpyrazolo[1,5-a]pyrazin-2-yl)-4H-pyrido[1,2-a]pyrimidin-4-one, 2-(2,7-dimethyl-2H-indazol-5-yl)-7-(4-methylpiperazin-1-yl)-4H-pyrido[1,2-a]pyrimidin-4-one, 7- {(3S)-3-[(dimethylamino)methyl]pyrrolidin-1-yl}-2-(4,6-dimethylpyrazolo[1,5-a]pyrazin-2-yl)-4H-pyrido[1,2-a]pyrimidin-4-one, 2-(4-ethyl-6-methylpyrazolo[1,5-a]pyrazin-2-yl)-9-methyl-7-(piperidin-4-yl)-4H-pyrido[1,2-a]pyrimidin-4-one, 9-methyl-2-(1-methyl-1H-indazol-5-yl)-7-(piperidin-4-yl)-4H-pyrido[1,2-a]pyrimidin-4-one, 2-(4-ethyl-6-methylpyrazolo[1,5-a]pyrazin-2-yl)-9-methyl-7-(1,2,3,6-tetrahydropyridin-4-yl)-4H-pyrido[1,2-a]pyrimidin-4-one, 2-(4-ethyl-6-methylpyrazolo[1,5-a]pyrazin-2-yl)-9-methyl-7-(1-methylpiperidin-4-yl)-4H-pyrido[1,2-a]pyrimidin-4-one, 2-(1,7-dimethyl-1H-indazol-5-yl)-7-(piperidin-4-yl)-4H-pyrido[1,2-a]pyrimidin-4-one, 2-(1,7-dimethyl-1H-indazol-5-yl)-7-(piperazin-1-yl)-4H-pyrido[1,2-a]pyrimidin-4-one, 2-(1,7-dimethyl-1H-indazol-5-yl)-7-[(3 S)-3-methylpiperazin-1-yl]-4H-pyrido[1,2-a]pyrimidin-4-one, 7- {(3S)-3-[(diethylamino)methyl]pyrrolidin-1-yl}-2-(4,6-dimethylpyrazolo[1,5-a]pyrazin-2-yl)-4H-pyrido[1,2-a]pyrimidin-4-one, 2-(4,6-dimethylpyrazolo[1,5-a]pyrazin-2-yl)-7- {(3S)-3-[(ethylamino)methyl]pyrrolidin-1-yl}-4H-pyrido[1,2-a]pyrimidin-4-one, 7-{3-[(dimethylamino)methyl]azetidin-1-yl}-2-(4,6-dimethylpyrazolo[1,5-a]pyrazin-2-yl)-4H-pyrido[1,2-a]pyrimidin-4-one, 7-{3-[(diethylamino)methyl]azetidin-1-yl}-2-(4,6-dimethylpyrazolo[1,5-a]pyrazin-2-yl)-4H-pyrido[1,2-a]pyrimidin-4-one, 2-(1-ethyl-3-methylpyrrolo[1,2-a]pyrazin-7-yl)-7-[(8aS)-hexahydropyrrolo[1,2-a]pyrazin-2 (1H)-yl]-4H-pyrido[1,2-a]pyrimidin-4-one, 2-(4-ethyl-6-methylpyrazolo[1,5-a]pyrazin-2-yl)-9-methyl-7-[(3R)-3-methylpiperazin-1-yl]-4H-pyrido[1,2-a]pyrimidin-4-one, 9-methyl-2-(1-methyl-1H-indazol-5-yl)-7-(1-methylpiperidin-4-yl)-4H-pyrido[1,2-a]pyrimidin-4-one, 7-[(3R)-3,4-dimethylpiperazin-1-yl]-2-(4-ethyl-6-methylpyrazolo[1,5-a]pyrazin-2-yl)-9-methyl-4H-pyrido[1,2-a]pyrimidin-4-one, 7-(1-ethylpiperidin-4-yl)-9-methyl-2-(1-methyl-1H-indazol-5-yl)-4H-pyrido[1,2-a]pyrimidin-4-one, 2-(4-ethyl-6-methylpyrazolo[1,5-a]pyrazin-2-yl)-9-methyl-7-[(3 S)-3-methylpiperazin-1-yl]-4H-pyrido[1,2-a]pyrimidin-4-one, 7-[1-(2-hydroxyethyl) piperidin-4-yl]-9-methyl-2-(1-methyl-1H-indazol-5-yl)-4H-pyrido[1,2-a]pyrimidin-4-one, 7-[(3S)-3,4-dimethylpiperazin-1-yl]-2-(4-ethyl-6-methylpyrazolo[1,5-a]pyrazin-2-yl)-9-methyl-4H-pyrido[1,2-a]pyrimidin-4-one, 2-(4-ethyl-6-methylpyrazolo[1,5-a]pyrazin-2-yl)-7-(1-ethylpiperidin-4-yl)-9-methyl-4H-pyrido[1,2-a]pyrimidin-4-one, 7-(1-cyclobutylpiperidin-4-yl)-2-(4-ethyl-6-methylpyrazolo[1,5-a]pyrazin-2-yl)-9-methyl-4H-pyrido[1,2-a]pyrimidin-4-one, 2-(4-ethyl-6-methylpyrazolo[1,5-a]pyrazin-2-yl)-7-[1-(2-hydroxyethyl) piperidin-4-yl]-9-methyl-4H-pyrido[1,2-a]pyrimidin-4-one, 2-(8-ethyl-2-methylimidazo[1,2-a]pyridin-6-yl)-7-[(8aR)-hexahydropyrrolo[1,2-a]pyrazin-2 (1H)-yl]-4H-pyrido[1,2-a]pyrimidin-4-one, 2-(8-ethyl-2-methylimidazo[1,2-a]pyridin-6-yl)-7-[(8aS)-hexahydropyrrolo[1,2-a]pyrazin-2 (1H)-yl]-4H-pyrido[1,2-a]pyrimidin-4-one, 7-[(3R)-3-(aminomethyl) pyrrolidin-1-yl]-2-(4,6-dimethylpyrazolo[1,5-a]pyrazin-2-yl)-4H-pyrido[1,2-a]pyrimidin-4-one, 2-(4,6-dimethylpyrazolo[1,5-a]pyrazin-2-yl)-7-[(2S,6S)-2,6-dimethyl-1,2,3,6-tetrahydropyridin-4-yl]-4H-pyrido[1,2-a]pyrimidin-4-one, 7- {(3R)-3-[(dimethylamino)methyl]pyrrolidin-1-yl}-2-(4,6-dimethylpyrazolo[1,5-a]pyrazin-2-yl)-4H-pyrido[1,2-a]pyrimidin-4-one, 7-[(2S,6S)-2,6-dimethylpiperidin-4-yl]-2-(4,6-dimethylpyrazolo[1,5-a]pyrazin-2-yl)-4H-pyrido[1,2-a]pyrimidin-4-one, 2-(8-fluoro-2-methylimidazo[1,2-a]pyridin-6-yl)-7-[4-(2-hydroxyethyl) piperazin-1-yl]-4H-pyrido[1,2-a]pyrimidin-4-one, 2-(imidazo[1,2-a]pyridin-6-yl)-7-(4-methylpiperazin-1-yl)-4H-pyrido[1,2-a]pyrimidin-4-one, 2-(4-fluoro-2-methyl-1,3-benzoxazol-6-yl)-7-[(8aS)-hexahydropyrrolo[1,2-a]pyrazin-2 (1H)-yl]-4H-pyrido[1,2-a]pyrimidin-4-one, 7-(2,7-diazaspiro[3 0.5]non-7-yl)-2-(4,6-dimethylpyrazolo[1,5-a]pyrazin-2-yl)-4H-pyrido[1,2-a]pyrimidin-4-one, 7-(4-methylpiperazin-1-yl)-2-(2-methyl[1,2,4]triazolo[1,5-a]pyridin-6-yl)-4H-pyrido[1,2-a]pyrimidin-4-one, 7-(4-methylpiperazin-1-yl)-2-[2-methyl-8-(trifluoromethyl) imidazo[1,2-a]pyridin-6-yl]-4H-pyrido[1,2-a]pyrimidin-4-one, 2-methyl-6-[7-(4-methylpiperazin-1-yl)-4-oxo-4H-pyrido[1,2-a]pyrimidin-2-yl]imidazo[1,2-a]pyridine-8-carbonitrile 2-(2, 8-dimethylimidazo[1,2-a]pyridin-6-yl)-7-(4-methylpiperazin-1-yl)-4H-pyrido[1,2-a]pyrimidin-4-one, 7-(4,7-diazaspiro[2.5]oct-7-yl)-2-(2,8-dimethylimidazo[1,2-a]pyridin-6-yl)-4H-pyrido[1,2-a]pyrimidin-4-one, 2-(8-fluoro-2-methylimidazo[1,2-a]pyridin-6-yl)-7-(1-methylpiperidin-4-yl)-4H-pyrido[1,2-a]pyrimidin-4-one, 2-(8-fluoro-2-methylimidazo[1,2-a]pyridin-6-yl)-7-(4-hydroxypiperidin-4-yl)-4H-pyrido[1,2-a]pyrimidin-4-one, 2-(4-ethyl-6-methylpyrazolo[1,5-a]pyrazin-2-yl)-7-[(8aS)-hexahydropyrrolo[1,2-a]pyrazin-2 (1H)-yl]-4H-pyrido[1,2-a]pyrimidin-4-one, 2-(4-ethyl-6-methylpyrazolo[1,5-a]pyrazin-2-yl)-7-[(8 aR) hexahydropyrrolo[1,2-a]pyrazin-2 (1H)-yl]-4H-pyrido[1,2-a]pyrimidin-4-one, 7-[(3R)-3-(dimethylamino)pyrrolidin-1-yl]-2-(4-ethyl-6-methylpyrazolo[1,5-a]pyrazin-2-yl)-4H-pyrido[1,2-a]pyrimidin-4-one, 7-[(3S)-3-(dimethylamino)pyrrolidin-1-yl]-2-(4-ethyl-6-methylpyrazolo[1,5-a]pyrazin-2-yl)-4H-pyrido[1,2-a]pyrimidin-4-one, 2-(8-fluoro-2-methylimidazo[1,2-a]pyridin-6-yl)-7-[(8aS)-8a-methylhexahydropyrrolo[1,2-a]pyrazin-2 (1H)-yl]-4H-pyrido[1,2-a]pyrimidin-4-one, 2-(4-ethyl-6-methylpyrazolo[1,5-a]pyrazin-2-yl)-7-(4-ethylpiperazin-1-yl)-9-methyl-4H-pyrido[1,2-a]pyrimidin-4-one, 2-(4-ethyl-6-methylpyrazolo[1,5-a]pyrazin-2-yl)-7-[(8aS)-hexahydropyrrolo[1,2-a]pyrazin-2 (1H)-yl]-9-methyl-4H-pyrido[1,2-a]pyrimidin-4-one, 2-(2,8-dimethylimidazo[1,2-a]pyridin-6-yl)-7-(4-ethylpiperazin-1-yl)-4H-pyrido[1,2-a]pyrimidin-4-one, 2-(2,8-dimethylimidazo[1,2-a]pyridin-6-yl)-7-[(8aS)-hexahydropyrrolo[1,2-a]pyrazin-2 (1H)-yl]-4H-pyrido[1,2-a]pyrimidin-4-one, 2-(2,8-dimethylimidazo[1,2-a]pyridin-6-yl)-7-(8a-methylhexahydropyrrolo[1,2-a]pyrazin-2 (1H)-yl)-4H-pyrido[1,2-a]pyrimidin-4-one, 7-[(3R)-3-(dimethylamino)pyrrolidin-1-yl]-2-(4-ethyl-6-methylpyrazolo[1,5-a]pyrazin-2-yl)-9-methyl-4H-pyrido[1,2-a]pyrimidin-4-one, 2-(4-ethyl-6-methylpyrazolo[1,5-a]pyrazin-2-yl)-7-{[2-(morpholin-4-yl)ethyl]amino}-4H-pyrido[1,2-a]pyrimidin-4-one, 7-[(3R)-3-(dimethylamino)pyrrolidin-1-yl]-2-(2,8-dimethylimidazo[1,2-a]pyridin-6-yl)-4H-pyrido[1,2-a]pyrimidin-4-one, 7-[(3S)-3-(dimethylamino)pyrrolidin-1-yl]-2-(2, 8-dimethylimidazo[1,2-a]pyridin-6-yl)-4H-pyrido[1,2-a]pyrimidin-4-one, 2-(4,6-dimethylpyrazolo[1,5-a]pyrazin-2-yl)-7-[(3aR,6aS)-5-methylhexahydropyrrolo[3,4-c]pyrrol-2 (1H)-yl]-4H-pyrido[1,2-a]pyrimidin-4-one, salts, isotopologues, stereoisomers, racemates, enantiomers, diastereomers, or tautomers thereof.

The mRNA splicing modifier may comprise the following general structure of Formula II, as described in U.S. Pat. No. 9,969,754 B2, which is incorporated herein by reference in its entirety:

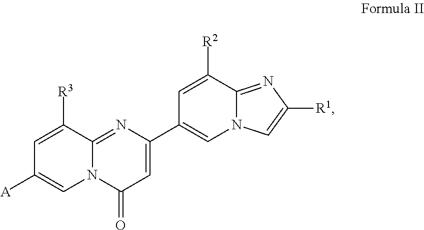

wherein R¹ is hydrogen or C₁₋₇alkyl, e.g. methyl; R² is hydrogen, cyano, C₁₋₇-alkyl, e.g. methyl, C₁₋₇-haloalkyl or C₃₋₈-cycloalkyl; R₃ is hydrogen, C₁₋₇alkyl, e.g. methyl, or C₃₋₈-cycloalkyl; A is N-heterocycloalkyl or NR¹²R¹³, wherein N-heterocycloalkyl comprises 1 or 2 nitrogen ring atoms and is optionally substituted with 1, 2, 3 or 4 substituents selected from R¹⁴; R¹² is heterocycloalkyl comprising 1 nitrogen ring atom, wherein heterocycloalkyl is optionally substituted with 1, 2, 3 or 4 substituents selected from R¹⁴, e.g. piperidinyl optionally substituted with 1, 2, 3 or 4 substituents selected from R¹⁴, R¹³ is hydrogen, C₁₋₇-alkyl or C₃₋₈-cycloalkyl; R¹⁴ is independently selected from hydrogen, C₁₋₇-alkyl, amino, amino-C₁₋₇-alkyl, C₃₋₈-cycloalkyl and heterocycloalkyl or two R¹⁴ together form C₁₋₇-alkylene; with the proviso that if A is N-heterocycloalkyl comprising only 1 nitrogen ring atom, then at least one R¹⁴ substituent is amino or amino-C₁₋₇-alkyl; or a pharmaceutically acceptable salt thereof. For example, R¹ may be hydrogen or C₁₋₇-alkyl; R² may be hydrogen, cyano, C₁₋₇-alkyl, C₁₋₇-haloalkyl or C₃₋₈-cycloalkyl; R³ may be hydrogen, C₁₋₇-alkyl, or C₃₋₈-cycloalkyl; A may be N-heterocycloalkyl comprising 1 or 2 nitrogen ring atoms, wherein N-heterocycloalkyl may optionally substituted with 1, 2, 3 or 4 substituents selected from R¹⁴; R¹⁴ may independently be selected from hydrogen, C₁₋₇-alkyl, amino, amino-C₁₋₇-alkyl, C₃₋₈-cycloalkyl and heterocycloalkyl or two R¹⁴ together form C₁₋₇-alkylene; with the proviso that if A is N-heterocycloalkyl comprising only 1 nitrogen ring atom, then at least one R¹⁴ substituent is amino or amino-C₁₋₇-alkyl; or a pharmaceutically acceptable salt thereof.

A may be

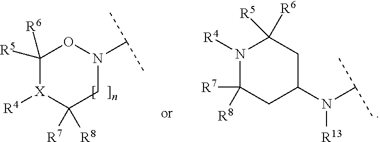

wherein X is N or CH; R⁴ is hydrogen, C₁₋₇-alkyl or —(CH₂)ₘ—NR⁹R¹⁰; R⁵ is hydrogen or C₁₋₇-alkyl; R⁶ is hydrogen or C₁₋₇-alkyl; R⁷ is hydrogen or C₁₋₇-alkyl; R⁸ is hydrogen or C₁₋₇-alkyl; R⁹ and R¹⁰ are independently selected from hydrogen, C₁₋₇-alkyl and C₃₋₈-cycloalkyl; R¹³ is hydrogen, C₁₋₇-alkyl or C₃₋₈-cycloalkyl; n is 0, 1 or 2; m is 0, 1, 2 or 3; or R⁴ and R⁵ together form C₁₋₇-alkylene; or R⁴ and R⁷ together form C₁₋₇-alkylene; or R⁵ and R⁶ together form C₂₋₇-alkylene; or R⁵ and R⁷ together form C₁₋₇-alkylene; or R⁵ and R⁹ together form C₁₋₇-alkylene; or R⁷ and R⁸ together form C₂₋₇-alkylene; or R⁷ and R⁹ together form C₁₋₇-alkylene; or R⁹ and R¹⁰ together form C₂₋₇-alkylene; with the proviso that if X is CH then R⁴ is —(CH₂)ₘ—NR⁹R¹⁰; and with the proviso that if X is N and R⁴ is —(CH₂)ₘ—NR⁹R¹⁰ then m is 2 or 3.

A may be

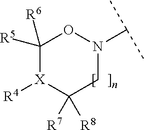

wherein X is N or CH; R⁴ is hydrogen, C₁₋₇-alkyl or —(CH₂)ₘ—NR⁹R¹⁰; R⁵ is hydrogen or C₁₋₇-alkyl; R⁶ is hydrogen or C₁₋₇-alkyl, e.g. methyl; R⁷ is hydrogen or C₁₋₇-alkyl, e.g. methyl; R⁸ is hydrogen or C₁₋₇-alkyl; R⁹ and R¹⁰ are independently selected from hydrogen, C₁₋₇-alkyl and C₃₋₈-cycloalkyl; n is 0, 1 or 2, e.g. 1; m is 0, 1, 2 or 3, e.g. 0; or R⁴ and R⁵ together form C₁₋₇-alkylene, i.e. propylene; or R⁴ and R⁷ together form C₁₋₇-alkylene; or R⁵ and R⁶ together form C₂₋₇-alkylene, e.g. ethylene; or R⁵ and R⁷ together form C₁₋₇-alkylene; or R⁵ and R⁹ together form C₁₋₇-alkylene; or R⁷ and R⁸ together form C₂₋₇-alkylene; or R⁷ and R⁹ together form C₁₋₇-alkylene; or R⁹ and R¹⁰ together form C₂₋₇-alkylene, e.g. butylene; with the proviso that if X is CH then R⁴ is —(CH₂)ₘ—NR⁹R¹⁰; and with the proviso that if X is N and R⁴ is —(CH₂)ₘ—NR⁹R¹⁰ then m is 2 or 3.

A may be selected from the group consisting of:

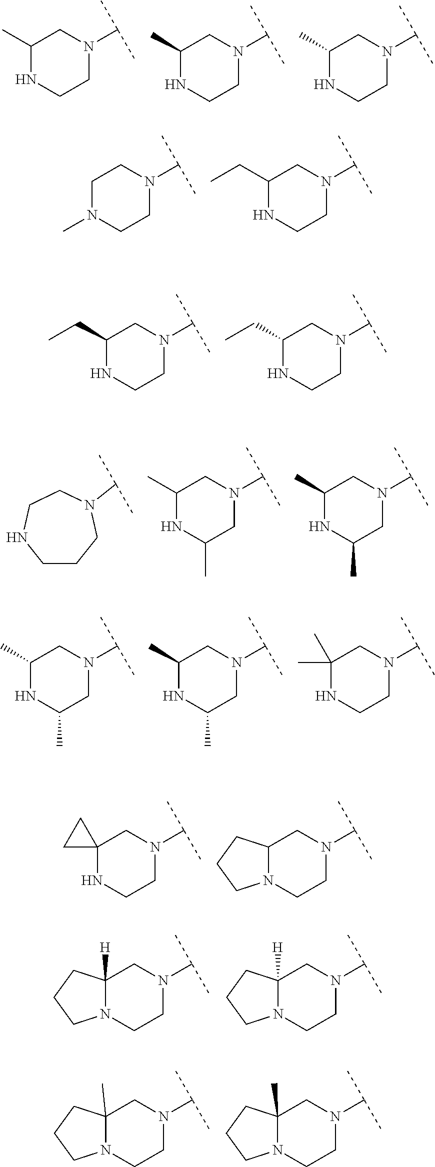

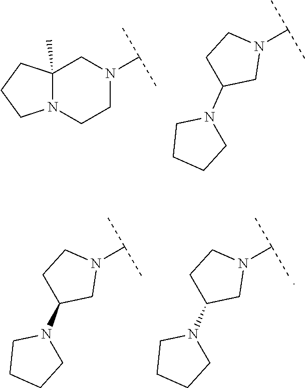

The mRNA splicing modifier comprising the structure of Formula II may be selected from the following: 2-(2-methylimidazo[1,2-b]pyridazin-6-yl)-7-(4-methylpiperazin-1-yl) pyrido[1,2-a]pyrimidin-4-one; 7-[(8aR)-3,4,6,7,8,8a-hexahydro-1H-pyrrolo[1,2-a]pyrazin-2-yl]-2-(2-methylimidazo[1,2-b]pyridazin-6-yl) pyrido[1,2-a]pyrimidin-4-one; 7-[(8aS)-3,4,6,7,8,8a-hexahydro-1H-pyrrolo[1,2-a]pyrazin-2-yl]-2-(2,8-dimethylimidazo[1,2-b]pyridazin-6-yl) pyrido[1,2-a]pyrimidin-4-one; 7-[(8aR)-3,4,6,7,8,8a-hexahydro-1H-pyrrolo[1,2-a]pyrazin-2-yl]-2-(2,8-dimethylimidazo[1,2-b]pyridazin-6-yl) pyrido[1,2-a]pyrimidin-4-one; 7-[(8aS)-8a-methyl-1,3,4,6,7,8-hexahydropyrrolo[1,2-a]pyrazin-2-yl]-2-(2,8-dimethylimidazo[1,2-b]pyridazin-6-yl) pyrido[1,2-a]pyrimidin-4-one; 7-[(8aR)-8a-methyl-1,3,4,6,7,8-hexahydropyrrolo[1,2-a]pyrazin-2-yl]-2-(2,8-dimethylimidazo[1,2-b]pyridazin-6-yl) pyrido[1,2-a]pyrimidin-4-one; 2-(2,8-dimethylimidazo[1,2-b]pyridazin-6-yl)-7-[(3S,5R)-3,5-dimethylpiperazin-1-yl]pyrido[1,2-a]pyrimidin-4-one; 2-(2,8-dimethylimidazo[1,2-b]pyridazin-6-yl)-7-[(3S)-3-methylpiperazin-1-yl]pyrido[1,2-a]pyrimidin-4-one; 2-(2,8-dimethylimidazo[1,2-b]pyridazin-6-yl)-7-[(3R)-3-methylpiperazin-1-yl]pyrido[1,2-a]pyrimidin-4-one; 7-(1,4-diazepan-1-yl)-2-(2,8-dimethylimidazo[1,2-b]pyridazin-6-yl) pyrido[1,2-a]pyrimidin-4-one; 2-(2-methylimidazo[1,2-b]pyridazin-6-yl)-7-[(3S)-3-methylpiperazin-1-yl]pyrido[1,2-a]pyrimidin-4-one; 2-(2-methylimidazo[1,2-b]pyridazin-6-yl)-7-[(3R)-3-methylpiperazin-1-yl]pyrido[1,2-a]pyrimidin-4-one; 7-(1,4-diazepan-1-yl)-2-(2-methylimidazo[1,2-b]pyridazin-6-yl) pyrido[1,2-a]pyrimidin-4-one; 7-[(3R,5S)-3,5-dimethylpiperazin-1-yl]-2-(2-methylimidazo[1,2-b]pyridazin-6-yl) pyrido[1,2-a]pyrimidin-4-one; 7-[(8aS)-3,4,6,7,8,8a-hexahydro-1H-pyrrolo[1,2-a]pyrazin-2-yl]-2-(2-methylimidazo[1,2-b]pyridazin-6-yl) pyrido[1,2-a]pyrimidin-4-one; 7-[(8aS)-8a-methyl-1,3,4,6,7,8-hexahydropyrrolo[1,2-a]pyrazin-2-yl]-2-(2-methylimidazo[1,2-b]pyridazin-6-yl) pyrido[1,2-a]pyrimidin-4-one; 7-[(8aR)-8a-methyl-1,3,4,6,7,8-hexahydropyrrolo[1,2-a]pyrazin-2-yl]-2-(2-methylimidazo[1,2-b]pyridazin-6-yl) pyrido[1,2-a]pyrimidin-4-one; 2-(2,8-dimethylimidazo[1,2-b]pyridazin-6-yl)-7-[(3R)-3-pyrrolidin-1-ylpyrrolidin-1-yl]pyrido[1,2-a]pyrimidin-4-one; 7-(4,7-diazaspiro[2.5]octan-7-yl)-2-(2-methylimidazo[1,2-b]pyridazin-6-yl) pyrido[1,2-a]pyrimidin-4-one; 7-(4,7-diazaspiro[2.5]octan-7-yl)-2-(2,8-dimethylimidazo[1,2-b]pyridazin-6-yl) pyrido[1,2-a]pyrimidin-4-one; 2-(2-methylimidazo[1,2-b]pyridazin-6-yl)-7-[(3R)-3-pyrrolidin-1-ylpyrrolidin-1-yl]pyrido[1,2-a]pyrimidin-4-one; 2-(2,8-dimethylimidazo[1,2-b]pyridazin-6-yl)-7-(3,3-dimethylpiperazin-1-yl) pyrido[1,2-a]pyrimidin-4-one; 7-(3,3-dimethylpiperazin-1-yl)-2-(2-methylimidazo[1,2-b]pyridazin-6-yl) pyrido[1,2-a]pyrimidin-4-one; 2-(2,8-dimethylimidazo[1,2-b]pyridazin-6-yl)-9-methyl-7-[(3S)-3-methylpiperazin-1-yl]pyrido[1,2-a]pyrimidin-4-one; 2-(2,8-dimethylimidazo[1,2-b]pyridazin-6-yl)-9-methyl-7-[(3R)-3-methylpiperazin-1-yl]pyrido[1,2-a]pyrimidin-4-one; 2-(2,8-dimethylimidazo[1,2-b]pyridazin-6-yl)-7-[(3R,5S)-3,5-dimethylpiperazin-1-yl]-9-methyl-pyrido[1,2-a]pyrimidin-4-one; 2-(2,8-dimethylimidazo[1,2-b]pyridazin-6-yl)-7-(3,3-dimethylpiperazin-1-yl)-9-methyl-pyrido[1,2-a]pyrimidin-4-one; 7-(4,7-diazaspiro[2.5]octan-7-yl)-2-(2,8-dimethylimidazo[1,2-b]pyridazin-6-yl)-9-methyl-pyrido[1,2-a]pyrimidin-4-one; 2-(2,8-dimethylimidazo[1,2-b]pyridazin-6-yl)-7-[(3S,5S)-3,5-dimethylpiperazin-1-yl]pyrido[1,2-a]pyrimidin-4-one; 2-(2,8-dimethylimidazo[1,2-b]pyridazin-6-yl)-7-[(3S)-3-pyrrolidin-1-ylpyrrolidin-1-yl]pyrido[1,2-a]pyrimidin-4-one; 2-(2-methylimidazo[1,2-b]pyridazin-6-yl)-7-[(3S)-3-pyrrolidin-1-ylpyrrolidin-1-yl]pyrido[1,2-a]pyrimidin-4-one; 7-[(3S,5S)-3,5-dimethylpiperazin-1-yl]-2-(2-methylimidazo[1,2-9-methyl-2-(2-methylimidazo[1,2-b]pyridazin-6-yl) pyrido[1,2-a]pyrimidin-4-one; b]pyridazin-6-yl)-7-[(3S)-3-methylpiperazin-1-yl]pyrido[1,2-a]pyrimidin-4-one; 9-methyl-2-(2-methylimidazo[1,2-b]pyridazin-6-yl)-7-[(3R)-3-methylpiperazin-1-yl]pyrido[1,2-a]pyrimidin-4-one; 7-[(3R,5S)-3,5-dimethylpiperazin-1-yl]-9-methyl-2-(2-methylimidazo[1,2-b]pyridazin-6-yl) pyrido[1,2-a]pyrimidin-4-one; 7-(3,3-dimethylpiperazin-1-yl)-9-methyl-2-(2-methylimidazo[1,2-b]pyridazin-6-yl) pyrido[1,2-a]pyrimidin-4-one; 7-(4,7-diazaspiro[2.5]octan-7-yl)-9-methyl-2-(2-methylimidazo[1,2-b]pyridazin-6-yl) pyrido[1,2-a]pyrimidin-4-one; 7-[(3S,5S)-3,5-dimethylpiperazin-1-yl]-9-methyl-2-(2-methylimidazo[1,2-b]pyridazin-6-yl) pyrido[1,2-a]pyrimidin-4-one; and 7-[(3R)-3-ethylpiperazin-1-yl]-2-(2-methylimidazo[1,2-b]pyridazin-6-yl) pyrido[1,2-a]pyrimidin-4-one; or a pharmaceutically acceptable salt thereof.

For example, the mRNA splicing modifier may be Risdiplam, or 7-(4,7-diazaspiro[2.5]octan-7-yl)-2-(2,8-dimethylimidazo[1,2-b]pyridazin-6-yl) pyrido[1,2-a]pyrimidin-4-one, comprising the following structure:

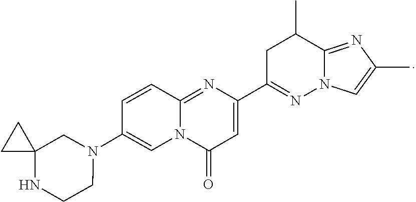 [SMILES: Cc1cn2c(n1)C(C)CC(c1cc(=O)n3cc(N4CCNC5(CC5)C4)ccc3n1)=N2]

Risdiplam may be commercially available as Evrysdi® (Genentech, Inc., San Francisco, CA).

The mRNA splicing modifier, the pharmaceutically-acceptable salt thereof, or the solvate thereof may be in the form of an oral solution. Oral solutions include an effective amount of the mRNA splicing modifier of at least about 0.25 mg/mL, at least 0.5 mg/mL, at least 0.75 mg/mL, at least 1 mg/mL, at least 2 mg/mL, from at least 0.25 mg/mL to at least 2 mg/mL, from at least 0.25 mg/mL to at least 1 mg/mL, from at least 0.5 mg/mL to at least 1 mg/mL, such as 0.75 mg/mL. In some cases, administration of the mRNA splicing modifier, the pharmaceutically-acceptable salt thereof, or the solvate thereof is at a dose from at least 0.15 milligrams per kilogram per day (mg/kg d) to at least 0.25 mg/kg d, or 5 milligrams per day (mg/d).

The therapeutic agent may be a gene therapy. For example, the gene therapy may be a gene replacement therapy therapeutic agent. The gene therapy may comprise a non-naturally occurring, non-replicating recombinant adeno-associated virus serotype 9 (AAV9) or lentivirus based vector comprising nucleic acid for expressing a human SMN1 transcript. Suitable AAV9 based vectors are described in U.S. Pat. No. 7,906,111 B2, which is incorporated by reference herein in its entirety.

The gene therapy may be Onasemnogene abeparvovec, commercially available as Zolgensma® (Novartis Gene Therapies, Inc., Basel, Switzerland). Onasemnogene abeparvovec is an AAV9-based drug comprising a gene for expressing an SMN1 transcript.

The gene therapy may be in the form of an intravenous infusion solution. The intravenous infusion solution of the gene therapy may include an effective amount of vector genomes (vg). For example, the intraveneous injectable solution of the gene therapy may include at least 0.5×10¹⁴ vg per kilograms of body weight (vg/kg), at least 1.0×10¹⁴ vg/kg, or at least 1.1×10¹⁴ vg/kg.

### EXAMPLES

For the sake of clarity, certain data provided in priority patent application, U.S. Provisional Patent Application No. 63/477,022 filed Dec. 23, 2023, which is hereby incorporated by reference in its entirety, is not shown herein.

### Example 1—Identification of Neurodevelopmental Disorder

The clinical and molecular spectrum of variants in GEMIN5 among 39 patients presenting with developmental delay, hypotonia, motor dysfunction, and cerebellar atrophy, suggested that GEMIN5 variants give rise to a distinct clinical phenotype. Biallelic variants in GEMIN5 were identified as a cause of a distinct neurological cerebellar ataxia syndrome, through altered snRNP complex assembly.

### Materials and Methods

Exome Sequencing of Families 1-22 (Cohort 1): Families 1, 11-13 and 15-18 were sequenced at GeneDx (Gaithersburg, MD). Using genomic DNA from the proband as well as parents and siblings, when available, the exonic regions and flanking splice junctions of the genome were captured using the SureSelect Human All Exon V4 (50 Mb), the Clinical Research Exome kit (Agilent Technologies, Santa Clara, CA) or the IDT x Gen Exome Research Panel v1.0. Massively parallel (NextGen) sequencing was done on an Illumina system with 100 bp or greater paired end reads. Reads were aligned to human genome build GRCh37/UCSC hg19 and analyzed for sequence variants using a custom-developed analysis tool. Exception is the patient from Family 13 whose sequencing was done using the Ataxia Xpanded panel and lacked full whole-exome sequencing (WES) analysis. Additional sequencing technology and variant interpretation protocol used were similar as Retter et al. (Genet Med., 2016, 18 (7): 696-704). For WES analysis of Family 3, in solution exome capture was performed using the SeqCap EZ Human Exome Kit v3.0 (Roche Nimblegen, USA) with 100-bp paired-end read sequences generated on a HiSeq2000 (Illumina, Inc. USA) in the Centro Nacional de Análisis Genómico in Barcelona (CNAG). Single variants and insertions/deletions (indels) were identified using the GATK's best practices for germline SNP & Indel discovery in WES and annotated by the Annovar software. Copy number variation (CNV) was analysed by R package Exome Depth.

The general assertion criteria for variant classification are publicly available on the GeneDx ClinVar submission page. A subset of the GEMIN5 patients were found through GeneMatcher Sobreira et al., Hum Mutat, 2015, 36 (4): 425-431 and Sobreira et al., Hum Mutat, 2015, 36 (10): 928-930). All the variants were annotated by using the GEMIN5 NP_056280.2 reference transcript in GnomAD and the other databases to estimate the allelic frequency. The damaging index of GEMIN5 variants was determined by using various in-silico prediction tools such as Polyphen2 (Adzuebei et al., Nat Methods, 2010, 7 (4): 248-249), Provean (Choi et al., PLoS One, 2012, 7 (10): e46688), SNAP2 (Bromberg et al., Nucleic Acids Res, 2007, 35 (11): 3823-3835), MUpro (Cheng et al., Proteins, 2006, 62 (4): 1125-1132), PhD SNP (Capriotti et al., Nucleic Acids Res, 2017, 45 (W1): W247-W252), and SIFT (Ng et al., Nucleic Acids Res, 2003, 31 (13): 3812-3814).

Exome Sequencing of Families 23-29 (cohort 2): WES was performed at different genetic centers using next-generation sequencing techniques and all variants were confirmed via Sanger sequencing with standard methods.

For Family 23, the trio was sequenced at the Yale Center for Genome Analysis (YCGA). Genomic DNA was captured using an IDT xGen exome kit followed by Illumina DNA sequencing. WES data was processed using two independent pipelines at the Yale School of Medicine and Phoenix Children's Hospital. At each site, sequence reads were mapped to the reference genome (GRCh37) with BWA-MEM and further processed using GATK Best Practice workflows, which include duplication marking, indel realignment, and base quality recalibration. Single nucleotide variants and small indels were called with GATK HaplotypeCaller and annotated using ANNOVAR, dbSNP (v138), 1000 Genomes (August 2015), NHLBI Exome Variant Server (EVS), and the Exome Aggregation Consortium v3 (ExAC). Rare deleterious missense variants and LOF variants (stop-gain, stop-loss, frameshift insertions/deletions, canonical splice site, and start-loss) were selected. MetaSVM and Combined Annotation Dependent Deletion (CADD v1.3) algorithms were used to predict deleteriousness of missense variants (MetaSVM-deleterious or CADD≥20).

For Family 24, the trio was analyzed at the Genomic Sequencing Platform Seqoia (Paris) as follows: preparation of the libraries using NEBNext® Ultra II End repair/A-tailing module & Ligation module (New England Biolabs®), whole genome sequencing on a NovaSeq 6000® (Illumina®) using 2×150 paired-end sequencing, high quality reads mapping against the human reference genome (hg38), variant calling with GATK4 v4.1.7.0 (Broad Institute), annotation with SNPeff (4.3t) et SnpSift (4.3t).

For Family 26, the patients were recruited and consent for participation in the study was obtained according to the Declaration of Helsinki and approved by the Central Oxford Research Ethics Committee and the Research and Development Department of the Oxford Radcliffe Hospitals NHS Trust, Oxford. All patients, or their parents, provided written consent for the study.

For Family 27, the proband and his parents provided written informed consent to a study approved by the Mayo Clinic Institutional Review Board.

For Family 29, WES was performed in the two affected siblings, using DNA from peripheral blood leukocytes. The exome was enriched using the SureSelectXT V5 exome kit (Agilent, Böblingen, Germany), and sequenced on a HiSeq2500 sequencer (Illumina, San Diego, CA, USA). High quality reads were mapped against the human reference genome (hg19) and variants were called following Genome Analysis Tool Kit version 3.3.0 best practices recommendations (McKenna et al., Genome Res, 2010, 20 (9): 1297-1303; Ma et al., Bioinformatics, 2013, 29 (18): 2261-2268). After annotation with Annovar, filtration and downstream analysis was done with FILTUS (Vigeland et al., Bioinformatics, 2016, 32 (10): 1592-1594). The identified GEMIN5 variants were validated by Sanger sequencing.

The damaging index of GEMIN5 variants was determined by using various in silico prediction tools such as Polyphen2, Provean, SNAP2, MUpro, PhD SNP, and SIFT (Cheng et al., 2006; Ng et al.; Bromberg et al., Nucleic Acids Res, 2007, 35 (11): 3823-3835; Mi et al., Nucleic Acids Res, 2010, 38 (Database issue): D204-D210; Sim et al., Nucleic Acids Res, 2012, 40 (Web Server issue): W452-457; Choi et al.). Candidate variants were validated by sanger sequencing and tested for cosegregation in all family members whenever samples were available.

All the variants were annotated by using the GEMIN5 NP_056280.2 reference transcript to estimate the allelic frequency in GnomAD and the other databases. The effect of GEMIN5 variants on its structure and folding was predicted by using molecular graphics tool: PyMOL.

CRISPR/Cas9-mediated generation of iPSCs

Plasmid generation: The iPSC lines were generated by a previously published CRISPR/Cas9 technique (Wang et al., Stem Cell Res, 2019, 36:101391). Following sgRNA identification for the site of interest using the CRISPOR design tool (Haeussler et al., Genome Biol, 2016, 17 (1): 148), the sgRNA sequences were cloned into the pLentiCRISPR-V2 plasmid from the laboratory of Feng Zhang (AddGene #52961) following the protocol provided with the plasmid (Shalem et al., Science, 2014, 343 (6166): 84-87; Sanjana et al., Nat Methods, 2014, 11 (8): 783-784).

Electroporation, selection, and growth of edited iPSCs: Human ESCs or iPSCs were cultured in hPSC medium on mouse embryonic fibroblast (MEF) feeder cells with Rho Kinase (ROCK)-inhibitor (1.0 μM, Calbiochem, H-1152P) for 24 hours prior to electroporation (Sanjana et al., 2014). Cells were digested by TrypLE express Enzyme (Life Technologies) for 3-4 minutes, washed two times with DMEM/F12, and harvested in hPSC medium with 1.0 μM ROCK-inhibitor. Cells were dispersed into single cells, and 1×10⁷ cells were electroporated with appropriate combination of plasmids in 500 microliters (μl) of Electroporation Buffer (KCl 5 millimolar (mM), MgCl₂ 5 mM, HEPES 15 mM, Na₂HPO₄ 102.94 mM, NaH₂PO₄ 47.06 mM, pH=7.2) using the Gene Pulser Xcell System (Bio-Rad) at 250 Volts (V), 500 microfarad (μF) in 0.4 centimeter (cm) cuvettes (Phenix Research Products). Cells were electroporated in a cocktail of 15 micrograms (μg) of the pLentiCRISPRV2-Gemin5 sg1fwd plasmid and 100 μL of a 10 micromolar (μM) ssODN targeting the Gemin5 locus. This ssODN was non-complementary to the sgRNA sequence and consisted of 141 nucleotides—70 nucleotides upstream and 70 nucleotides downstream of the targeted base pair. Following electroporation, cells were plated on MEF feeders in 1.0 μM ROCK inhibitor. At 24-and 72-hours post-electroporation, cells were treated with puromycin (0.33 micrograms per milliliter (μg/ml), Invivogen, ant-pr-1) to select for cells containing the pLentiCRISPRV1-Gemin5 sg1fwd plasmid. Concurrent with puromycin treatment, the cells were fed with MEF-conditioned hPSC media containing 1.0 μM ROCK inhibitor. After removal of the puromycin at 96 hours, cells were cultured in MEF-conditioned hPSC media until colonies were visible.

Genotyping: Single-cell colonies were manually selected and mechanically disaggregated. Genomic DNA was isolated from a portion of these colonies using QuickExtract DNA Extraction Solution 1.0 (Epicentre). Genotyping primers were designed flanking the mutation site, allowing amplification of this region using Q5 polymerase-based PCR (NEB). PCR products were identified via agarose gel and purified using a Zymoclean Gel DNA Recovery Kit (Zymo Research). Clones were submitted to Quintara Biosciences for Sanger sequencing to identify clones with the proper genetic modification.

Off-target analysis: To identify whether the CRISPR-Cas9 system produced any non-specific genome editing, suspected off-target sites were analyzed for genome modification. Using the five highest-likelihood off-target sites predicted by the CRISPOR algorithms (Doench et al., Nat Biotechnol, 2016, 34 (2): 184-191), genotyping primers were designed to amplify these regions via Q5-polymerase PCR. PCR products were identified via agarose gel, purified using a Zymoclean Gel DNA Recovery Kit, and submitted to Quintara Biosciences for Sanger sequencing.

Generation of induced pluripotent stem cells (iPSCs) from peripheral blood: Peripheral blood mononuclear cells (PBMCs) were isolated from whole blood processed and reprogrammed into iPSCs by the Stem Cell Core Facility at Northwestern to generate clonal iPSC lines from patient blood. All samples were banked and then processed together to minimize variability due to batch effects. When a low number of PBMCs were isolated from limited patient samples, erythroid cells were expanded using SFEM II media supplemented with cytokines SCF, IL-3, and EPO for subsequent iPSC reprogramming. When expanded to a sufficient number, a non-integrating Sendai viral-based reprogramming kit (CytoTune 2.0 from ThermoFisher) was used to introduce the four “Yamanaka reprogramming factors”, OCT4, SOX2, KLF4, and MYC. Reprogrammed iPSCs were expanded on plates coated with hESC-qualified matrigel (Corning) and grown in mTeSR plus (Stem Cell Technologies). Clonal iPSC-like colonies were selected, expanded, and characterized to pass several quality control standards. At least three colonies for each line were selected after meeting our criteria for morphology, growth, sterility, and iPSC marker expression. Cells were expanded and analyzed to ensure>80% of colonies are free of differentiated cells and readily expand following passaging. Routine testing was performed on each clonal line to ensure they were free of mycoplasma contamination; karyotype analysis was performed to ensure cells were free of abnormalities, and STR analysis was performed to validate the identity of the cells.

Cell culture and differentiation of iPSCs into neuronal cells: The iPSCs were differentiated into neuronal cells using a similar protocol as (Ortega et al., Neuron, 2020, 106(1): 90-107 e13). The iPSCs were cultured and maintained in mTeSR™ 1 media (STEMCELL technologies) on Matrigel coated plates. For differentiation, approximately 0.6 million cells were plated and let to grow for up to 80-90% confluency in mTeSR™ 1 for two days. For the first phase of differentiation, the confluent iPSC cells were grown for 6 days in N2B27 Neurobasal/DMEM-F12 medium (1:1 v/v) containing 1% N₂ (Gibco, 17502-048), 2% B27 (Gibco, 17054-044), 1% Glutamax (Gibco), and non-essential amino acids (NEAA) (Gibco, 11140050) along with 10 μM SB431542 (STEMCELL technologies), 0.1 μM LDN (Sigma SML0559), 1 μM retinoic acid (RA) (Sigma R2625), 1 μM smoothened agonist (SAG, Cayman chemicals 11914). For day 7 to 14, cells were grown in N2B27 media supplemented with 1 μM RA, 1 μM SAG, 10 μM DAPT (Cayman, 13197),16 μM SU5406 (Cayman, 131825). On day 14, cells were dissociated using TrypLE/DNase I (Invitrogen) and cultured on poly-ornithine and laminin coated coverslips or plates in neuronal media containing neurobasal medium, N2, B27, 0.4 milligrams per milliliter (mg/ml) ascorbic acid (Sigma, A4403), 10 μg/ml human brain-derived neurotrophic factor (BDNF) (Peprotech, 45002), 10 μg/ml glial cell derived neurotrophic factor (GDNF) (Peprotech, 45010), 10 μg/ml ciliary neurotrophic factor (CNTF) (Peprotech, 45013), 1% Glutamax, and NEAA. The cells were differentiated into neurons for 28 days and processed for subsequent Immunofluorescence and WB analysis.

Immunofluorescence (IF): For IF, the neurons were fixed in 4% paraformaldehyde (PFA) for 10 min and blocked in 0.1% Triton-X in PBS and 5% normal goat serum for 10 minutes. The cells were treated overnight with the following antibodies: rabbit anti-GEMIN5 (Millipore Sigma HPA037393, 1:1,000), mouse anti-GEMIN2 [2E17](abcam ab6084, 1:500), mouse anti-GEMIN6/SIP2, (abcam ab88290, 1:500) rabbit anti-GEMIN4 (NOVUS Biologicals NB110-40591, 1:500), mouse anti-GEMIN3, clone 12H12 (Millipore Sigma 05-1533, 1:500), mouse anti-SMN (BD transduction 610646, 1:1,000), rabbit anti-U1A (NOVUS Biologicals NBP2-53095, 1:2,000), chicken anti-beta-III Tubulin (NOVUS Biologicals NB100-1612-1:1,000), goat anti-MAP2 (Synaptic System-188 004, 1:1,000), and mouse anti-Ubiquitin. Alexa fluor-488,-568 and 647 secondary antibodies were used from Invitrogen. The cells were mounted using fluoroshield™ with DAPI (Sigma) and images were taken at 60×using Nikon A1-T216.3 confocal microscope.

Western Blot (WB) analysis: Differentiated neurons and HEK293T cells were dissociated in TrypLE/DNase and cells were pelleted down at 250 times gravity (x g) at room temperature. The cells were washed with phosphate buffered saline (PBS) and lysed in RIPA buffer containing 150 mM NaCl, 50 mM NaF, 2 mM EDTA, 0.2 mM Na orthovanadate, 1% sodium deoxycholate, 2 mM DTT, 1% NP40, 0.1% SDS, and protease inhibitor (Roche 11836170001). The lysates were sonicated and centrifuged at 10,000×g for 15 min at 4° C. The concentration of proteins in the supernatant were measured by Pierce™ BCA protein assay kit (Thermo Scientific 23227). Equal concentration of supernatant was boiled with 1×Laemmli buffer and the proteins were separated using 4-12% NuPage bis-Tris gel (Novex/Life Technologies). Proteins were transferred onto nitrocellulose (Invitrogen IB23001) using the iBlot2 (Life Technologies 13120134). The blots were blocked in 2.5% QuickBlocker reagent (EMB Millipore WB57-175GM) and probed overnight with the following antibodies: mouse anti-tubulin (SIGMA, 1:10,000) anti-GEMIN5 (GenTex GTX130498, 1:1,000), mouse anti-GEMIN2 [2E17](1:2,000), mouse anti-GEMIN6/SIP2 (1:5,000) rabbit anti-GEMIN4 (1:2,000), mouse anti-GEMIN3, clone 12H12 (1:1,000), mouse anti-SMN (1:5,000), and rabbit anti-U1A (NOVUS Biologicals NBP2-53095, 1:2,000).

For immunoprecipitation, lysates were prepared from HEK cells expressing HA tagged GEMIN5, L1068P and H913R in 10 mM Tris-Hcl (pH 7.5), 100 mM NaCl, 2.5 mM MgCl₂, 0.1% NP40, 2 mM DTT, 2.5 mM sodium orthovanadate and 1×protease inhibitor cocktail (Invitrogen). The lysates were incubated with anti-HA antibody overnight at 4° C. and the HA-protein complex was pulled down by incubating with Protein A Dynabeads (Invitrogen) for 3 hrs at 4° C. The proteins were denatured and probed for anti-HA, anti-GEMIN4, anti-GEMIN3, and SMN. Secondary antibodies used were anti-mouse DYLight 800 and anti-rabbit 680 (Invitrogen, 1:10,000). The blots were imaged using Licor imager (Odyssey CLx). All the blots were run in triplicates and the integrated band densities were calculated using image studio software (Licor).

mRNA stability and Gene expression analysis: RNA was isolated from iPSC-derived differentiated neurons by using the PureLink™ RNA mini kit (Invitrogen), following the manufacturer's instructions. Around 500 nanograms (ng) of RNA was used to synthesis cDNA with oligodT by using iScript™ Reverse Transcription kit (BioRad). Quantitative PCR was performed in a 20 μl reaction in 7300 Real Time PCR machine (Applied Biosystems) using custom design 5′ 6-FAM/ZEN/3′ IBFQ IDT PrimeTime Assay set (Appendix table 1). Gene expression levels (Ct values) were normalized with GAPDH as an internal control. For qPCR validation in flies, RNA was isolated from three whole flies expressing the RNAi by using TRizol and gene expression was normalized with Tubulin. mRNA decay was designed as mentioned above by using relative transcript abundance after 0, 1, 2, 4, 6, and 8 hours of actinomycin D (Sigma A1410) treatment.

In vitro snRNP assembly assay: Cytoplasmic extracts from the differentiated neurons were prepared using NE-PER nuclear and cytoplasmic extraction kit (Thermo Scientific 78835) and the protein concentrations were measured by Pierce™ BCA protein assay kit. UlsnRNAs were transcribed from gel-eluted and linearized DNA template by in vitro transcription using T7 RNA polymerase and m7G cap analogue. pCp-Cy3 (Cytidine-5′-phosphate-3′-(6-aminohexyl) phosphate) (Jena Bioscience) was transferred to the 3′-hydroxyl group on UlsnRNA by T4 RNA ligase (ThermoFisher). The snRNP assembly reaction was carried out by incubating 5 μg of pCp-Cy3 labelled UlsnRNAs with 50 μg of cytoplasmic extract, 10 μM tRNA and 2.5 mM ATP at 30° C. for one and half hours. The reaction mix were loaded onto native 6% TBE polyacrylamide gel (Novex/Life Technologies). The gel was run at 150 V at 4° C. and was imaged using Licor imager.

RNA sequencing (RNAseq): RNA was isolated from iPSC-derived differentiated neurons with homozygous and heterozygous GEMIN5 His913Arg variants by using the PureLink™ RNA mini kit (Invitrogen). RNAseq was performed using the BGISEQ-500 platform combining the DNA nanoball-based nanoarrays and stepwise sequencing using Combinational Probe Anchor Synthesis Sequencing Method. Reads were mapped to human reference genome (hg19) using Bowtie2, and gene expression level were calculated with RSEM. Between the samples Pearson correlation was calculated using cor and the differentially expressed genes with the fold Change≥1.5 adjusted P value≤0.05 were selected. The DEGs with a false discovery rate (FDR) of not larger than 0.01 were used for Gene Ontology (GO) functional enrichment using phyper. Statistical analysis was performed by using R. Differential splicing detection was done using the NBSplice package in Bioconductor/R. The expression matrix at the transcript/isoform level generated using RSEM was used as the input (Li et al., BMC Bioinformatics, 12:323; Merino et al., J Biomed Inform, 2020, 103:103378). Negative binomial generalized linear models were fitted at the gene level and allow the estimation of significant differences in isoforms relative expression values between the biological conditions. The significance threshold is set at 5%. The Database for Annotation, Visualization and Integrated Discovery (DAVID) v6.8 was used to functionally annotate the differentially spliced genes into different pathways (Huang et al., Nucleic Acids Res, 2009 (a), 37 (1): 1-13; Huang et al. Nat Protoc, 2009b, 4 (1): 44-57). The Upkeyword pathways were plotted for genes with FDR<0.05.

For comparing the SMA sequencing data with GEMIN5 (His913Arg), both datasets were processed in a similar way. The SMA (SMN1Exon7del) RNAseq data was obtained from Answer ALS, a large-scale resource for sporadic and familial ALS combining clinical data with multi-omics data from induced pluripotent cell lines. Quality controlled FASTQ files were aligned to the Ensemble Human reference genome (hg38) using STAR aligner (version 2.5.1). HTSeq-count were used to generate counts of reads uniquely mapped to annotated genes using the GRCh38 annotation gtf file (Anders et al., Bioinformatics, 2015, 31 (2): 166-169). Differential gene expression analysis between the different conditions was done using DESeq251 using a model based on the negative binomial distribution. The resulting P-values were adjusted using the Benjamini and Hochberg's approach for controlling the false discovery rate, and differentially expressed genes were determined at the 5% threshold. Gene set enrichment analysis was used to assess the statistical enrichment of gene ontologies, and pathways (Subramanian et al., Proc Natl Acad Sci USA, 2005, 102 (43): 15545-15550).

Larval eclosion assay: UAS-rigor mortis KK RNAi lines (VDRC 105403) were crossed with inducible driver Tubulin-GS-Gal4, at 28° C. on food mixed with 1 mM RU486 (Cayman Chemicals) for inducing transgene expression. The larvae were monitored from the 1^{st} instar stage until they eclosed and become adults. The images of each developmental stage were taken using a Leica M205C dissection microscope equipped with a Leica DFC450 camera.

Motor dysfunction assays: UAS-rigor mortis KK RNAi lines (VDRC 105403) were crossed with ubiquitous inducible driver, Tubulin Gene switch (TubGS)-Gal4. Day 1 adults from the F1 progeny were collected every 24 hours and moved to standard media mixed with 20 mM RU486 at 28° C. Locomotion was assessed using the RING assay (Gargano et al., Exp Gerontol, 2005, 40 (5): 386-395; Nichols et al., J Vis Exp, 2012, 61). Briefly, flies were transferred, without anesthetization, into plastic vials and placed in the RING apparatus. The vials were tapped down against the bench and the climbing was recorded on video at day 20. Quantifications were performed manually by a third party in a blinded manner.

For studying neuromuscular junctions (NMJs) defects, 3^{rd} instar larvae expressing Rig mortis RNAi were dissected, and fixed by using 4% formaldehyde. The RNAi was expressed using TubGS-Gal4 by growing the 1^{st} instar larvae on 1 mM RU486 at 28° C. until they reach the 3^{rd} instar stage. The larvae (n=4) were probed with mouse anti-Horseradish Peroxidase (HRP), a presynaptic neuronal marker to identify the neuromuscular junctions, for overnight at 4° C. On the next day, the larvae were washed with 0.1% TBST and stained with goat anti-mouse Alexa fluor-568 secondary antibody. The larvae were mounted with fluoroshield™ (Sigma) and images were taken at 60×using Nikon A1-T216.3 confocal microscope.

Life span assay: Lifespan assay was performed on day 1 adult females. Female flies expressing the transgene for Rig mortis RNAi by using TubGS-Gal4 were separated and transferred to experimental vials containing fly food mixed with RU486 (20 mM) at a density of 25 flies per vial (n>100). Deaths were scored every other day and flies were transferred to fresh food three times a week.

Gemin5 Knock Out Mice: Creation of the Gemin5 knockout (KO) mice in the C₅₇BL/6N background was carried out at the MRC Harwell Institute through the International Mouse Phenotyping Consortium (IMPC). Using CRISPR/Cas9 and the EUCOMM/KOMP-CSD allele structure, a premature stop codon was introduced into exon 7 of the Mus muculus Gemin5 gene, resulting in a null Gemin5 allele. Heterozygous Gemin5 knockout mice (C₅₇BL/6NTac-Gemin5em1 (IMPC) H/H) were confirmed via short range PCR at the MRC Harwell Institute.

Statistical analysis: Statistical analysis was done on GraphPad Prism using one-way ANOVA (analysis of variance) followed by a Bonferroni or Tukey post hoc test for comparison between two or more groups. To compare two experimental conditions, two-tailed non-parametric Mann-Whitney U test was performed. For analysis of mRNA stability, normalized values for 0, 1, 2, 4, 6, and 8 hours were fitted to the non-linear regression of one phase-exponential decay model and half-lives were calculated using the equation, t1/2=ln (2)/k.

### Results and Discussion

Thirty-eight additional patients were identified after the index p. (Leu1068Pro) patient in 29 unrelated families with biallelic GEMIN5 variants (Cohort 1-Families 1-22 and Cohort 2-families 23-29) (Tables 1-4). All patients showed motor predominant developmental delays and were diagnosed within the first 2 years of life. Patients 4, 5, and 6 (Family 3 and 4) presented with severe hypotonia at birth and were evaluated for SMA. These three patients passed away before 3 years of age. Most other patients had easily elicitable reflexes and did not fit the classical phenotype of SMA. While cognitive and speech delays were seen in most patients, the development delay was predominantly motor (Table 2). No motor or cognitive regression was found in any of the patients. 23 of the 30 patients in families 1-22 (cohort 1) had central hypotonia, however, the appendicular tone was variable and included concomitant spasticity with brisk reflexes in 13 of the 30 patients. All ambulatory patients had a gait ataxia. 16 of the 30 patients had an electromyography (EMG) and Nerve Conduction Velocity (NCV) where 10 of these suggestive off neuropathic as opposed to motor neuron disease. 15 of the patients had a static phenotype, with 6 patients experiencing a progressive phenotype. Data on the clinical progression of the remaining 9 patients from cohort 1 was unavailable.

TABLE 1
Clinical summary of GEMIN5 families 1-22

| | Family | | | | | | | | | | | | | | |
|---|---|---|---|---|---|---|---|---|---|---|---|---|---|---|---|
| | 1 | 2 | | 3 | 4 | | 5 | 6 | | 7 | | 8 | | 9 | |
| | Patient number | | | | | | | | | | | | | | |
| | 1 | 2 | 3 | 4 | 5 | 6 | 7 | 8 | 9 | 10 | 11 | 12 | 13 | 14 | 15 |
| | Gender | | | | | | | | | | | | | | |
| | F | F | F | M | M | M | M | M | M | F | F | M | M | M | M |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | | | | | | | | | | | | | | | |
| [symbol] | Y | Y | Y | Y | Y | Y | Y | Y | Y | Y | Y | Y | Y | Y | Y |
| [symbol] | N | N | N | N | N | N | N | N | N | N | N | N | N | N | N |
| [symbol] | Y | Y | Y | Y | Y | Y | Y | Y | Y | Y | Y | Y | Y | Y | Y |
| [symbol] | Y | Y | Y | Y | [symbol] | [symbol] | Y | Y | Y | Y | Y | Y | Y | Y | Y |
| [symbol] | Y | Y | Y | Y | [symbol] | [symbol] | Y | Y | Y | Y | Y | Y | Y | Y | Y |
| [symbol] | | | | | | | | | | | | | | | |
| [symbol] | Y | Y | [symbol] | [symbol] | [symbol] | [symbol] | Y | Y | Y | Y | Y | [symbol] | [symbol] | Y | [symbol] |
| [symbol] | N | Y | Y | N | N | N | N | Y | Y | Y | Y | N | N | N | N |
| [symbol] | Y | Y | Y | Y | Y | Y | Y | Y | Y | | | Y | Y | Y | Y |
| [symbol] | N | N | B | A | A | A | [symbol] | B | B | [symbol] | [symbol] | N | N | [symbol] | [symbol] |
| [symbol] | | | | | | | | | | | | | | | |
| [symbol] | Y | Y | Y | Y | Y | Y | Y | Y | Y | Y | Y | Y | Y | Y | Y |
| [symbol] | | | | | | | | | | | | | | | |
| [symbol] | [symbol] | [symbol] | [symbol] | [symbol] | [symbol] | [symbol] | [symbol] | [symbol] | [symbol] | [symbol] | [symbol] | [symbol] | [symbol] | | |

| | | | | | | | | | |
|---|---|---|---|---|---|---|---|---|---|
| [symbol] | [symbol] | [symbol] | [symbol] | [symbol] | [symbol] | [symbol] | [symbol] | [symbol] | [symbol] |

Clinical summary of GEMIN5 families 1-22

| | Family | | | | | | | | | | | | | | |
|---|---|---|---|---|---|---|---|---|---|---|---|---|---|---|---|
| | 10 | 11 | 12 | | 13 | 14 | 15 | 16 | 17 | | 18 | 19 | 20 | 21 | 22 |
| | Patient number | | | | | | | | | | | | | | |
| | 16 | 17 | 18 | 19 | 20 | 21 | 22 | 23 | 24 | 25 | 26 | 27 | 28 | 29 | 30 |
| | Gender | | | | | | | | | | | | | | |
| | F | M | F | M | M | M | M | F | M | M | F | F | M | M | F |
| [symbol] | [symbol] | | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | | [symbol] | [symbol] | [symbol] | [symbol] | [symbol] | [symbol] | [symbol] | [symbol] |
| [symbol] | | | | | | | | | | | | | | | |
| [symbol] | Y | Y | Y | Y | Y | Y | Y | Y | Y | Y | Y | Y | Y | Y | Y |
| [symbol] | N | N | N | N | N | N | N | N | N | N | N | N | N | N | N |
| [symbol] | Y | Y | Y | Y | Y | Y | Y | Y | Y | Y | Y | Y | Y | Y | Y |
| [symbol] | Y | Y | Y | Y | Y | Y | | Y | Y | Y | N | Y | Y | Y | Y |
| [symbol] | Y | Y | Y | Y | Y | Y | | Y | Y | Y | N | Y | Y | Y | Y |
| [symbol] | | | | | | | | | | | | | | | |
| [symbol] | Y | Y | Y | Y | Y | Y | [symbol] | Y | Y | Y | [symbol] | [symbol] | Y | [symbol] | Y |
| [symbol] | N | | Y | Y | Y | Y | N | N | Y | Y | N | Y | N | N | N |
| [symbol] | Y | Y | Y | Y | Y | Y | Y | Y | N | N | Y | Y | Y | Y | N |
| [symbol] | B | | B | B | B | B | B | [symbol] | B | B | A | B | B | N | N |
| [symbol] | | | | | | | | | | | | | | | |
| [symbol] | Y | Y | Y | Y | Y | Y | Y | Y | Y | Y | Y | Y | Y | Y | Y |
| [symbol] | | | | | | | | | | | | | | | |
| [symbol] | | [symbol] | [symbol] | [symbol] | [symbol] | [symbol] | | | [symbol] | [symbol] | [symbol] | [symbol] | [symbol] | [symbol] | [symbol] |

| | | | | | | | | | | | | | |
|---|---|---|---|---|---|---|---|---|---|---|---|---|---|
| [symbol] | [symbol] | [symbol] | [symbol] | [symbol] | [symbol] | [symbol] | [symbol] | [symbol] | [symbol] | [symbol] | [symbol] | [symbol] | [symbol] |

Description of GEMIN5 variants in families 1-22.

| | GEMIN5 Variant | GEMIN5 Variant | | |
|---|---|---|---|---|
| | (cDNA) | (protein) | Allele frequency | Number of |
| Family | (NM_015465.5) | (NP_056280.2) | (heterozygous) | homozygous |
| 1 | c.3203T > C | Leu1068Pro | 3.98e−5 | 0 |
| 2 | c.3019G > A | Ala1007Thr | 0 | 0 |
| 3 | c.2768A > C | His923Pro | 0 | 0 |
| 4 | c.2738A > G | His913Arg | 0 | 0 |
| 5 | c.2112C > G | Asp704Glu | 0 | 0 |
| 6 | c.2049C > T | Gly683Asp | 0 | 0 |
| 7 | c.2995T > C | Ser1000Pro | 0 | 0 |
| 8 | c.2995T > C | Ser1000Pro | 0 | 0 |
| 9 | c.1831G > A | Val611Met, | 8.07e−6 | 0 |
| | c.4091A > C | His1364Pro | 0 | 0 |
| 10 | c.3857A > G | Tyr1286Cys, | 3.99e−6 | 0 |
| | 2510-2A > T | 2510-2A > T | 0 | 0 |
| | (inversion) | (inversion) | | |
| 11 | c.2962A > T | Ile988Phe | 9.15e−5 | 0 |
| | c.4100T > C | Leu1367Pro | 3.89e−5 | 0 |
| 12 | c.4100T > C | Leu1367Pro | 3.89e−5 | 0 |
| | c.3057C > A | Asp1019Glu | 0 | 0 |
| 13 | c.3844T > C | Tyr1282His | 4.01e−6 | 0 |
| | c.217T > C | Ser73Pro | 0 | 0 |
| 14 | c.3356T > C | Leu1119Ser | 0 | 0 |
| | c.1602C > A | Tyr534Ter | 0 | 0 |
| 15 | c.2962A > T | Ile988Phe | 9.15e−5 | 0 |
| | c.1856delA | Glu619GlyfsTer8 | 1.23e−5 | 0 |
| 16 | c.314A > G | His105Arg | 0 | 0 |
| | c.2768A > C | His923Pro | 0 | 0 |
| 17 | c.2962A > T | Ile988Phe | 9.15e−5 | 0 |
| | c.3930_3933delCTCT | Ser1311LeufsTer7 | 3.98e−6 | 0 |
| 18 | c.485A > G | His162Arg | 0 | 0 |
| | c.4100T > C | Leu1367Pro | 3.89e−5 | 0 |
| 19 | c.282G > A | Trp94ter | 0 | 0 |
| | c.3856T > A | Tyr1286Asn | 0 | 0 |
| 20 | c.1081-2A > G (Splice | 1081-2A > G (splice | 0 | 0 |
| | acceptor variant), | acceptor) | | |
| | c.628G > T | Asp210Tyr | 0 | 0 |
| 21 | c.4229delC | Ala1410GlufsTer24 | 0 | 0 |
| | c.2773C > T | Leu925Phe | 0 | 0 |
| 22 | c.754C > T | Arg252Ter | 0 | 0 |
| | c.2872T > C | Tyr958His | 3.98e−6 | 0 |

[symbol]

indicates data missing or illegible when filed

TABLE 3
Clinical summary of GEMIN5 familes 23-29.

| | Family | | | | | | | | |
|---|---|---|---|---|---|---|---|---|---|
| | 23 | 24 | 25 | 26 | 27 | 28 | | 29 | |
| | Patient number | | | | | | | | |
| | 1 | 2 | 3 | 4 | 5 | 6 | 7 | 8 | 9 |
| | Gender | | | | | | | | |
| | M | M | M | M | M | M | F | M | M |
| [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] |
| Development | | | | | | | | | |
| [symbol] | Y | Y | Y | Y | Y | Y | Y | Y | Y |
| [symbol] | N | N | N | N | N | N | N | N | N |
| [symbol] | Y | Y | Y | Y | Y | Y | Y | N | Y |
| [symbol] | Y | N | Y | Y | Y | Y | Y | Y | Y |
| [symbol] | Y | N | Y | Y | Y | Y | Y | N | N |
| [symbol] | | | | | | | | | |
| [symbol] | [symbol] | Y | Y | Y | Y | Y | Y | Y | Y |
| [symbol] | N | N | Y | N | N | [symbol] | Y | Y | Y |
| [symbol] | Y | N | Y | Y | Y | Y | N | N | N |
| [symbol] | N | N | B | N | B | B | B | B | B |
| [symbol] | Y | Y | Y | Y | Y | Y | Y | Y | Y |
| [symbol] | | | | N | | | | N | [symbol] |
| [symbol] | | | | | | | | | |
| [symbol] | [symbol] | [symbol] | [symbol] | [symbol] | [symbol] | [symbol] | [symbol] | [symbol] | [symbol] |

| | | | | | | | |
|---|---|---|---|---|---|---|---|
| | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] |

[symbol]

indicates data missing or illegible when filed

TABLE 4
Description of GEMIN5 variants in families 23-29.

| | GEMIN5 | GEMIN5 | Allele | |
|---|---|---|---|---|
| | Variant | Variant | frequency | Number of |
| | (cDNA) | (protein) | (hetero- | homo- |
| Family | (NM_015465.5) | (NP_056280.2) | zygous) | zygous |
| 23 | c.T3844C | p.Tyr1282His | 4.01e−6 | 0 |
| | c.C3615G | p.Cys1205Trp | 0 | 0 |
| 24 | c.3041G > A | p.Arg1014Gln | 3.92e−5 | 0 |
| | c.1600-2A > G | c.1600-2A > G | 0 | 0 |
| 25 | c.3026C > T | p.Arg1016Cys | 4.48e−3 | 5 |
| | c.1452dup | p.Met485Hisfs*27 | 0 | 0 |
| 26 | c.3046 C > T | p.Arg1016Cys | 4.48e−3 | 5 |
| | c.2693-2700del | p.Arg899fs*3 | 0 | 0 |
| 27 | c.1119 G > A | p.Trp373* | 0 | 0 |
| | c.3046 C > T | p.Arg1016Cys | 4.48e−3 | 5 |
| 28 | c.2981C > T | p.Ala994Val | 2.39e−5 | 0 |
| 29 | c.1781 C > G | p.Pro594Arg | 2.40e−3 | 6 |
| | c.1624A > g | p.Ser543Gly | 2.51e−4 | 0 |

Furthermore, the brain Magnetic resonance imaging (MRI) in all patients revealed cerebellar atrophy. Patient 4, 5, and 6 (Family 3 and Family 4) had cerebellar atrophy on brain MRI which was performed prior to the age of 6 months, suggesting the possibility of cerebellar hypoplasia. Patient 20 (Family 13), patient 24, and 25 (Family 17) had progressive cerebellar atrophy on repeat imaging. Patient 4 (Family 3), patient 17 (Family 11), patient 20 (family 13), and patients 24 and 25 (Family 17) had a progressive phenotype on clinical evaluation.

In Cohort 1, 30 variants in GEMIN5 were identified, with four of them to be presumed loss-of-function (family 10, 15, 17, and 20), along with 22 missense variants. All variants were evolutionary conserved residues across various species and were rare or absent in gnomAD (Table 2). The missense variants were predicted to be pathogenic and probably damaging in nature by various in silico prediction tools such as Polyphen-2, PROVEAN, SNAP2, muPRO, PhD SNP, and SIFT (Table 5). Eight of the GEMIN5 missense variants are located in conserved alpha helixes in the monomer-monomer interface (p.His1364Pro, p.His923Pro, p.Ile988Phe, p. Ser1000Pro, p. Ala1007Thr, p. Asp1019Glu, p.Leu1367Pro, and p.Leu1119Ser), whereas six missense variants (p. Ser73Pro, p. His162Arg, p.Asp210Tyr, Val611Met, p.Gly683Asp, and p.Asp704Glu) are located in the WD40 domain, and five variants (p.Tyr1282His, p.Tyr1286Cys, p. Tyr1286Asn, p. His1264Pro, and p. Leu1367Pro) are located in the RNA binding site 1 (RBS1). Six of the variants involve a proline substitution (p. Ser73Pro, p.His923Pro, p.Ser1000Pro, p.Leu1068Pro, p.His1364Pro, and p.Leu1367Pro), an amino acid which is well-known for disrupting alpha helix secondary structure.

TABLE 5
In silico variant prediction of GEMIN5 variants in families 1-22.

| | Prediction tools | | | | | |
|---|---|---|---|---|---|---|
| GEMIN5 | PholyPhen- | | | | PhD | |
| Variants | 2 | PROVEAN | SNAP2 | mu PRO | SNP | SIFT |
| p.(Leu1068Pro) | Damaging | Deleterious | Pathogenic | Decreased | Disease | Disease |
| | | (−4.922) | | Stability | Causing | Causing |
| | | | | DDG = −2.21579 | | |
| p.(Ala1007Thr) | Damaging | Deleterious | Neutral | Decreased | Neutral | Disease |
| | | (−3.200) | | Stability | | Causing |
| | | | | DDG = −0.97637 | | |
| p.(His923Pro) | Damaging | Deleterious | Pathogenic | Decreased | Disease | Disease |
| | | (−2.189) | | Stability | Causing | Causing |
| | | | | DDG = −0.77411 | | |
| p.(His913Arg) | Damaging | Deleterious | Pathogenic | Decreased | Disease | Disease |
| | | (−4.778) | | Stability | Causing | Causing |
| | | | | DDG = −0.57662 | | |
| p.(Asp704Glu) | Damaging | Deleterious | Pathogenic | Decreased | Disease | Disease |
| | | (−3.489) | | Stability | Causing | Causing |
| | | | | DDG = −0.57662 | | |
| p.(Gly683Asp) | Damaging | Deleterious | Neutral | Decreased | Disease | Neutral |
| | | (−5.883) | | Stability | Causing | |
| | | | | DDG = −0.65471 | | |
| p.(Ser1000Pro) | Light | Neutral | Neutral | Decreased | Disease | Disease |
| | | (−2.483) | | Stability | Causing | Causing |
| | | | | DDG = −1.2372 | | |

In addition, nine patients (families 22-29) were reported with a spastic ataxia, and cerebellar atrophy. All patients had extensive metabolic and genetic testing, which was unrevealing before NGS revealed biallelic variants in the GEMIN5 gene. All variants were rare with allele frequencies ranging from 0 to 4.48e-3 as a heterozygote and showed an autosomal recessive inheritance pattern (Table 3). The majority of these variants, with two exceptions, (p. Arg1016Cys and p. Pro594Arg), were never reported in the GnomAD database (Table 3). The p. Arg1016Cys was present with the other allele disrupted by a frameshift or termination mutation among three patients from three unrelated families. The p. Arg1016Cys variant is likely to affect the dimerization domain which might result in downstream functional abnormality. Similarly, it was found that the p. Pro594Arg allele with another missense variant in two patients (Table 4). The variants identified were missense, frame shift, termination, and a predicted splice site variant. All missense variants affected were conserved amino acid residues and predicted to be pathogenic by various computational prediction tools (Table 6).

8/9 patients presented in the first 2 years of life with concerns for delayed motor development. No childhood motor or cognitive regression was observed in any of the patients. While all patients had signs of motor dysfunction, the neurological exam ranged from hypotonia and tremulousness to severe spastic ataxia. While central hypotonia was observed in some patients, appendicular tone ranged from normal to spastic. All patients presented with normal to brisk reflexes. All ambulatory patients were assessed by neurologists and were noted to be ataxic on clinical examination. The disease progression was variable among our patients, as some of the patients had a static course, while others showed a possibly slow, progressive ataxia. MRI of the brain showed cerebellar atrophy in all patients.

Stratified by age of onset and severity from both cohorts of patient families with GEMIN5-related disease, the patients fell into 3 main groups based on neurological involvement: (1) Infantile onset severe global developmental delay with cerebellar atrophy on neuroimaging. These patients presented in infancy with severe hypotonia and global developmental delay. Neuroimaging confirmed cerebellar atrophy; (2) Late infantile onset developmental delay and ataxia syndrome with cerebellar atrophy on neuroimaging; or (3) Juvenile/Adult-onset spastic ataxia syndrome with cerebellar atrophy on neuroimaging.

Other clinical features seen in the patients included cataracts, strabismus, and nystagmus. Overall, all variants of GEMIN5 appeared to perturb GEMIN5 protein structure and function(s) and result in deleterious neurological symptoms.

Using in silico tools, it was determined that GEMIN5 variants are deleterious in nature and lead to loss of protein function and stability (Tables 3 and 6). Amino acid substitution at key sites within a protein due to a single nucleotide variant (SNV) may result in various conformation changes, including remodeling or alteration of interaction network, salt bridges, and hydrogen bonds. These changes may perturb the kinetics of protein folding and can cause the destabilization of protein and impairing its subsequent function or interaction with other molecules (Dill et al., Proc Natl Acad Sci USA, 1993, 90 (5): 1942-1946; Teng et al., Int J Comput Biol Drug Des, 2010, 3 (4): 334-349). To determine the effect of GEMIN5 variants on the protein conformation and interaction of surrounding amino acids, PyMOL was used to predict the structural changes caused by five possibly deleterious substitutions-two GEMIN5 variants from Cohort 1 (p. Asp704Glu and p. Gly683Asp) and three GEMIN5 variants from Cohort 2 (p. Ser543Gly, p. Pro594Arg, p. Arg1016Cys). All of the following variants were located within the WD40 repeat domains (PDB ID: 5H1J), except p. Arg1016Cys, which is located in the tetratricopeptide (TPR)-like dimerization domain (PDB ID: 6RNS) of the GEMIN5 protein. By limiting the affected area of mutagenesis to 5 angstroms, changes were measured in the orientation and conformation of surrounding amino acids by calculating their distance from the substituted GEMIN5 variant.

TABLE 2
In silico variant prediction of GEMIN5 variants in families 23-29.

| | | Prediction tool | | | | |
|---|---|---|---|---|---|---|
| | GEMIN5 | PholyPhen- | | | | |
| Family | variant | 2 | PROVEAN | SNAP2 | mu PRO | SIFT |
| 23 | p. Tyr1282His | Damaging | Deleterious | Pathogenic | −1.3449093 | AFFECT |
| | | | | | (DECREASE | PROTEIN |
| | | | | | stability) | function |
| | p. Cys1205Trp | Damaging | Neutral | Pathogenic | −1.6755575 | AFFECT |
| | | | | | (DECREASE | PROTEIN |
| | | | | | stability | function |
| 24 | p. Arg1014Gln | Damaging | Deleterious | Pathogenic | −0.53089832 | AFFECT |
| | | | | | (DECREASE | PROTEIN |
| | | | | | stability) | function |
| | c.1600-2A > G | — | — | — | — | — |
| 25 | p. Arg1016Cys | Damaging | Deleterious | Neutral | −0.96092825 | AFFECT |
| | | | | | (DECREASE | PROTEIN |
| | | | | | stability) | function |
| | p. Met485Hfs*27 | Damaging | Neutral | Neutral | −1.4014936 | AFFECT |
| | | | | | (DECREASE | PROTEIN |
| | | | | | stability) | function |
| 26 | p. Arg1016Cys | Damaging | Deleterious | Neutral | −0.96092825 | AFFECT |
| | | | | | (DECREASE | PROTEIN |
| | | | | | stability) | function |
| | p. Arg899Pfs*3 | Damaging | Deleterious | Pathogenic | −0.59989059 | AFFECT |
| | | | | | (DECREASE | PROTEIN |
| | | | | | stability) | function |
| 27 | p. Trp373* | — | — | — | — | — |
| | p. Arg1016Cys | Damaging | Deleterious | Neutral | −0.96092825 | AFFECT |
| | | | | | (DECREASE | PROTEIN |
| | | | | | stability) | function |
| 28 | p. Ala994Val | Damaging | Deleterious | Neutral | −0.031927059 | AFFECT |
| | | | | | (DECREASE | PROTEIN |
| | | | | | stability) | function |
| 29 | p. Pro594Arg | Damaging | Deleterious | Pathogenic | −0.49351856 | AFFECT |
| | | | | | (DECREASE | PROTEIN |
| | | | | | stability) | function |
| | p. Ser543Gly | Damaging | Deleterious | Pathogenic | −1.6477696 | AFFECT |
| | | | | | (DECREASE | PROTEIN |
| | | | | | stability) | function |

PyMOL structure prediction suggested a possible change in the interaction and angular distance of 704Glu variant with His681, His708, Asp703, Gly683, and Arg682. Likewise, the p. Gly683Asp variant of GEMIN5 which is in a grove between two WD40 domains, induces an ionic charge that may result in the formation of additional bonds with Arg682 and Phe705, as well as a change in the possible conformation due to its interaction with Arg33.

The variant p. Ser543Gly in GEMIN5 may lead to changes in its angular parameters and distance from Ile584, Ser585, and Lys545 respectively, which could result in structural changes in anaphase-promoting complex subunit 4, the WD40 domain (540-588 aa) of GEMIN5. Similarly, it was found that the conversion of Pro594, a non-polar amino acid, to the positively charged arginine might affect its interactions with Glu595, Gln593, and Tyr598. Proline acts as a structural disruptor in alpha and beta helixes which is a prerequisite for protein folding and structure. Therefore, its conversion to arginine, an amino acid found at the active centers of the protein and required for interactions with phosphorylated substrates, might result in the disruption of GEMIN5 structure and its interaction with other proteins.

The p. Arg1016Cys variant of GEMIN5 lies in the TPR-like dimerization domain which modulates the interaction of GEMIN5 with other proteins. Thus, the substitution of the highly polar arginine, which is typically found in the interface of the two proteins, to a small and neutrally charged cysteine, may affect its conformation and dimerization function. PyMOL predicted the possible changes in the distance and bonding with Arg1014, Pro1017, and Asp1019. Furthermore, GEMIN5 variant p. Asp1019Cys has been reported to disrupt the dimerization properties of GEMIN5.

To understand the consequences of biallelic variants in GEMIN5, PBMCs were reprogrammed from the Leu1068Pro/Leu1068Pro patient and an unaffected parent carrying Leu1068Pro/+into induced pluripotent stem cell lines (iPSC). Since the His913Arg patients were not alive, CRISPR/Cas9 was used to engineer the p.His913Arg heterozygote (referred herewith as control) and homozygous variants in a healthy control iPSC line. After doing extensive quality control testing of both iPSC lines, including sequencing and karyotyping analysis, the cells were differentiated into neuronal cells. Two independent isogenic iPSC clones with the homozygous p.His913Arg variant, His913Arg^{A6}, and His913ArgAll were used for the study.

By IF, a drastic decrease in the cytoplasmic distribution of GEMIN5 in the homozygous neuronal cells, p.His913Arg and p.Leu1068Pro, was found while neurons expressing heterozygous variants showed a normal physiological nuclear-cytoplasmic distribution of GEMIN5. In contrast to His913Arg, homozygous Leu1068Pro neurons showed scattered punctate expression of GEMIN5 in the cytoplasm. No aberrant changes were seen in GEMIN5 nuclear levels between the homozygous and control groups. Since GEMIN5 is a component of the SMN complex involved in snRNP spliceosomal assembly (Battle et al., Cold Spring Harb Symp Quant Biol, 2016, 71:313-320; Battle et al. Mol Cell, 2016, 23 (2): 273-279), the mislocalization of mutant GEMIN5 was examined to determine if it had any impact on the sub-cellular distribution pattern of other snRNP complex proteins, such as SMN and GEMIN2. SMN showed no obvious alterations in its distribution pattern between homozygous and control neurons. It was observed that GEMIN2 showed a similar distribution pattern of GEMIN5 in homozygous His913Arg and Leu1068Pro neurons. GEMIN2 levels showed a significant reduction in the cytoplasm with unaltered nuclear levels in homozygous His913Arg and Leu1068Pro neurons compared to controls.

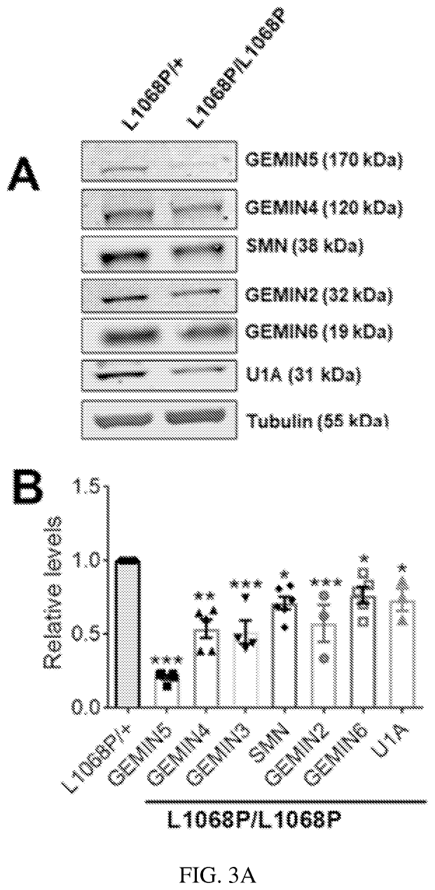

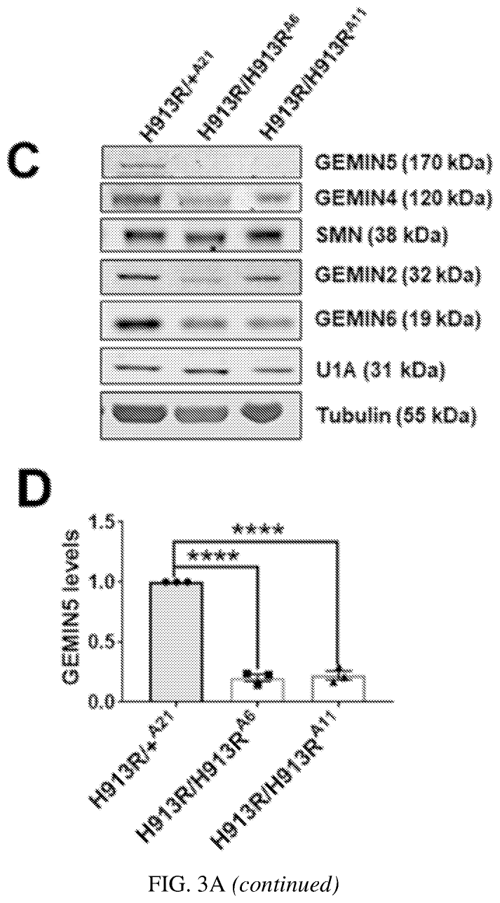

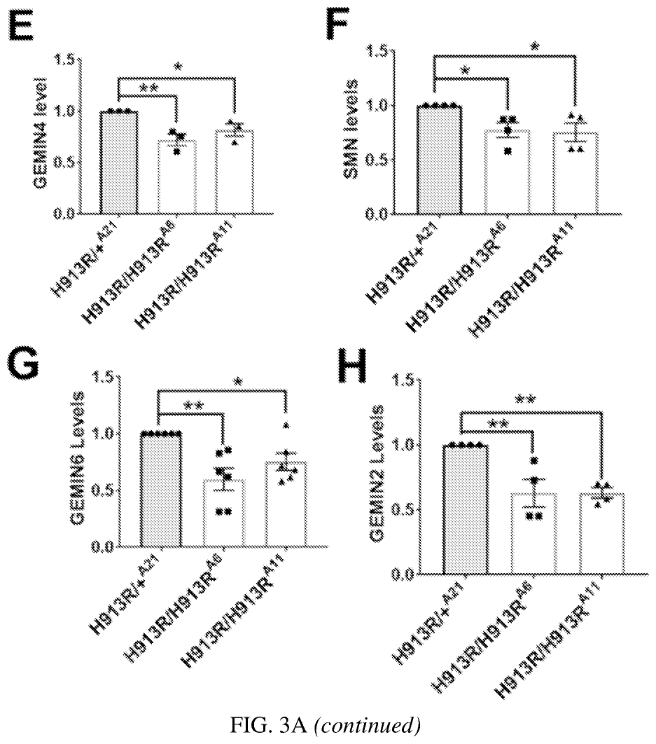

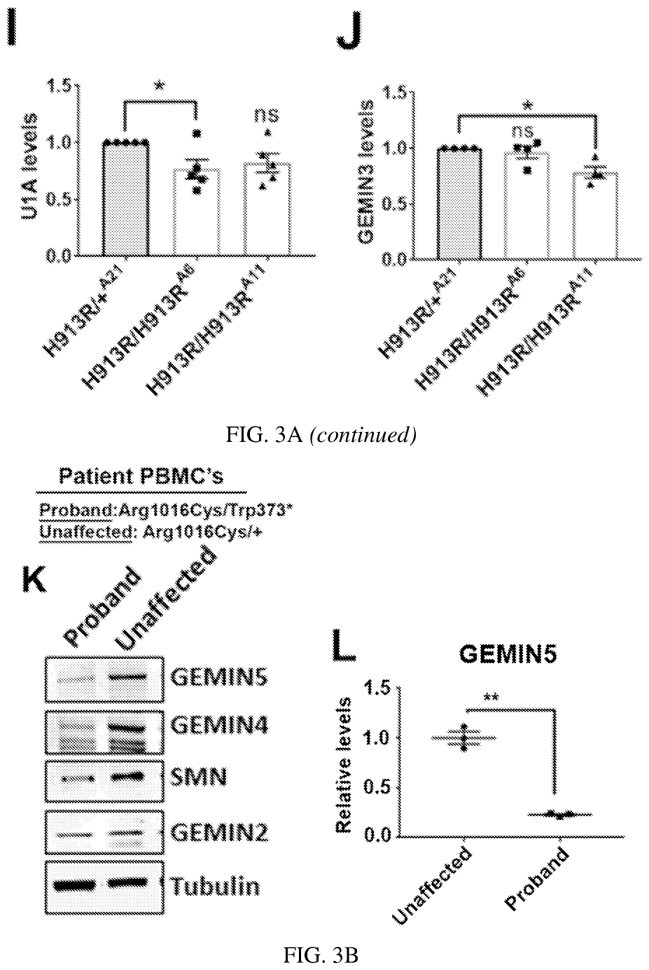

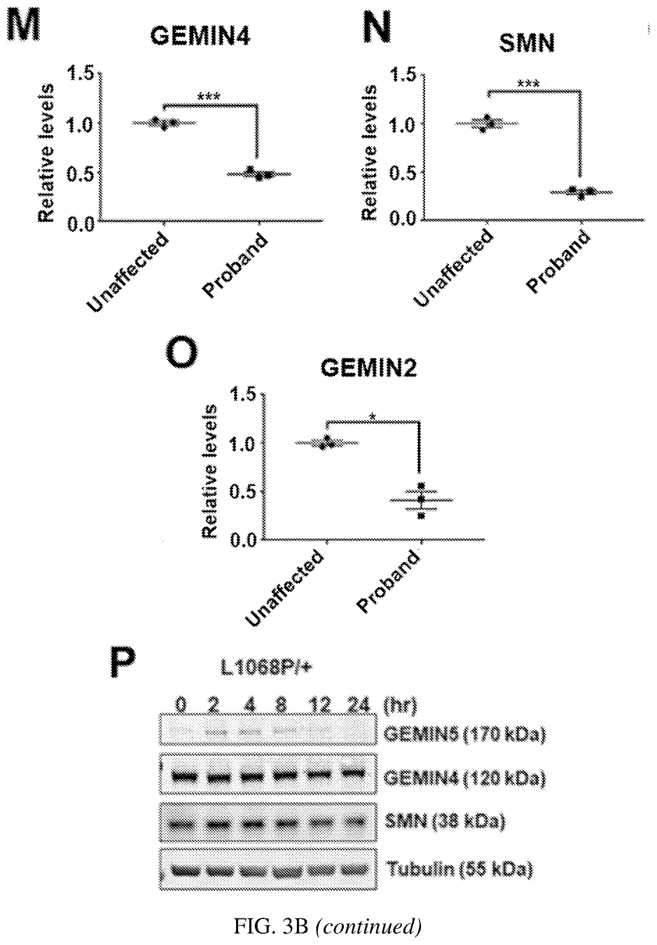

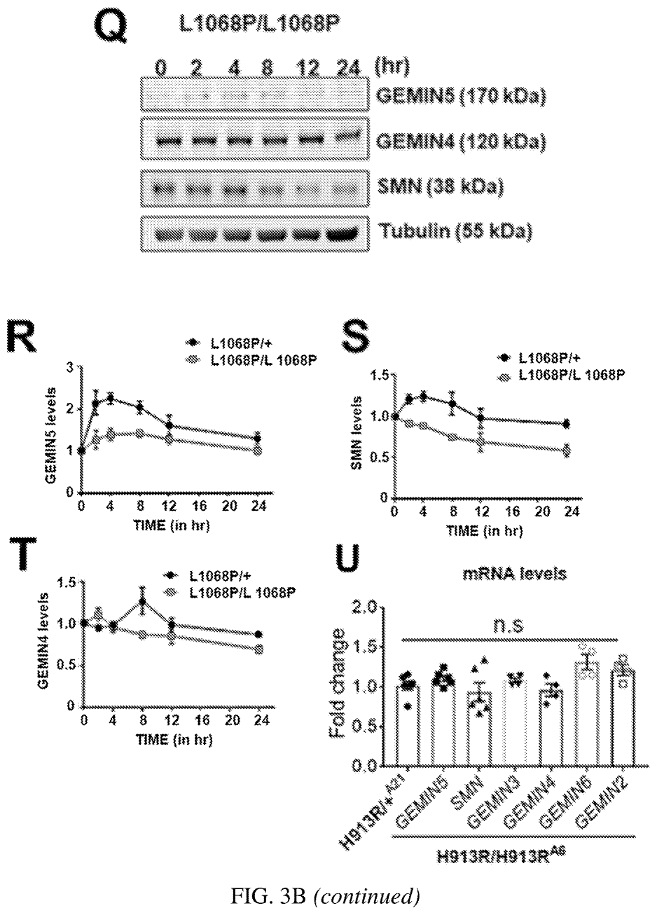

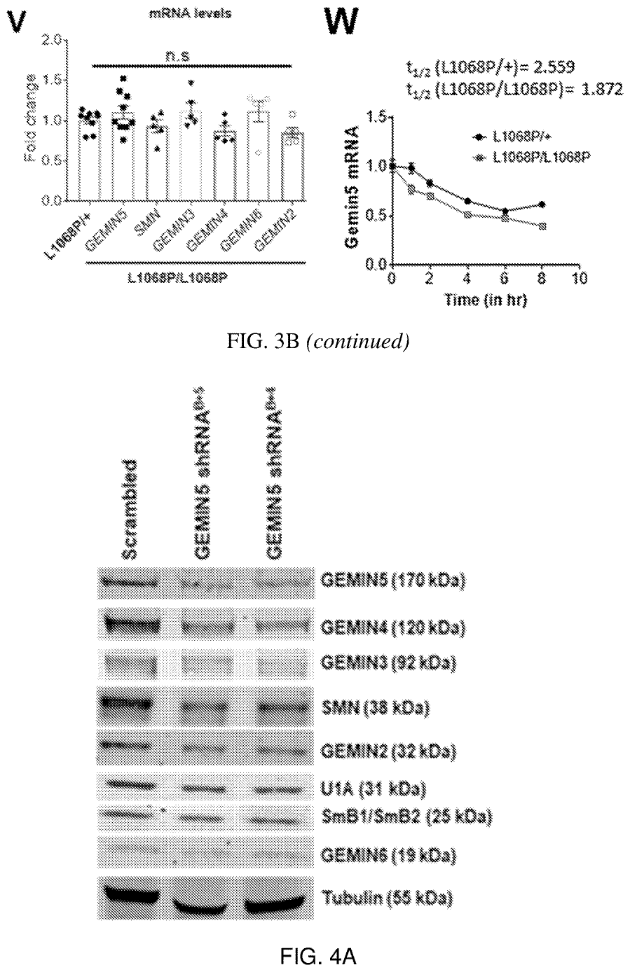

The effect of GEMIN5 variants on the expression levels of GEMIN5, as well as its interacting partners of the SMN complex (Gubitz et al., J Biol Chem, 2002, 277 (7): 5631-5636; Battle et al., 2006), was investigated. The levels of GEMIN5 were drastically reduced by approximately 70-80% in Leu1068Pro and His913Arg patient neurons, as compared to controls (FIG. 3A(A)-(D)). A significant reduction in the protein levels of GEMIN4, GEMIN3, GEMIN2, GEMIN6, SMN, and U1A in Leu1068Pro (FIG. 3A(A)-(B)) and His913Arg (FIG. 3A (C) and (E)-(J)) patient neurons as compared to controls was also observed. In addition, PBMCs were isolated from the Family 27 GEMIN5 patient carrying p. Trp373* and p. Arg1016Cys variants, as well as from the unaffected heterozygous parent (father). Similarly, to the iPSC derived neurons, WB was performed with antibodies to GEMIN5, SMN, GEMIN4, and GEMIN2 to assess protein levels (FIG. 3B(K)). A significant reduction in GEMIN5, SMN, GEMIN4, and GEMIN2 protein levels in the patient sample as compared to the unaffected parent was observed (FIG. 3B(L)-(O)).

To examine the possible underlying mechanisms responsible for the reduced intracellular levels of GEMIN5, GEMIN5's protein stability was compared between the Leu1068Pro patient and control neurons (FIG. 3B(P) and (Q)). WB analysis revealed an initial build-up of GEMIN5 for four hours followed by a gradual drop off in control neurons, whereas an initial reduction in GEMIN5 levels after two hours of CHX treatment in Leu1068Pro patient neurons was observed (FIG. 3B(R)-(T)). Likewise, SMN protein levels showed a steady reduction after two hours of CHX treatment in homozygous Leu1068Pro as compared to heterozygous neurons (FIG. 3B(S)). No obvious changes were seen in GEMIN4 protein stability (FIG. 3B(T)). To address any possible link between reduced GEMIN5 protein levels and its stability with the degradation pattern, the ubiquitination profile of the His913Arg homozygous and control neurons by IF were examined. A robust increase in ubiquitinylated puncta in the cytoplasm and axons of homozygous His913Arg neurons as compared to heterozygotes was observed.

qPCR was performed to determine the basal expression of GEMIN5 mRNAs and found no significant difference in transcript levels between homo- and heterozygous His913Arg and Leu1068Pro neurons (FIG. 3B(U)-(V)). To determine mRNA stability, Leu1068Pro patient neurons were treated with the global transcriptional inhibitor actinomycin D (ActD) for 0, 1, 2, 4, 6, and 8 hours and qPCR was performed on the corresponding total RNAs (FIG. 3B(W)). GEMIN5 mRNAs were found to be significantly less stable in Leu1068Pro homozygous neurons with a half-life (t_{1/2}) of 1.872 in contrast to a t_{1/2} of 2.559 in heterozygotes. Thus, the differential reduction of GEMIN5 in homozygous variants was due to difference in its mRNA and protein stability rather than transcriptional dysregulation.

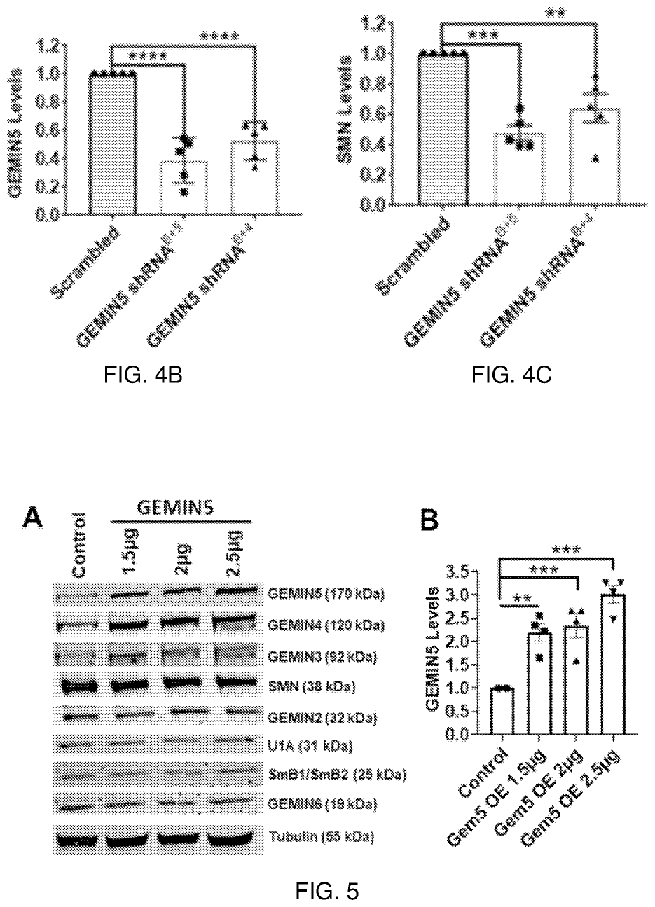

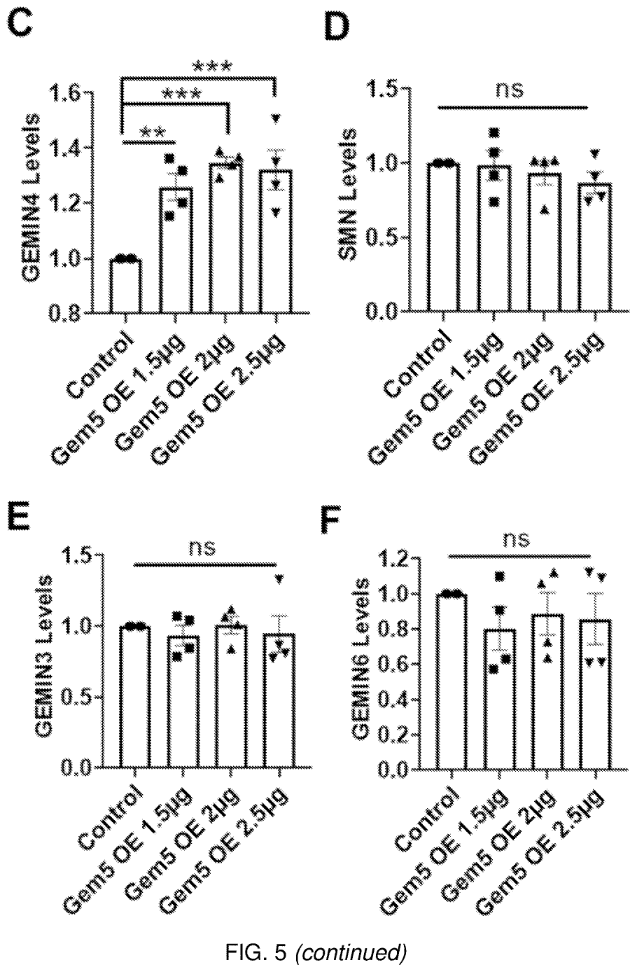

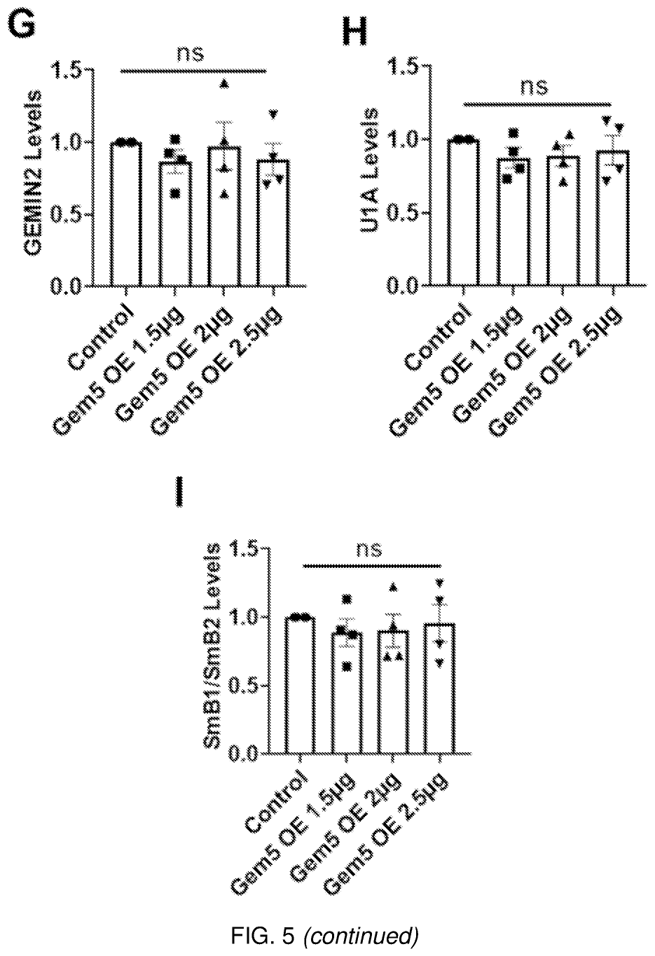

HEK293T cells were transfected with different shRNA constructs against GEMIN5 and measured the protein levels by WB. However, in order to get the robust knockdown (KD) of up to approximately 60-70%, similar to what was seen in homozygous patient iPSC neurons, HEK293T cells were co-transfected with two different combinations of shRNAs with the highest KD efficiency (shRNA B with shRNA 5 and 4) and the levels of SMN complex proteins were evaluated by WB (FIG. 4A). The effect of decreased GEMIN5 on members of the SMN complex was observed to be dosage-dependent, and significantly alleviated levels of SMN, GEMIN4, GEMIN3, GEMIN6, GEMIN2, and SmB1/B2 proteins only when GEMIN5 levels were reduced to below approximately 65% (FIGS. 4B and 4C). Reciprocal studies in HEK cells were completed, where different concentrations of GEMIN5 were overexpressed to determine its subsequent effects on SMN complex proteins. Apart from GEMIN4, no significant changes in the levels of SMN, U1A, SmB1/B2, and other GEM proteins were observed (FIG. 5).

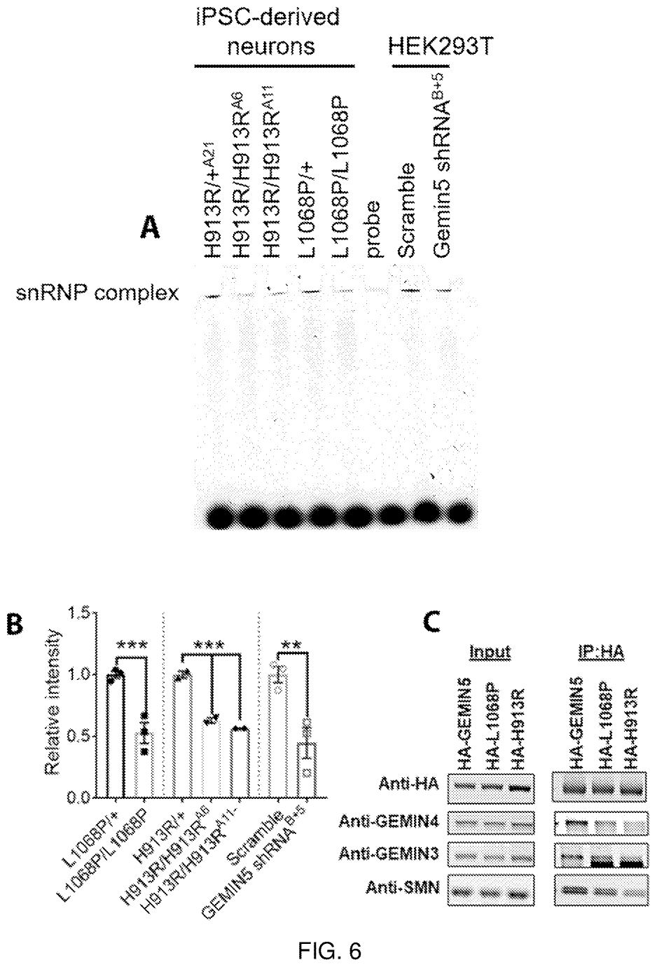

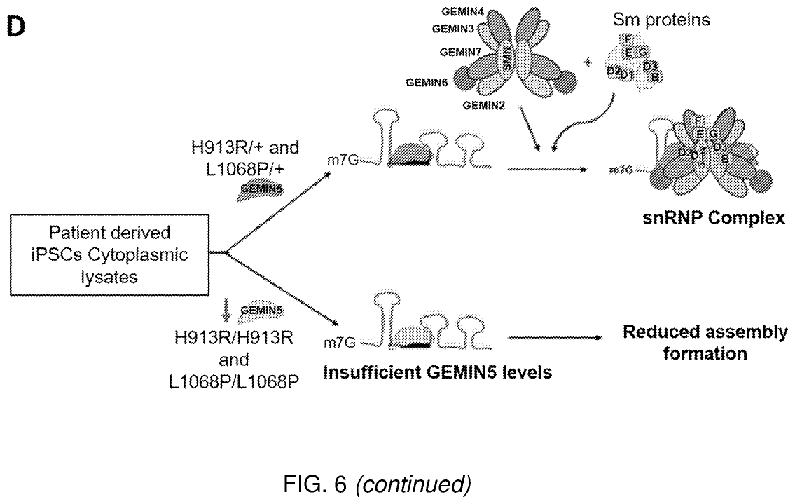

To determine if the pathogenic GEMIN5 variants effected the assembly of core Sm proteins in the SMN-snRNA complex, snRNP assembly was reconstituted by using in vitro-transcribed 3′Cy3-biotinylated-UlsnRNA and cytoplasmic extracts from Leu1068Pro and His913Arg differentiated neurons. To examine the impact of loss of GEMIN5 on the assembly formation, extract from HEK293T cells transfected with or without GEMIN5 shRNA was also used. By native-PAGE, a distinct band representative of SMN-Sm assembly formation in control iPSC neurons and HEK293T control extracts was found. However, the assembly was drastically reduced in extracts from homozygous Leu1068Pro and His913Arg neurons as well as in HEK293T with GEMIN5 shRNA (FIG. 6(A)-(B)), where the loss of GEMIN5 in Leu1068Pro and His913Arg neurons led to disruption of snRNP assembly formation (FIG. 6 (D)). During assembly formation, GEMIN5 interacts with GEMIN3 and GEMIN4 and delivers pre-snRNA to SMN-GEMIN2-Sm protein complex. To assess if the reduced SMN assembly formation is related to the interaction of GEMIN5 variants with other GEM proteins and SMN, immunoprecipitation was performed by using anti-HA beads to affinity purify HA-tagged GEMIN5 WT, Leu1068Pro and His913Arg variants and their interacting proteins in HEK-293T cells. As shown in FIG. 6(C), the His913Arg and Leu1068Pro mutation in GEMIN5 drastically reduced GEMIN5's interaction with SMN, GEMIN4, and GEMIN3 as compared to WT.

GEMIN5 patient neurons show a distinct and unique transcriptomic signature as compared to SMA patient neurons: RNA-sequencing analysis was performed in iPSC-derived differentiated neurons with biallelic mutant GEMIN5 (GEMIN5^{H913R}) and compared this dataset with a published dataset from SMA (SMN1^{Ex7del}) patient iPSC motor neurons. By using this in silico approach, differentially expressed transcripts (DEGs) were identified using a p-value threshold of ≤0.01 adjusted for statistical significance, and a log fold change of ≥1.5. The analysis showed a consequential number of downregulated genes in GEMIN5^{H913R} compared to SMN1^{Ex7del} patient neurons. By comparing the significant DEGs in SMN1^{Ex7del} and GEMIN5^{H913R} patient neurons, 1,278 and 3,004 transcripts unique to GEMIN5^{H913R} and SMN1^{Ex7del}, respectively, were determined, whereas 622 transcripts were shared among these two disease conditions. Heat map comparison with hierarchal clustering of the top 40 common DEGs in SMN1^{Ex7del} and GEMIN5^{H913R} showed a contrasting expression trend. It was observed that a subset of transcripts upregulated in SMN1^{Ex7del} showed an opposite downregulated trend compared to GEMIN5^{H913R} iPSC neurons.

Gene ontology (GO) and Biological Process Ontology (BP) enrichment analysis were performed on the DEGs from both datasets and compared the top 30 identified pathways with adjusted p-value<0.01 & log 2 (fold change)≥1.5 between the two groups. It was found that SMN1^{Ex7del} and GEMIN5^{H913R} shared only five notable pathways involved in the development of the autonomic nervous system, regulation of cell cycle, retinoic acid signaling, and postsynaptic membrane component. However, the majority of pathways altered in GEMIN5^{H913R} were distinct from SMN1^{Ex7del}. The pathways upregulated in GEMIN5^{H913R} are associated with regulation of postsynaptic membrane potential, neurotransmitter secretion, transport, and signaling pathways, whereas the downregulated pathways were linked to regulation of developmental process, extracellular matrix organization, nuclear transport, and signal transduction. The pathways modulated in SMN1^{Ex7del} were notedly involved in nerve development and morphogenesis, intracellular receptor signaling pathways, synaptic membrane adhesion, response to DNA damage, and regulation of ribosomal assembly. Furthermore, the top 40 most significantly expressed DEGs in both GEMIN5^{H913R} and SMN1^{Ex7del} showed little overlap in their expression patterns. The GEMIN5 RNA sequencing data was validated by performing qPCR on three highly upregulated (SOX14, GBX2, and PDZRN4) and three highly downregulated (LRRC1, NXX2.1, and STX11) genes. The transcriptomic comparison between SMN1^{Ex7del} and GEMIN5^{H913R} patient neurons showed that mutations in GEMIN5 disrupted distinctive developmental and neurological pathways with slight overlap with SMA.

The global splicing defects in GEMIN5^{H913R} homozygous neurons compared to controls was investigated. Differential splicing analysis was performed based on isoform expression by adjusting the threshold value to 5% and found 99 differentially spliced genes (DSGs) with a total of 440 isoforms in GEMIN5^{H913R} compared to controls. Functional enrichment analysis of the differentially spliced genes (DSGs) with the FDR adjusted to <0.05 showed that the majority of the DSGs were involved in alternative splicing, phosphoprotein function, and cytoplasmic function in GEMIN5^{H913R} compared to controls.

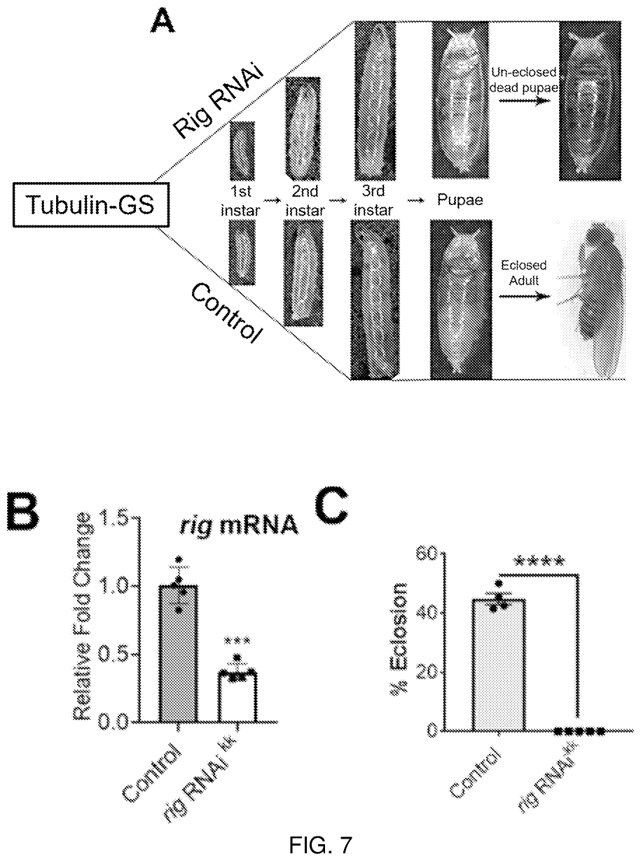

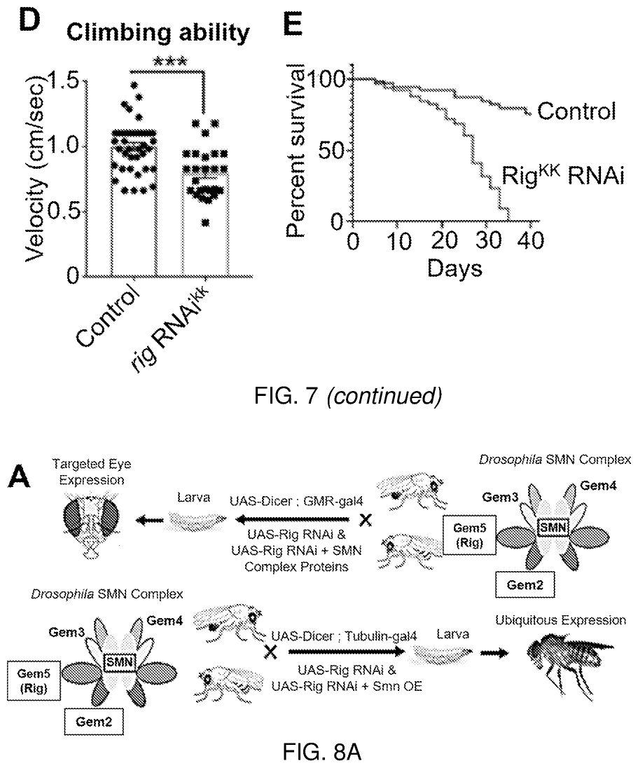

The possible consequences of the loss of GEMIN5 was investigated in an in vivo Drosophila model. The clinical manifestations related to GEMIN5 variants occur at very early stages in humans. Therefore, the loss of Rigor mortis (Rig), a fly orthologue of human GEMIN5, by RNAi mediated knockdown was studied to determine any impact on the development of flies. RNAi transgene against Rigor mortis was expressed in flies by using the inducible tubulin-GAL4/upstream activation sequence (UAS) system and monitored the development of flies from egg to adults on 1 mM RU486 drug food (FIG. 7(A)). Complete pupal lethality in the Rig RNAi expressing flies as compared to EGFP-controls was found (FIG. 7(C)), due to severe late-developmental defects with 60% loss of Rig as validated by qPCR (FIG. 7(B)). Control and Rig KD animals were stained with the pre-synaptic marker, horse radish peroxidase (HRP), to assess the NMJs. A significant reduction in the bouton size of larvae with Rig KD was found, as compared to the EGFP-controls. To examine further motor function defects, rapid iterative negative geotaxis (RING) assay was performed on neuronally expressing Rig RNAi lines (FIG. 7(D). It was found that Rig KD significantly reduced the climbing ability of adult flies compared to control animals. Three patients with biallelic GEMIN5 variants showed early lethality and the loss of GEMIN5 protein was identified in the homozygous patient derived IPSCs neurons. Therefore, the effect of the loss of GEMIN5 protein on the life span of adult flies was investigated. Flies expressing Rig KD (n=103) were monitored over the span of 45 days and 100% mortality in Rig KD flies was found after 33 days, as compared to 19% in w1118 controls (FIG. 7(E)). The loss of Rigor mortis was determined to lead to premature lethality, motor dysfunctions, and reduced life span in vivo, which replicates the neurological symptoms found in GEMIN5 patients.

To further understand the consequences of loss of function Gemin5 in vivo, Gemin5 knockout (GEMIN5-DEL558) mice were created through the International Mouse Phenotyping Consortium (Dickinson et al., 2016, Nature, 537 (7621): 508-514; Mianne et al., Methods, 2017, 121-122:68-76). CRISPR/Cas9 was utilized to delete 558 nucleotides including exon 7 of the Mus Musculus Gemin5 gene. This deletion of 558 nucleotides resulted in a frameshift and formation of a premature stop codon in the Gemin5 gene. Heterozygous mice were validated for harboring 1 copy of Gemin5 WT and 1 copy Gemin5 knockout by SR-PCR genotyping. Heterozygous knockout mice were crossed together to assess the percentage of homozygous pups. No postnatal homozygous pups were observed at the PO stage. 63% of the PO pups were heterozygous for the Gemin5 knockout allele, while 37% of pups harbored WT Gemin5 (n=57).

To assess if pups were viable during embryonic development, mice were genotyped at embryonic day E9.5-10.5 across 5 separate litters. No homozygous embryos were observed at E9.5-10.5 (n=30). In addition, the body weight of heterozygous Gemin5 knockout mice were assessed over the course of 20 weeks. There was no significant change in the weight of heterozygous mice compared to wild type control mice (n=15 heterozygote mice, 1,013 control mice). The homozygous loss of Gemin5 was determined to result in early embryonic lethality, while harboring 1 copy of defective Gemin5 did not appear to be detrimental and cause any obvious motor defects.

The CRISPR/Cas9 system was used to generate mutant L1068P and H913R mouse models. GEMIN5 is highly conserved from humans to mice and contains all of the similar protein domains required for GEMIN5 protein function. Highly conserved residues of the mouse Gemin5 gene were selected and introduced the patient mutations L1068P (L1067P in the mouse) and H913R (H912R in the mouse) via CRISPR/Cas9. These two mutations were selected because the His913Arg mutation caused severe neurological phenotypes leading to death within 2 months of age and the Leu1068Pro mutation led to a modest phenotype patients. Heterozygous mice were validated at embryonic stage E3.5 for harboring 1 copy of Gemin5 WT and 1 copy of mutant (H912R or L1067P) by PCR genotyping. Heterozygous knockout mice were crossed together to assess the percentage of homozygous pups. No postnatal homozygous mutant pups were observed at the PO stage. 58% of the PO pups were heterozygous for the mutant Gemin5 H912R allele, while 42% of pups harbored WT Gemin5 (n=45). These findings determined that two copies of Gemin5 were essential for development, as the bi-allelic human GEMIN5 mutations, L1068P and H913R, result in early embryonic lethality in mice.

GEMIN2 protein expression levels were observed to be also reduced along with GEMIN5 in patient neurons as well as in cells with shRNA mediated GEMIN5 knockdown compared to controls (FIGS. 3A, 3B, 4A, 4B, and 4C). Besides SMN and GEMIN5, GEMIN2 is an essential core component required for the assembly of the SMN complex. GEMIN2 binds to SMN and Sm heptameric rings to facilitate their interaction with GEMIN5-snRNA (Ogawa et al., J Biol Chem, 2007, 282 (15): 11122-11134; Zhang et al., Cell, 2011, 146 (3): 384-395).

### Example 2—SMN Regulates GEMIN5 Expression and Acts as a Modifier of GEMIN5-mediated Neurodegeneration

SMN was identified as a genetic modifier of GEMIN5-mediated neurodegeneration in vivo and as a regulator of GEMIN5 expression in mammalian cells and iPSC neurons. Genetic upregulation of SMN via ectopic expression and administration of the SMN2 FDA-approved antisense oligonucleotide, Nusinersen, increased the physiological levels of GEMIN5 in mammalian cells. In addition, a reduction in SMN expression in SMA motor neurons revealed a notable decrease in GEMIN5 protein. Upregulation of SMN via lentiviral expression and Nusinersen administration significantly increased GEMIN5 expression in loss-of-function GEMIN5 patient iPSCs. The Tudor domain of SMN was required for GEMIN5 interaction and regulation. Ectopic expression of SMN rescued the snRNP biogenesis defects exhibited in mutant GEMIN5 iPSC derived neurons as well as loss-of-function GEMIN5 in vivo. Lentiviral SMN expression was shown to rescue the inclusion of alternatively spliced isoforms identified in GEMIN5 iPSC neurons. Therefore, SMN was determined to be a strong regulator of GEMIN5-mediated neurodegeneration and GEMIN5 expression.

### Materials and Methods

Drosophila lines: The fly lines used for this study and motor function assay can be found in Table 7. All Drosophila stocks were cultured on standard media on a 12-hour light/dark cycle.

TABLE 7
Drosophila lines

| Strains | Source | Reference |
|---|---|---|
| UAS-Luciferase | Bloomington Stock Center | Stock #35788 |
| UAS-Dicer | | Dietzl et al., 2007 |
| UAS-Rig RNAi | Bloomington Stock Center | VDRC 105403 |
| UAS-3xHA-Gem2 | FlyORF | Fly Line ID F003047 |
| UAS-Flag-SMN | Gift from Dr. Spyros | Chang et al., 2008 |
| | Artavanis-Tsakonas | |
| UAS-Smn RNAi | RNAi Bloomington stock | Stock #36621 |
| | center | |
| GMR-Gal4 | Bloomington Stock Center | #1104 Dietzl et al., |
| | | 2007 |
| Tubulin-gal4 | Gift from Dr. Scott Pletcher | Dietzl et al., 2007 |

Genetic Crosses: The Gal4/UAS system was used for targeted misexpression of the inducible transgene (Brand et al., Development, 1993, 118 (2): 401-415). All Gal4/UAS crosses were maintained at 25° C. and 29° C. to sample different induction levels of transgenes (Singh et al., Genetics, 2005, 171 (1): 169-183; Raj et al., Sci Rep, 2020, 10 (1): 17221) UAS-dicer/CyO; tub-Gal4/TM3 and UAS-dicer/CyO; GMR-Gal4/Tb stocks were generated to enhance inducible RNAi potency (Dietzl et al., Nature, 2007, 448 (7150): 151-156) in UAS-rig^{KK RNAi}, or UAS-SMN alone (FLAG tagged) or both. The GMR-Gal4 drives expression in the differentiating retinal precursor cells (Moses et al. Genes Dev, 1991, 5 (4): 583-593), whereas tub-GAL4 drives expression ubiquitously. The adults were assessed for phenotypes and frequency analysis was performed. Images of the left eyes of F1 generation, adult female Drosophila were taken at day 1 (or as indicated for ageing experiments) using a Leica M205C dissection microscope equipped with a Leica DFC450 camera. External eye degeneration was quantified using a previously published scoring system (Lanson et al., Hum Mol Genet, 2011, 20 (13): 2510-2523). Eyes were examined for the presence or absence of the following features: supernumerary inter-ommatidial bristles, abnormal bristle orientation, ommatidial fusion, ommatidial pitting, disorganization of ommatidial array, and retinal collapse. If the following features were present, 1 point was given. Two points were added if the affected area involved more than 5% of the eye and 4 points were added if the affected area involved more than 50% of the eye. Comparisons between genotypes were made using Student's t-test assuming equal variances. Eye size was measured using ImageJ software (NIH). Adult fly images were captured at 5× by placing the flies sideways on a clean slide. The images were taken on a MrC5 color camera mounted on an Axioimager.Z1 Zeiss Apotome using a Z-sectioning function of Axiovision software 4.6.3 (Irwin et al., Front Cell Dev Biol, 2020, 8:117; Raj et al., 2020). The final images were prepared using Adobe Photoshop CS6 software.

Larval preparations and immunohistochemistry: Third instar larvae were dissected, fixed, and immunostained as previously described (Anderson et al., Hum Mol Genet, 2018, 27(8): 1366-1381). Animals were dissected in ice-cold PBS (Lonza, #17-516 F), fixed in 4% formaldehyde, washed three times in PBS, incubated in 5% Triton X-100/PBS for 20 min, washed three times in 0.1% PBST (0.1% Triton X-100/PBS), and incubated overnight with primary antibodies: Drosophila coilin (from Dr. Joseph Gall, Carnegie Institution for Science, Baltimore, MD; 1:100) and Drosophila SmB (Y12, Novusbio, 1:100). Larvae were washed three times in 0.1% PBST and incubated with secondary antibodies: anti-rabbit Alexa Flour 568, 1:100 (Invitrogen, #651727); anti-mouse Alexa Flour 647, 1:100 (Invitrogen, #28181). Stained larvae were mounted using DAPI Fluoroshield (Sigma-Aldrich, #F6182). Images were collected on a Nikon A1 eclipse Ti confocal microscope.

Plasmids: The lentiviral vectors used to express EGFP (pLV [Exp]-CMV>EGFP, VB900088-2229upx) and SMN1 (pLV [Exp]-EGFP-CMV>hSMN1, VB201023-1249tdf) in mammalian cells were constructed and packaged by VectorBuilder. The following plasmids were constructed and packaged by VectorBuilder: HA-GEMIN5-WT (VB210409-1232kze), HA-WD40 (VB210819-1201kfk), HA-ΔRBS (VB210819-1225rn), HA-ΔWD40 (VB211122-1218exw), HA-SMN (VB220512-1296yke), HA-SMN Ex1-5 (VB220512-1302gqd), HA-SMN ΔEx3 (VB220512-1299vvx), HA-SMN ΔΕx5 (VB220512-1301jan).

Cell culture and differentiation of iPSC into neuronal cells: HEK293T Cells: HEK cells (ATCC® CRL-3216™) were cultured in Advanced DMEM supplemented with 10% FBS and 1% Glutamax and grown at 37° C. and 5% C_{0.2}.

SMA patient motor neurons: HUVEC iPSC line obtained from the Kurian Lab (Panopoulos et al., PLoS One, 2011, 6 (5): e19743). WTC11 line was generated by the Conklin Lab and purchased from Erasmus MC iPS Core Facility. CS13iSMA was purchased from Cedars Sinai iPSC Core Facility. HGK1, HGK4a, HGK13, HGK22, HGK27 were generated by Dr. Ludwig Heesen and iPIERIAN (Heesen et al., Cell Mol Life Sci, 2016, 73 (10): 2089-2104; Garbes et al., Hum Mol Genet, 2013, 22 (2): 398-407). A description of the SMA patient lines can be found in Table 8. Motor neuron differentiation was performed as described in Example 1, with some modifications (Guo et al. Nat Commun, 2017, 8 (1): 861). To generate the clumps for embryoid body formation, similar size squares were scratched on a ~90% confluent well, treated with collagenase type IV and resuspended in Essential™6 medium (A1516401, Gibco™) on ultra-low attachment flask (Corning). The first two days of the differentiation, neuronal basic medium (DMEM/F12 and Neurobasal supplemented with N₂ and B27 without vitamin A) was supplemented with 3 μM CHIR99021 (4423, Tocris Bioscience), 0.2 μM LDN-193189 (S2618, Selleckchem), 40 μg SB431542 (1614, Tocris Bioscience), and 5 μM Y-27632 (S1049, Selleckchem). From day 3 on, neuronal media was supplemented with 0.1 μM retinoic acid (Sigma) and 500 nM SAG (Merck Millipore). From day 8 on until the end of the differentiation BDNF (10 ng/ml, Peprotech) and GDNF (10 ng/ml, Peprotech) were added to the media. From day 9 to 16, neuronal media was supplemented with 20 μM DAPT (2634, Tocris Bioscience). On day 11, clumps with motor neuron progenitors were dissociated into single cells for plating on 20 μg/ml laminin coated wells. From day 17 on, maturation media containing 10 ng/ml of BDNF, GDNF and CNTF (Peprotech) was added. Every other day media was changed by replacing half of the medium.

TABLE 8
List of iPSC lines

| | Phenotype | SMN1/SMN2 copies |
|---|---|---|
| SMA hiPSC line | | |
| Huvec | Healthy control | 2/2 |
| WTC11 | Healthy control | n.d |
| HGK1 | SMA I | 0/2 |
| HGK4 | SMA II | 0/3 |
| CS13iSMA | SMA II | 0/3 |
| HGK13 | SMA III | 0/3 |
| HGK22 | SMA IIIb | 0/4 |
| HGK27 | SMA IIIb | 0/4 |
| GEMIN5 hiPSC line | | |
| L1068P/+ | Healthy control | 2/2 |
| L1068P/L1068P | GEMIN5-mediated disease | 2/2 |
| H913R/+ | Healthy control | n.d. |
| H913R/H913R | GEMIN5-mediated disease | n.d. |

GEMIN5 iPSC derived neurons: Two different mutant GEMIN5 iPSC lines harboring L1068P and H913R variants were used, as characterized in Kour et al. (Nat Commun, 2021, 12 (1): 2558). PBMCs were reprogrammed from a homozygous GEMIN5 L1068P/L1068P patient (referred to as GEMIN5^{L1068P}) and an unaffected parent (L1068P/+, referred to as control) into induced pluripotent stem cell lines (iPSC). In addition, CRISPR/Cas9 was used to engineer an additional mutant GEMIN5 cell line, GEMIN5 H913R (H913R/+, referred to as control) and homozygous variant GEMIN5 H913R/H913R (referred to as GEMIN5^{H913R}) in a healthy isogenic iPSC line. The iPSCs were cultured and maintained as described in Example 1.

Lentiviral production and transduction: Lentiviral transfer vectors encoding EGFP and SMN were co-transfected with Lenti packaging plasmids (OriGene) into HEK cells using the Turbofectin transfection reagent (OriGene) according to manufacturer's instructions. Following an initial media change, lentiviral supernatant was collected at 24 and 48 hours post-transfection prior to filtration and overnight incubation at 4° C. with 1× Lenti Concentrator Solution (OriGene). The Lenti Particles were then centrifuged at 4,000×g for 120 minutes at 4° C. The resulting pellet was recentrifuged at 4,000×g for 5 min prior to re-suspension in ice-cold, sterile PBS. Pellets were then allowed to dissolve for 1-2 days at 4° C. Resuspended lentiviral particles were then aliquoted. Neuron transductions were performed by diluting lentiviral particles at an MOI of 5 in neuronal differentiation media. Media changes were performed after 48 hours of incubation and all experiments were initiated at 120+hours post-transduction.

Transfections and ASOs: HEK293T cells were transiently transfected with plasmids using Lipofectamine 3000 (Invitrogen L3000001) and used 48 hours after transfection. All ASOs used in this study were obtained from Dharmacon (Lafayette, CO, USA) and encompassed phosphorothioate backbone and 2′OMe modifications throughout the entire sequence. HEK293T cells were transiently transfected with control 10′mer and ASO N1 ASO's at 25, 50, and 100 nM for 48 hours. iPSC neurons were transiently transfected with control 10′mer and ASO N1 ASO's at 500 nM for 72 hours. ASO sequences are as follows: ASO N1: 5′-AUUCACUUUCAUAAUGCUGG-3′ (SEQ ID NO: 32), Control: 5′-UUGCCUUUCU-3′ (SEQ ID NO: 33).

Immunofluorescence: The HEK293T cells and neurons were fixed in 4% paraformaldehyde (PFA) for 15 min and blocked in 0.1% Triton-X in PBS and 5% normal goat serum for 1 hour. The cells were treated overnight with the following antibodies: rabbit anti-GEMIN5 (Millipore Sigma HPA037393, 1:1,000), mouse anti-Smb (Y12) (NovusBio, 1:500), mouse anti-SMN (BD transduction 610646, 1:1,000), mouse anti-coilin (Santa Cruz sc-55594, 1:500), chicken anti-beta-III Tubulin (NOVUS Biologicals NB100-1612-1:1,000), and goat anti-MAP2 (Synaptic System-188 004, 1:1,000). Alexa fluor-488,-568 and 647 secondary antibodies were used from Invitrogen. The cells were mounted using fluoroshield™ with DAPI (Sigma) and images were taken at 60× using Nikon A1-T216.3 confocal microscope.

Western Blot (WB) analysis: Fly heads were collected from each cross and snap-frozen on dry ice. Heads were crushed on dry ice and incubated in RIPA buffer (150 mM sodium chloride, 1% NP40, 0.1% SDS, 1% sodium deoxycholate, 50 mM NaF, 2 mM EDTA, 2 mM DTT, 0.2 mM Na orthovanadate, 1× Roche protease inhibitor #11836170001). Lysates were sonicated and centrifuged to remove debris. Supernatants were boiled in Laemmli buffer (Boston Bioproducts, #BP-111R) for 5 min and loaded onto 4%-12% Nupage Bis-Tris gels (Novex/Life Technologies). Proteins were transferred using the iBlot2 (Life Technologies, #13120134) onto nitrocellulose (iBlot 2 NC regular Stacks, Invitrogen, #IB23001). Western blots were blocked with 2.5% milk solution (BLOT-QuickBlocker reagent, EMB Millipore, #WB57-175GM) and incubated with primary antibody overnight: rabbit Drosophila Rig, 1-1:000 (developed by Bio Boster Biological Technology Co. Ltd, Wuhan, China), rabbit Drosophila Smn (developed by Bio Boster Biological Technology Co. Ltd, Wuhan, China), and mouse anti-tubulin, 1:10,000 (66031, Protein Tech). Blots were washed and incubated in secondary antibody for one hour and imaged on a Licor imager (Odyssey CLx). All western blots were run in triplicate using biological replicates. Protein levels were quantified using Image J software, and statistical analysis was performed with GraphPad Prism six software.

Western Blot for the HEK293T cells and differentiated neurons was completed as described in Example 1, except that the blots were blocked in 2.5% QuickBlocker reagent (EMB Millipore WB57-175GM) and probed overnight with the following antibodies: mouse anti-tubulin (SIGMA, 1:10,000), anti-beta-actin HRP-conjugated (1:10000, HRP-60008, Proteintech), anti-GEMIN5 (Protein Tech p-7210531, 1:1,000), mouse anti-GEMIN2 [2E17](1:2,000), mouse anti-GEMIN4 [F-2](Santa Cruz sc-166418, 1:1,000), and mouse anti-SMN (1:3000, 610646, BD Biosciences). Secondary antibodies used were anti-rabbit DYLight 800 and anti-mouse 680 (Invitrogen, 1:10,000). The blots were imaged using Licor imager (Odyssey CLx). All the blots were run in triplicates and the integrated band densities were calculated using image studio software (Licor).

RT-PCR and qPT-PCR: Total RNA was isolated using the PureLink™ RNA mini kit (Invitrogen). RNA quantity and purity (260/280 and 260/230 ratios) were determined using a NanoDrop ND-1000 spectrophotometer. The iScript Select cDNA Synthesis Kit (BioRad) was then used to produce cDNA from 500 ng of RNA from each sample. For PCR reactions, the cDNA was synthesized in 20 μL reactions using PCR supermix (Invitrogen) following the manufacturer's instructions. PCR products were separated on 6% TBE gels (Invitrogen) and visualized by ethidium bromide staining. Analysis and quantifications of SMN2 splice products were performed using ImageJ software. The primers for Gemin5, Gemin4, Gemin2, Gemin6, SMN1, SMN2-FL, SMN2-Ex7, GAPDH, Rigor Mortis, DmTubulin, ALB exon 12, SMN1 exon 7, SMN2 exon7, ALB+1536F, ALB+1595R, SMN1-Ex7-116F, and SMN1-Ex7-261R, used in the PCR reactions are provided in priority application, U.S. Provisional Patent Application No. 63/477,022 filed Dec. 23, 2023, which is hereby incorporated by reference in its entirety.

For qRT-PCR, all cDNA samples were run on 96-well plates (Applied Biosystems; 4306737) on a 7300 Real-Time PCR System (Applied Biosystems) using the Bio-Rad iQ Supermix (170-8862). Cycle threshold (CT) values were recorded and analyzed following the comparative (CT) method using Prism 6 (GraphPad Software) for statistical analyses. All primers for qPCR were designed PrimerQuest primer design tool (Integrated DNA Technologies). The Primers were designed with the PrimerTime qPCR Assay tool (Integrated DNA Technologies). IDT PrimeTime qPCR Assays were used as the primer/probe solutions. The primers and probes for dm-CTNNB1, dm-DDX17, dm-FBN2, dm-LRP1, dm-NUP210, and dm-STOML2, used in the PCR reactions are provided in priority application, U.S. Provisional Patent Application No. 63/477,022 filed Dec. 23, 2023, which is hereby incorporated by reference in its entirety.

Simplex real-time PCR SMN copy number: Simplex real-time PCR analysis was performed as previously described with some modifications (Passon et al., Genet Test Mol Biomarkers, 2009, 13 (1), 37-42; Stabley et al., Mol Genet Genomic Med, 2015, 3 (4), 248-257). Human serum albumin (ALB) was employed as an endogenous reference gene. Primers and TaqMan MGB 50-labeled VIC probes for the SMN1 and SMN2 exon 7 locus used in this study were as previously described (Anhuf et al, Hum Mutat, 2003, 22 (1): 74-78; Passon et al, 2009). Primers and TaqMan MGB 50-labeled FAM probe for the reference gene Albumin map are in exon 12 and have been designed by using Integrated DNA technology software. All primers and probes for the copy number assay are in appendix table 5. Sample DNA was diluted to the final theoretical concentration of 5.0 ng/mL. Simplex PCR was carried out in quadruplicate for each sample. Each reaction was performed in a total volume of 20 μL containing 10.0 ng of genomic DNA, 10 μL iQ multiplex powermix (Bio-Rad), 300 nM of the gene-specific primers, and 100 nM of the gene specific MGB probe. PCR conditions were 3 min 95° C. and 40 cycles consisting of 15 sec 95° C., and 1 min 60° C. The copy number of SMN1 or SMN2 was calculated using the following equation: SMN1 (SMN2) copy number=raw SMN1 (SMN2) CT value/(raw albumin CT value/2).

RNA sequencing and Alternative splicing (AS) analysis: RNA was isolated from iPSC-derived differentiated neurons from mutant GEMIN5^{H913R} iPSC neurons with EGFP and EGFP-SMN lentiviral expression using the PureLink™ RNA mini kit (Invitrogen). RNA from all samples was extracted, assessed for quality control, and sequenced via paired-end 2×150 bases at approximately 60 million paired-end reads per sample on an Illumina HiSeq platform. RNA library preparations and RNA sequencing reactions were conducted at GENEWIZ, LLC. For consistency, gene expression analysis was performed first for all samples by mapping to the genome (Ensembl Version GRCh38.96) using the STAR aligner. Principal Components Analysis (PCA) confirmed that the samples were sequentially ordered in gene expression space, as expected. Alternative splicing analysis was conducted using rMATS (V4.1.1) with Ensembl gene models (Shen et al., Proc Natl Acad Sci USA, 2014, 111 (51): E5593-E5601). Initially, the categories of alternative splicing events such as skipped exon (SE), mutually exclusive exon, alternative 5′ splice site, alternative 3′ splice site, and retained intron were identified and the schematics were plotted. Further, mapped BAM files were used as an input to detect differences in specific regions of transcripts from a particular gene based on the percentage of splicing inclusion and detect the differential splicing between mutant GEMIN5^{H913R} neurons with and without SMN expression. Output from the analysis included both the reads mapped to splice junctions as well as reads mapped to both splice junctions and exon body. To gain a systematic view of the alternative splicing, the PSI values for these events were retrieved in and created sashimi plots. Sashimi plots of select representative genes as determined by rMATS analysis was plotted using rmats2sashimiplot wrapper by calculating the average inclusion level, average read depth, and the average number of junction-spanning reads of each group.

Statistical analysis: Statistical analyses were done on GraphPad Prism using one-way ANOVA (analysis of variance) followed by a Bonferroni or Tukey post hoc test for comparison between two or more groups.

### Results and Discussion

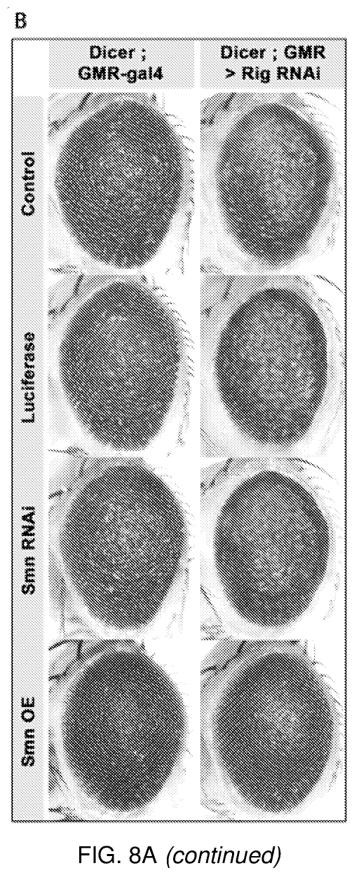

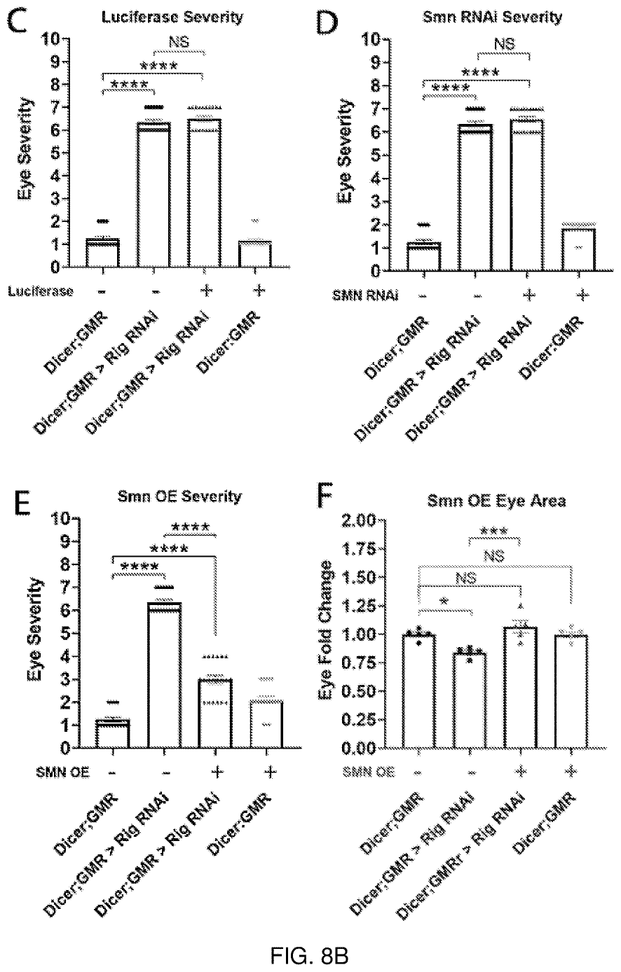

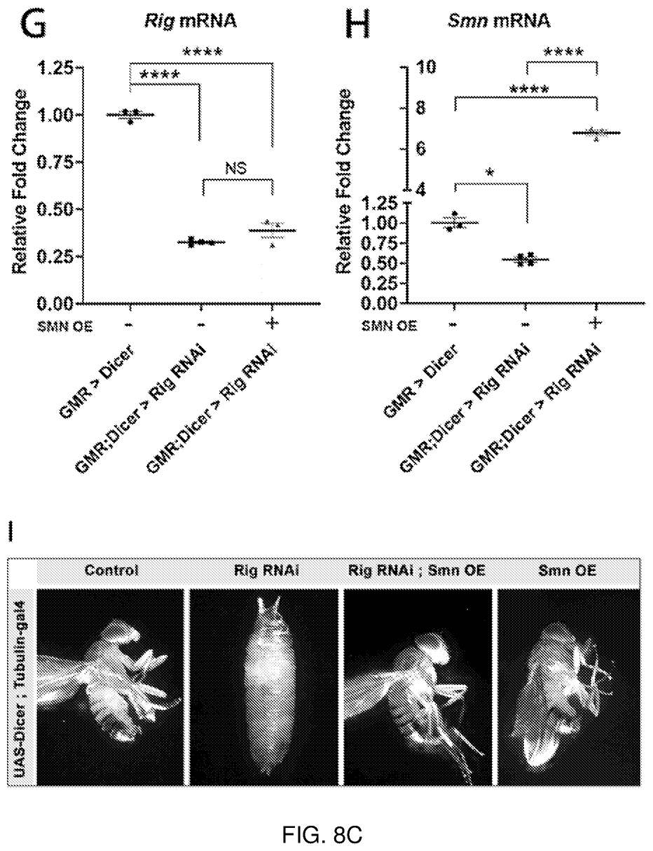

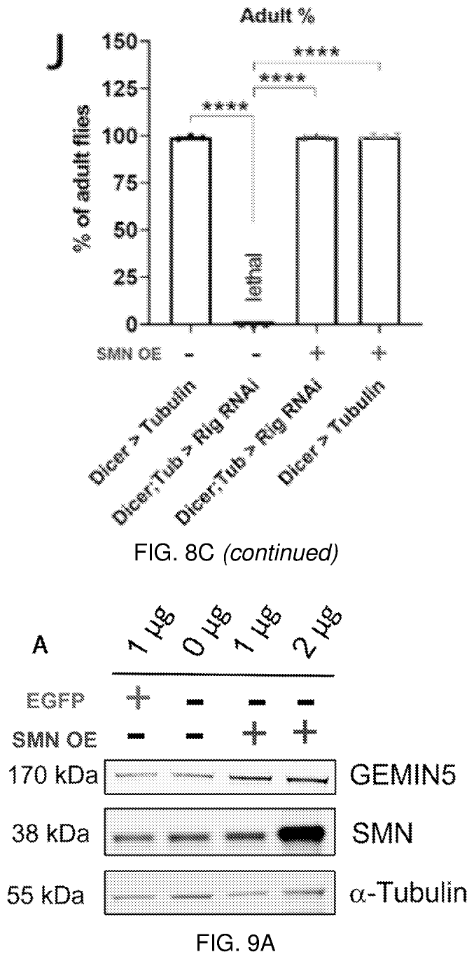

To investigate if interacting partners of GEMIN5 could compensate for the neurodegeneration associated with loss of GEMIN5 (Rigor mortis, the Drosophila homologue of human GEMIN5) in vivo, a screen of Drosophila SMN complex members was completed and their impact on the effects of Rigor mortis (Rig) knock down (KD) was assessed (FIG. 8A (A)). Utilizing the glass multiple reporter (GMR), Rig RNAi-expressing animals were crossed with control, Luciferase (LUC) over expression (OE) (positive control to rule out gal4 dilution), Smn RNAi, Smn OE, and Gemin2 OE-mediated fly lines to drive targeted expression of the transgenes to Drosophila eyes. UAS-dicer was used to enhance the KD of Rig and resulted in external eye degeneration and ommatidial disorganization phenotypes as compared to control animals (FIG. 8A (B)). Overexpression of Luciferase and KD of Smn had no effect on the external eye degeneration of Rig KD animals (FIG. 8B (C)-(D)). Ectopic expression of Drosophila Smn suppressed the external eye degeneration phenotypes in Rig RNAi expressing animals (FIG. 8B (E)). Contrary to Smn over expression, it was found that ectopic expression of Drosophila Gemin2 significantly exacerbated eye toxicity in Rig RNAi expressing animals as compared to controls. In addition, ectopic expression of Smn significantly restored the decreased eye size of Rig RNAi expressing animals (FIG. 8B (F)). Further, it was found that Smn over expression continued to suppress Rig RNAi induced external eye degeneration over time. A similar suppression in Rig phenotype among both males and females was observed.

The expression levels of Rig and Smn in the Rig RNAi-expressing animals via qPCR and WB were examined. It was found that endogenous levels of both Rig mRNA (decreased by approximately 65-70%) and protein (FIG. 8C (G)) were significantly reduced upon KD of Rig alone, as compared to control. In addition, Smn mRNA (decreased by approximately 50%) and protein were also significantly reduced in Rig KD animals (FIG. 21(H), FIG. 21(F)). WB and qPCR confirmed ectopic expression of Smn in Rig KD animals and had no effect on the efficacy of silencing expression of Rig via RNA interference (FIGS. 21(G)-(H), FIGS. 22(D)-(F)). Thus, GEMIN5 appeared to be needed for motor function, as patients with homozygous mutations manifest neurological symptoms at very early stages (Kour et al., 2021; Saida et al., Clin Genet, 2021, 100 (6): 722-730; Rajan et al., Front Cell Dev Biol, 2022, 10:783762). Tubulin-GAL4 was used to drive ubiquitous expression of Rig RNAi in flies ectopically expressing Smn (FIG. 8C (I)). A complete pupal lethality of Rig KD animals compared to controls was observed, were severe developmental defects were associated with loss of Rig. Upregulation of Smn significantly ameliorated the developmental defects observed in Rig KD animals, with up to almost 100% of animals eclosing to the adult stage (FIG. 8C (J)). Together, these results indicated that upregulation of Smn in Rig KD animals rescued morphological defects and motor dysfunction associated with the loss of Gemin5 in vivo.

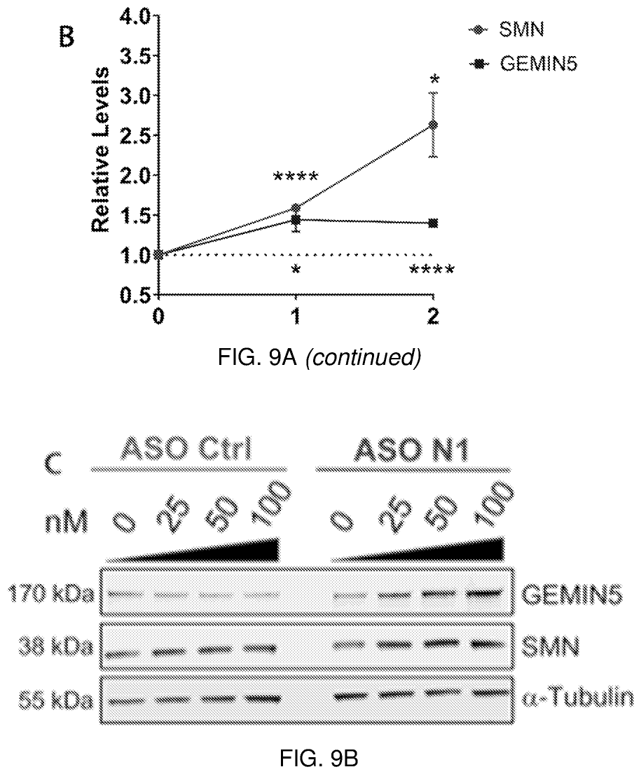

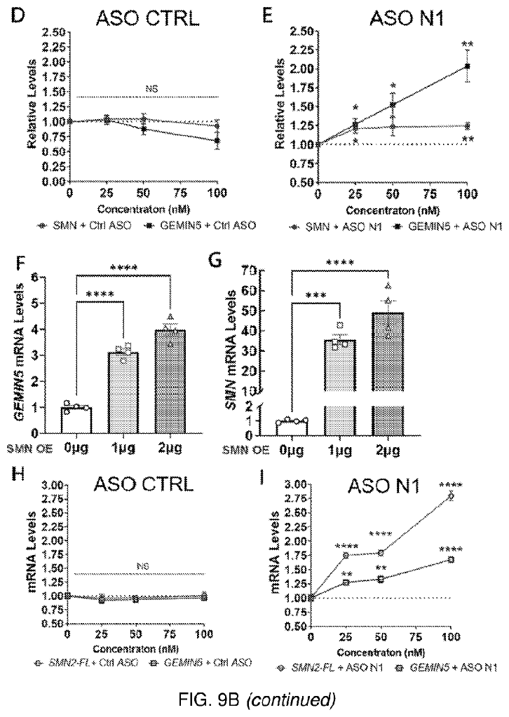

HEK293T cells were transfected with EGFP control and EGFP-SMN (dual promoter lentiviral construct resulting in coexpression of SMN and EGFP separately) in a dose-dependent manner and the protein expression of GEMIN5 and SMN via WB was measured (FIG. 9A (A)). It was found that the protein levels of endogenous GEMIN5 (FIG. 9A (B)) were significantly increased upon ectopic expression of SMN. In addition, it was found that EGFP-SMN also increased the fluorescent intensity of both SMN and GEMIN5 in HEK293T cells. HA-GEMIN2 and HA-GEMIN4 in HEK293T cells was ectopically expressed and no significant changes in GEMIN5 expression compared to controls was observed.

HEK293T cells were treated with a scrambled control antisense oligonucleotide ASO (ASO Ctrl) and with Nusinersen (ASO N1) at 3 different concentrations (25 nanomolar (nM), 50 nM, 100 nM) and assessed the protein levels of GEMIN5 and SMN via WB (FIG. 9B (C)). Treatment with ASO N1 resulted in a significant increase in GEMIN5 and SMN (FIG. 9B (E)), compared to controls (FIG. 9B (D)). In addition, 25 nm of Cy3 ASO N1 increased the fluorescent intensity of both SMN and GEMIN5 in HEK293T cells. To determine the effectiveness of the N1-targeting ASO, PCR analysis was performed to assess the percentage of SMN2 exon 7 inclusion in HEK293T cells after being treated with 0, 25, 50, and 100 nM of the control and ASO N1 ASOs. Treatment with ASO N1 produced a dose-dependent increase in the inclusion of SMN2 exon 7 with the highest inclusion percentage being 80% at 100 nM for ASO N1 compared to 40% for the control.

To assess if upregulation of GEMIN5 was restricted to the protein level or also had an effect at the mRNA level in response to SMN expression, the mRNA levels of GEMIN5 and SMN via qPCR were measured. It was found that the mRNA levels of GEMIN5 (FIG. 9B (F)) were also increased in response to EGFP-SMN (FIG. 9B (G)). The mRNA expression of GEMIN5 and SMN2 after control ASO (FIG. 9B (H)) and ASO N1 treatment in HEK293T cells was also assessed. A dose dependent increase in both GEMIN5 and SMN2 mRNAs in response to ASO N1 treatment was observed, where upregulation of both SMN transcripts (SMN1 & SMN2) in HEK293T cells influences the overall expression of GEMIN5.

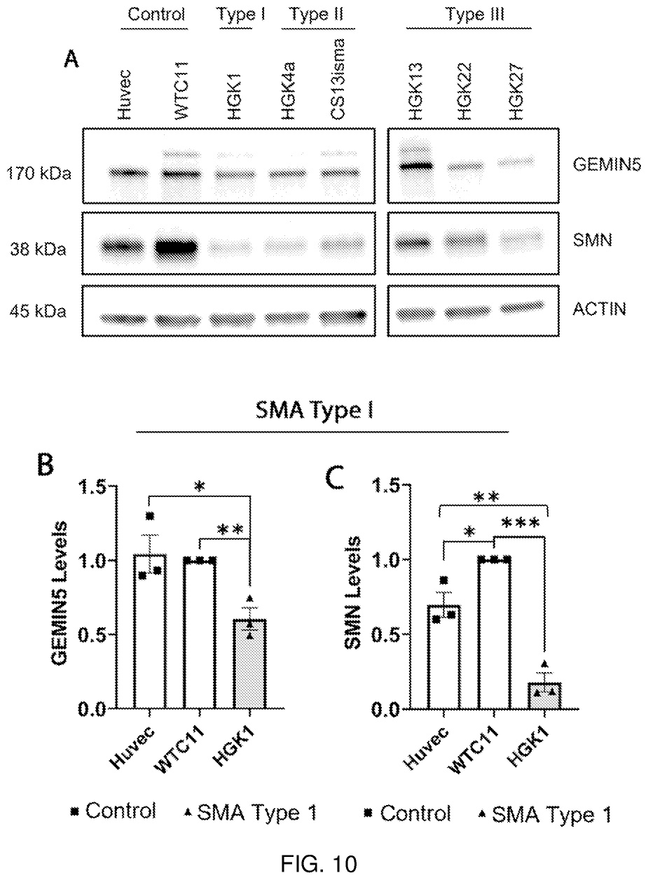

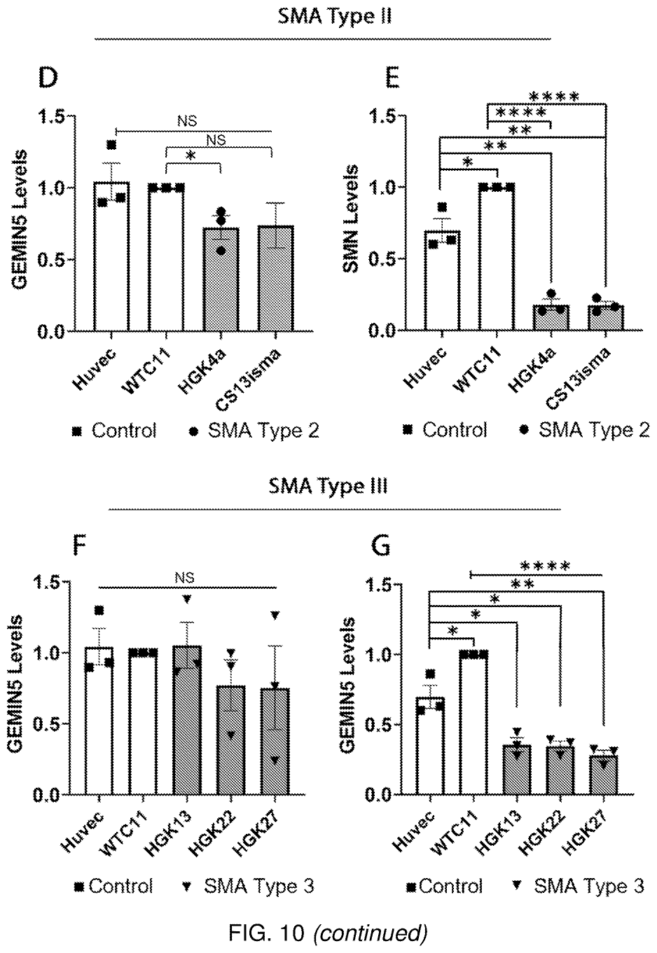

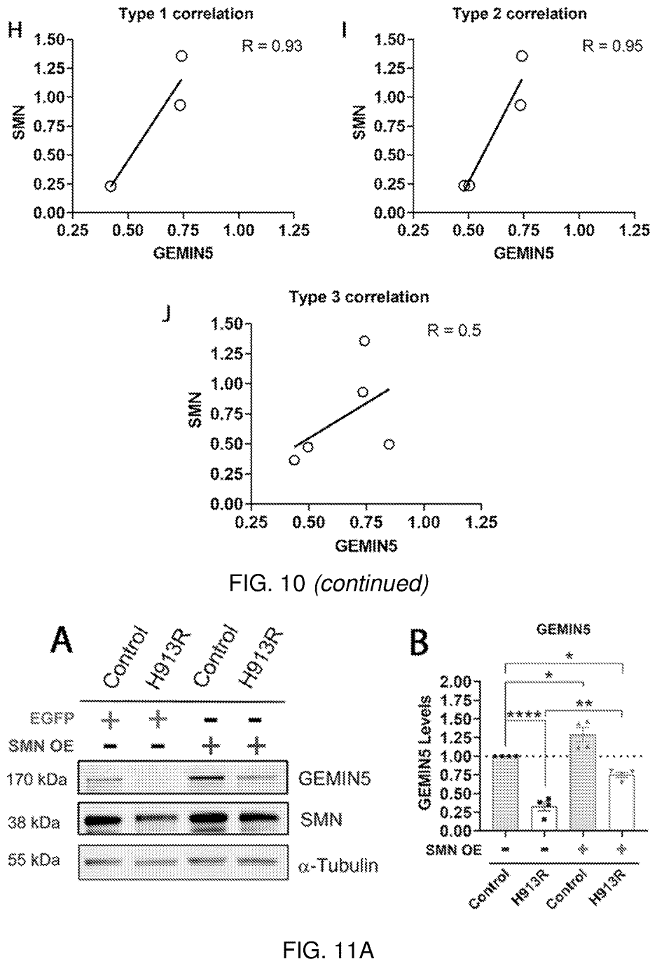

The levels of GEMIN5 and SMN in 6 SMA patient iPSC samples harboring LOF mutations in the SMN gene measured. Two healthy control iPSC lines and 6 SMA patient iPSC lines (across 3 types of SMA) were differentiated into motor neurons and WB analysis was conducted (FIG. 10(A)). The SMA type 1 neurons displayed a significant decrease in the levels of both GEMIN5 (approximately a 50% reduction) (FIG. 10(B)) and SMN (approximately a 85% reduction), as compared to the 2 control motor neuron samples. In addition, GEMIN5 (approximately a 30% reduction) and SMN (approximately 80% reduction) (FIG. 10(E)) were also reduced in both SMA type 2 motor neurons, as compared to the controls. Lastly, all SMA type 3 motor neuron samples did not result in a significant decrease in GEMIN5 expression (FIG. 10(F)), while displaying a drastic reduction in SMN (approximately a 60% reduction) (FIG. 10(G)) as compared to the healthy controls. Thus, the effect of reduced GEMIN5 expression in SMA motor neurons is dosage-dependent, as over a 75% decrease in SMN expression was needed to significantly influence GEMIN5 expression. A strong positive correlation was observed between the levels of SMN and GEMIN5 in the SMA type 1 and type 2 motor neurons (FIG. 10 (H, I), while only exhibiting a moderate positive correlation in the SMA type 3 motor neurons, indicating an association between SMN and GEMIN5 expression.

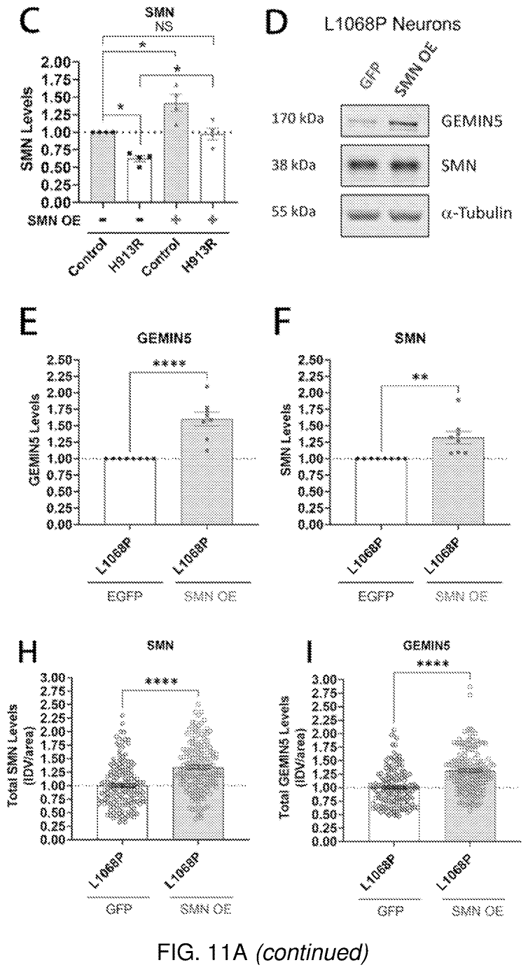

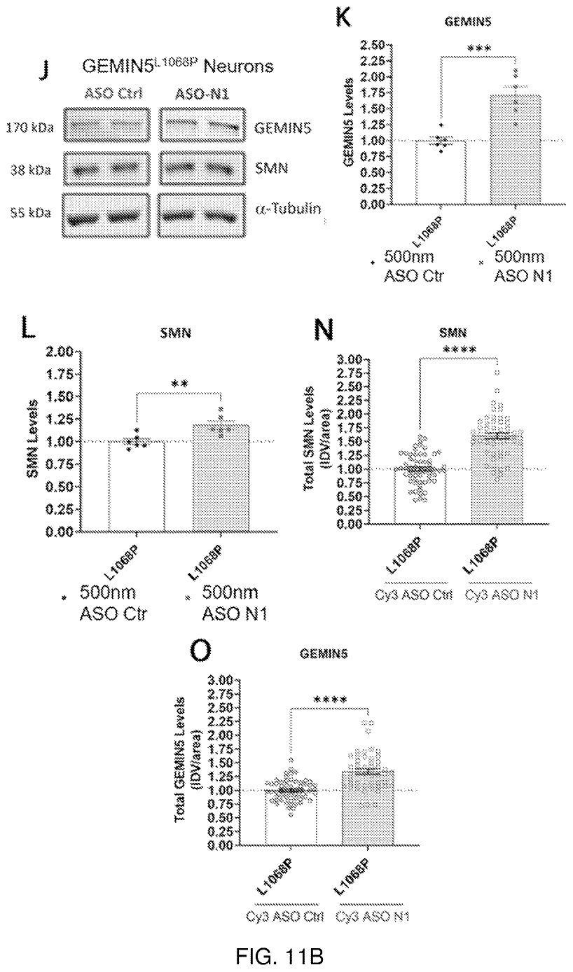

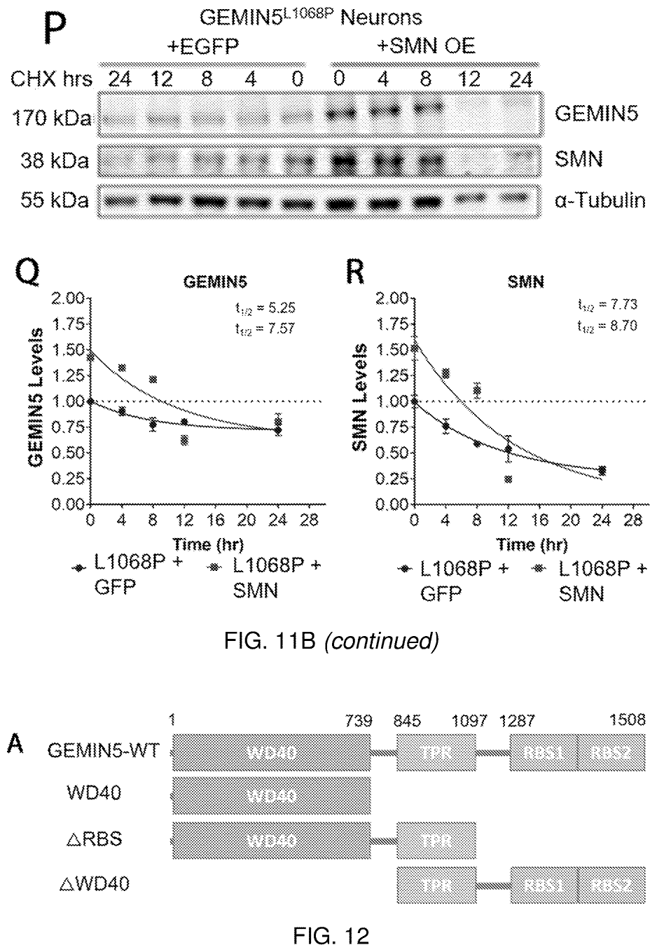

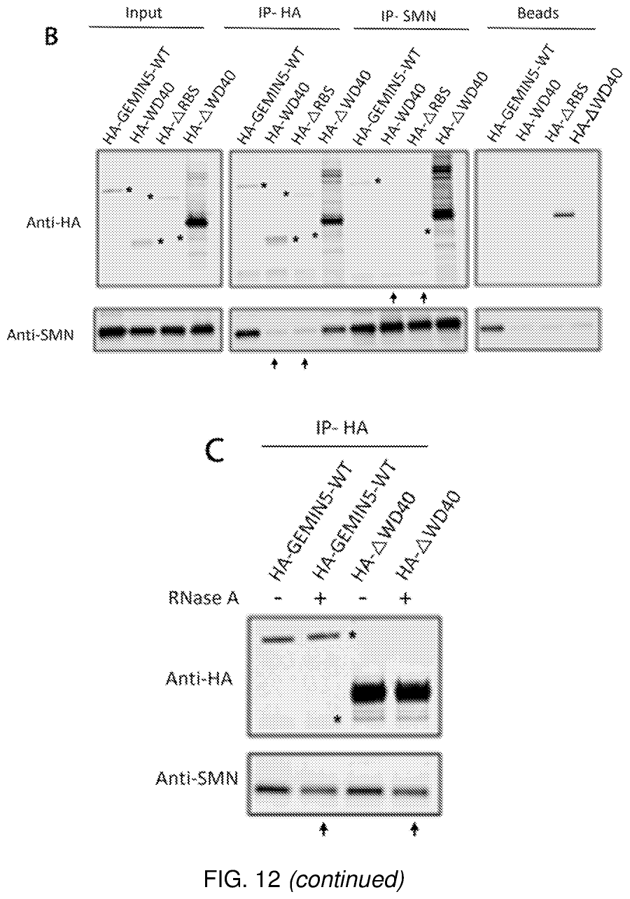

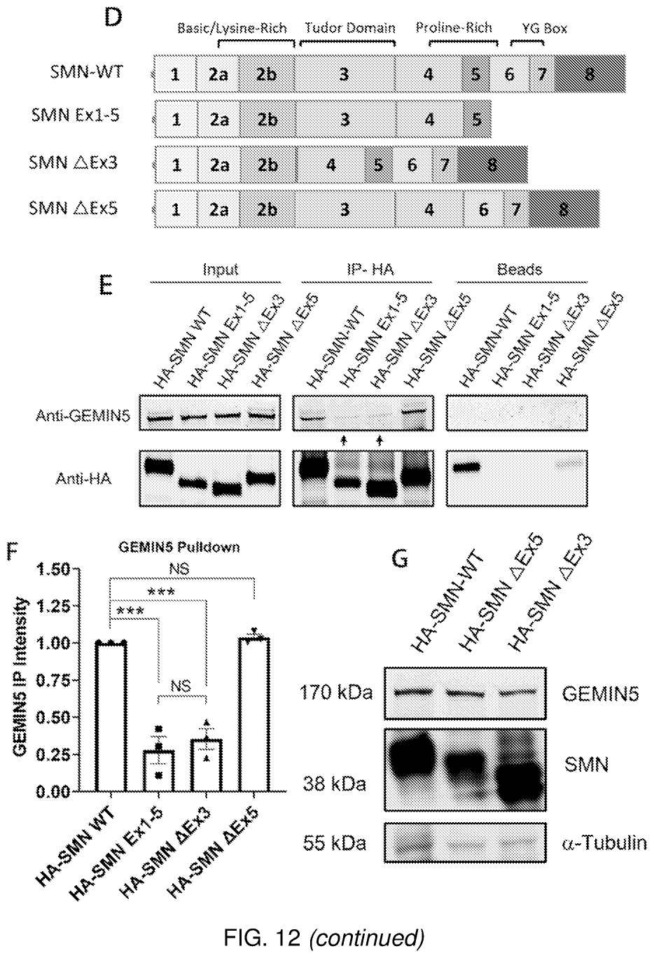

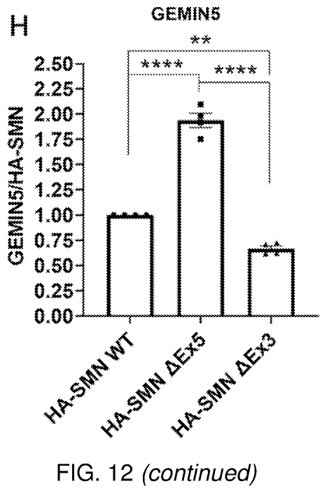

Utilizing a lentiviral expression system, EGFP and EGFP-SMN was expressed in mutant GEMIN5^{H913R} iPSC derived neuronal cells and the expression levels of GEMIN5 and SMN via WB were assessed. In GEMIN5^{190 913R} derived neurons expressing EGFP control, it found the expression levels of GEMIN5 was reduced up to approximately 70-80% compared to the unaffected controls (FIG. 11(A)). A reduction in the protein levels of SMN in mutant GEMINSH913R neurons expressing EGFP as compared to controls was also observed. Lentiviral expression of SMN (increased by approximately 40%) (FIG. 11A (C)) in mutant GEMIN51913R neurons significantly increased the protein levels of GEMINS (FIG. 11A (B)), as compared to GEMIN^{51913R} neurons expressing EGFP control. The effect of lentiviral SMN on GEMIN5 in GEMIN5^{L1068P} iPSC derived neurons was evaluated (FIG. 11A (D)). Lentiviral expression of SMN (increased by approximately 30%) (FIG. 11A (F)) in mutant GEMIN5^{L1068P} neurons significantly increased the protein levels of mutant GEMIN5 (FIG. 11A (E)), as compared to GEMIN5^{L1068P} neurons expressing EGFP control. To gain further insights into mechanisms behind SMN's influence on mutant GEMIN5, the effect of SMN expression on the localization pattern of GEMIN5 in mutant GEMIN5^{L1068P} iPSC derived neuronal cells was assessed (FIG. 11A (G, tentionally omitted)). A significant increase in total SMN (FIG. 11A (H)) and GEMIN5 (FIG. 11A (I)) fluorescent intensity in mutant GEMIN5^{L1068P} neuronal cells expressing lentiviral EGFP-SMN as compared to mutant GEMIN5^{L1068P} neuronal cells expressing EGFP control was found. In neurons harboring GEMIN5^{190 913R}, it was found that the levels of GEMIN5 mRNA were increased in response to lentiviral EGFP-SMN in both control and mutant GEMIN5 neuronal cells. Thus, the effect of SMN upregulation on GEMIN5 was determined to be at the transcriptional and translational level.

GEMIN5^{L1068P} neurons were treated with 500 nM of ASO N1 and scrambled control ASO and a significant increase in the expression levels of both GEMIN5 (FIG. 11B (K)) and SMN (FIG. 11B (L)) was observed. To assess the efficacy of ASO NI administration, PCR analysis was performed on the GEMIN5L106SP neurons and it was found that ASO N1 administration at 500 nM reduced exon 7 SMN2 skipping from 50% to 25%. This resulted in about a 25% increase in SMN2 mRNA, which also resulted in a 25% increase in GEMIN5 mRNA (FIG. 29(F)). The impact of ASO NI administration on the localization patterns of endogenous SMN and GEMIN5 in mutant GEMIN511068P iPSC derived neuronal cells was investigated (FIG. 11B (M, intentionally omitted)). Administration of Cy3 ASO N1 increased the fluorescent intensity of both SMN (FIG. 11B (N)) and GEMIN5 (FIG. 11B (O)) in mutant GEMIN5^{L1068P} jPSC derived neuronal cells compared to the scrambled Cy3 ASO control. Thus, ASO NI exerted similar effects as lentiviral SMN in mutant GEMIN5 iPSC derived neurons and increased the expression of both SMN and GEMIN5 at the mRNA and protein level.

To examine other possible underlying mechanisms responsible for the increased protein levels of GEMIN5 in response to SMN expression, GEMIN5's protein stability between mutant GEMIN5^{L1068}P neurons expressing EGFP and EGFP-SMN were compared (FIG. 11B (P)). The protein expression of GEMIN5 and SMN after 0, 4, 8, 12, and 24 hours of cycloheximide (CHX) treatment was assessed. A steady decrease in GEMIN5 expression was observed in neurons expressing EGFP control after 2 hours of CHX treatment, whereas a higher expression of GEMIN5 was observed up to 8 hours after CHX treatment in response to lentiviral SMN compared to controls (FIG. 11B (Q)). SMN protein levels showed a steady reduction after two hours of CHX treatment in EGFP expressing controls, whereas a higher expression of SMN was observed up to 8 hours after CHX treatment in neurons expressing lentiviral SMN (FIG. 11B (R)).

To assess the copy numbers of both SMN1 and SMN2 in the unaffected parent and the GEMIN5^{L1068P} patient, DNA was extracted from the patient iPSCs and conducted simplex Real-Time PCR SMN copy number quantification (Passon et al., Genet Test Mol Biomarkers, 2009, 13 (1): 37-42; Stabley et al., Mol Genet Genomic Med, 2015, 3 (4), 248-257). It was found that both the unaffected parent and mutant GEMIN⁵¹¹⁰⁶⁸P individual harbor 2 copies of both SMN1 and SMN2. Thus, the reduction in SMN protein levels in the GEMIN5L1068P individual was determined to be caused by the loss-of-function biallelic mutations in GEMIN5. The upregulation of SMN influenced the levels of GEMIN5 in mutant GEMIN5 iPSCs.

To identify the domains responsible for GEMIN5 and SMN interaction, HEK293T cells were co-transfected with full length HA-GEMIN5-WT, HA-WD40 (n-terminus only), HA-ΔRBS (deleted c-terminus), HA-ΔWD40 (deleted n-terminus), and EGFP-SMN and tested via coimmunoprecipitation (FIG. 12(A)). The disruption of the c-terminus of GEMIN5, which harbors two RNA-binding sites, abolished GEMINS's interaction with SMN, indicating that functional RNA-binding sites of GEMIN5 are required for efficient interaction with SMN protein (FIG. 12(B)). To assess if this interaction was RNA dependent, HA-GEMIN5-WT and HA-ΔWD40 immunoprecipitated lysates were treated with RNase A. It was found that RNase A slightly decreased the interaction between GEMIN5 and SMN. Thus, their interaction was partially regulated through RNA (FIG. 12(C)). To identify the SMN protein domain responsible for the interaction with GEMIN5, HA-SMN-WT (full length), HA-SMN-Ex1-5 (lacking exon 7-main product of SMN2 gene), HA-SMN-AEx3 (deletion of the SMN Tudor domain), and HA-SMN-ΔEx5 (deletion of the Proline-Rich region of SMN) were transfected in HEK293T cells and tested by immunoprecipitation (FIG. 12(D)). Immunoprecipitation with the SMN2 product (HA-SMN-Ex1-5 which lacks E7 of SMN) and deletion of the SMN Tudor domain (HA-SMN-ΔEx3) of SMN completely abolished the interaction with GEMIN5 (FIG. 12(E)-(F)). To assess if the Tudor domain was also required for SMN regulating GEMIN5 expression, HEK cells were transfected with HA-SMN-WT, HA-SMN-ΔEx5, and HA-SMN-AEx3 and analyzed the protein expression of both GEMIN5 and SMN (FIG. 12(G)). HA-SMN-WT and HA-SMN-ΔEx5 both increased the levels of endogenous SMN, which resulted in an increase in GEMIN5 (FIG. 12(H)). HA-SMN-ΔEx3 failed to increase the levels of endogenous GEMIN5. Therefore, the Tudor domain of SMN was hypothesized as being present for GEMIN5 binding and expression.

The presence of nuclear U snRNPs in GEMIN⁵¹¹⁰⁶⁸P neuronal cells were examined by immunostaining for SmB (Y12). A decrease in nuclear U snRNP expression in GEMIN5^{L1068P} neurons as compared to control was observed. A significant increase in nuclear U snRNP in both control and mutant GEMIN5^{L1068P} neurons expressing SMN was observed. Since a decrease in imported U snRNPs in mutant GEMIN5 neurons was observed, the presence of CBs in mutant GEMIN5^{H913R} neuronal cells was also examined. GEMIN5H913R neuronal cells expressing EGFP displayed a significant reduction in the average number of CBs per cell and the percentage of neuronal cells that harbored CBs, compared to control. Lentiviral expression of SMN restored the loss of CB formation and increased the percentage of neuronal cells harboring CBs in mutant GEMIN59913R neuronal cells. There appeared to be no obvious localization of GEMIN5 into the CB structures. The snRNP core was reconstituted in vitro by using transcribed 3′Cy3-biotinylated-U1snRNA and cytoplasmic extracts from control and mutant GEMIN5^{L1068P} neurons transduced with EGFP-SMN and treated with 500 nM of scramble control and ASO N1 ASO. Cytoplasmic extract was used from HEK293T cells transfected with scramble shRNA and GEMIN5 shRNA as a positive control (Kour et al., 2021). Assembly formation was drastically reduced in GEMIN5^{L1068P} neurons with the scrambled control ASO compared to control neurons. A partial rescue in assembly formation in GEMIN5^{L1068P} neurons transduced with lentiviral SMN and treated with ASO N1 ASO was also observed.

The UAS/gal4 system and used Tubulin-GS-gal4 (GeneSwitch) induced by a mild dose of RU486 to induce ubiquitous expression of Rig KD throughout larval development was used to determine if there were also snRNP core assembly phenotypes associated with loss of Gemin5 in vivo. Drosophila larval brains of control, Rig KD, and Rig KD Smn over expressing animals were dissected in the ventral nerve cord (VNC) and immunostained for Drosophila U snRNP (dSmb) and cell nuclei (DAPI. Similarly to the mutant GEMIN5 iPSC derived neurons, it was found that dSmB was drastically reduced in Rig KD animals compared to control. It was investigated if ectopic expression of Smn rescued dSmB expression in Drosophila brains of Rig KD animals and a noticeable rescue in dSmB expression was observed. The expression of Drosophila Coilin in the brains of control, Rig KD, and Rig KD Smn over expressing larvae was analyzed. A drastic reduction in dCoilin expression and CB formation in Rig KD animals compared to control was observed. Upregulation of Smn restored dCoilin expression in Drosophila brains of Rig KD animals.

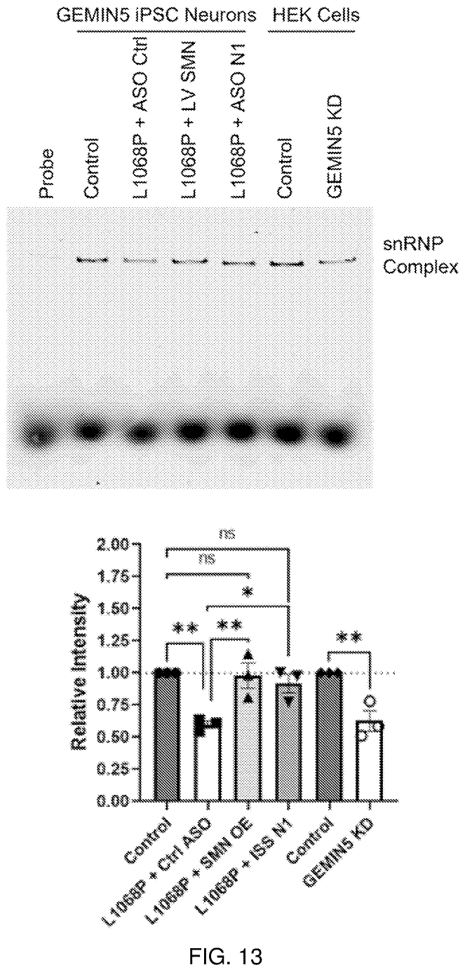

GEMIN5 is an essential component of the SMN complex that is required for biogenesis of the spliceosome snRNP core. Upon final completion of snRNP core assembly in the cytoplasm, the snRNPs are imported into the nucleus and transiently localize in nuclear body structures termed Cajal Bodies (CBs). More importantly, disruption of snRNP core assembly resulting from the loss of SMN complex proteins leads to a destabilized SMN complex and significant disruptions in CB formation. These findings prompted us to ask if mutations in GEMIN5 disrupt snRNP biogenesis and import into CBs. First, we examined the presence of nuclear U snRNPs in GEMIN5^{L1068P} neuronal cells by immunostaining for SmB (Y12). We observed a drastic decrease in nuclear U snRNP expression in GEMIN5L1068P neurons as compared to control. These findings prompted us to ask if lentiviral SMN had any effect on U snRNP expression in the GEMIN5^{L1068P} neurons. Interestingly, we observed a significant increase in nuclear U snRNP in both control and mutant GEMIN5^{L1068P} neurons expressing SMN. Since we saw a decrease in imported U snRNPs in mutant GEMIN5 neurons, we also observed for the presence of CBs in mutant GEMIN5^{H913R} neuronal cells. GEMIN5^{H913R} neuronal cells expressing EGFP displayed a significant reduction in the average number of CBs per cell and the percentage of neuronal cells that harbored CBs compared to control. Surprisingly, lentiviral expression of SMN restored the loss of CB formation and increased the percentage of neuronal cells harboring CBs in mutant GEMIN5^{H913R} neuronal cells. There appeared to be no obvious localization of GEMIN5 into the CB structures. Next, we wanted to assess if SMN upregulation was sufficient to rescue mature snRNP assembly in GEMIN5^{L1068P} neurons. To investigate this, we reconstituted the snRNP core in vitro by using transcribed 3′Cy3-biotinylated-UlsnRNA and cytoplasmic extracts from control and mutant GEMIN5^{L1068P} neurons transduced with EGFP-SMN and treated with 500 nM of scramble control and ASO N1 ASO (FIG. 13 (top). We also used cytoplasmic extract from HEK293T cells transfected with scramble shRNA and GEMIN5 shRNA as a positive control. Assembly formation was drastically reduced in GEMIN5L1068P neurons with the scrambled control ASO compared to control neurons. We observed a significant rescue in assembly formation in GEMIN5^{L1068P} neurons transduced with lentiviral SMN and treated with the ASO N1 (FIG. 13 (bottom).

To determine if there are also snRNP core assembly phenotypes associated with loss of Gemin5 in vivo, we utilized the UAS/gal4 system and used Tubulin-GS-gal4 (GeneSwitch) induced by a mild dose of RU486 to induce ubiquitous expression of Rig KD throughout larval development. First, we dissected Drosophila larval brains of control, Rig KD, and Rig KD Smn over expressing animals in the ventral nerve cord (VNC) and immunostained for Drosophila U snRNP (dSmb) and cell nuclei (DAPI). Similarly to the mutant GEMIN5 iPSC derived neurons, we found that dSmB was drastically reduced in Rig KD animals compared to control. We therefore investigated if ectopic expression of Smn rescued dSmB expression in Drosophila brains of Rig KD animals and observed a noticeable rescue in dSmB expression. Next, we investigated the expression of Drosophila Coilin in the brains of control, Rig KD, and Rig KD Smn over expressing larvae. We observed a drastic reduction in dCoilin expression and CB formation in Rig KD animals compared to control. Surprisingly, upregulation of Smn restored dCoilin expression in Drosophila brains of Rig KD animals. . . . These results suggest that mutations and loss of Gemin5 in vivo disrupt snRNP assembly and upregulation of SMN partially rescues snRNP phenotypes associated with GEMIN5-mediated disease.

Thus, mutations and loss of Gemin5 in vivo disrupted snRNP assembly and the upregulation of SMN partially rescued snRNP phenotypes associated with GEMIN5-mediated disease.

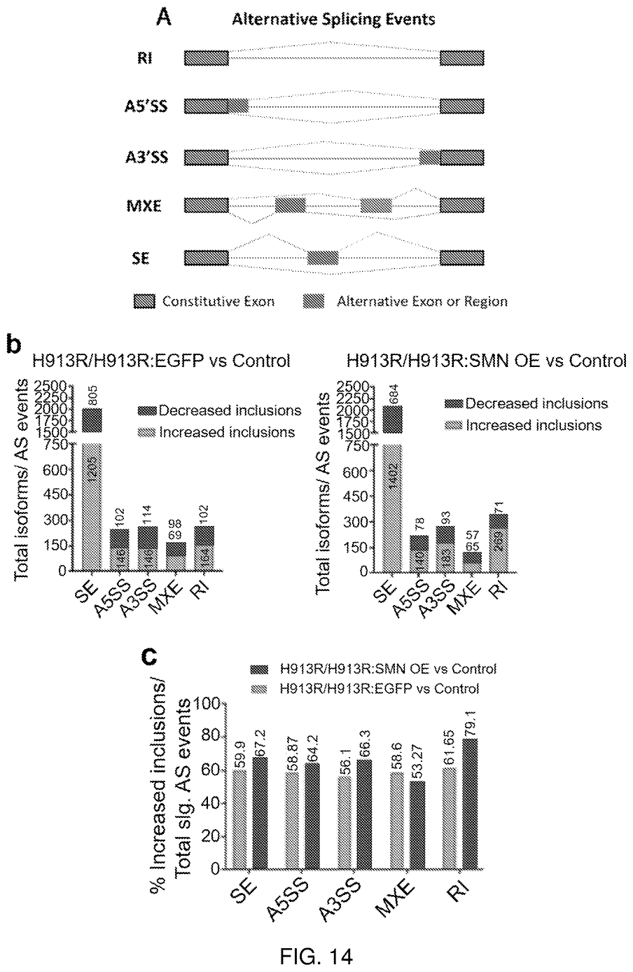

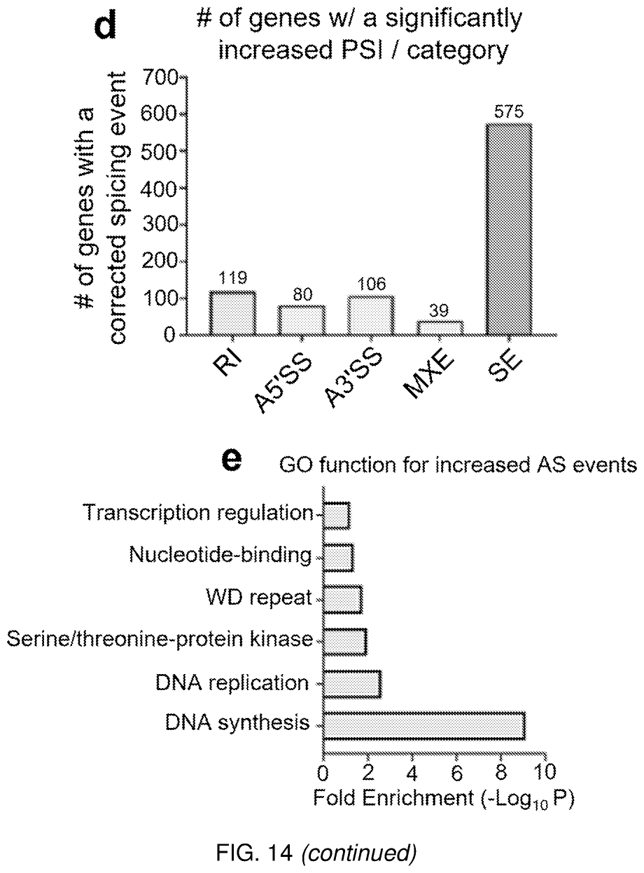

The results above suggest SMN upregulation rescues snRNP defects exhibited in GEMIN5-mediated disease. Since snRNP biogenesis is a key driver of RNA splicing events, we investigated the alternative splicing (AS) profile of mutant GEMIN5^{H913R} neurons with lentiviral expression of EGFP and EGFP-SMN compared to control neurons using rMATS. rMATS calculates the inclusion of a given differentially expressed exon as percent spliced-in (PSI or Y) and assesses the fraction of a gene's mRNA across five main alternative splicing patterns, retained intron (RI), alternative 5′ splice site (A5SS), 3′ alternative splice site (A3SS), mutually exclusive exon (MXE), and exon skipping (SE) (FIG. 14(A)). Using an FDR of <0.05 and a ΔY>5% we identified a total of 2,950 significant splicing events (1,758 with a significantly increased Y and 1,192 with a significantly decreased P in mutant GEMIN5H913R neurons. Exon skipping represented the largest number of AS events between the two groups with a total of 2,009/2,950 (68.1%) (FIG. 14(B)). Importantly, after lentiviral expression of SMN in the mutant GEMIN5^{H913R} neurons, we found a higher number of significant AS events with an increased inclusion event per each splicing category compared to controls (FIG. 7b). Additionally, we found a lower number of significant AS events exhibiting a decreased inclusion per splicing category after lentiviral SMN expression in mutant GEMIN5H913R neurons compared to controls (FIG. 14(B)). Exon skipping resulted in a 7.3% increase in inclusions followed by an increase in A5′SS (5.3%), A3′SS (10.2%), and RI (17.45%) AS events in GEMIN5^{H913R} neurons with SMN vs. control compared to GEMIN5H913R neurons with EGFP vs. control (FIG. 14(C)). Additionally, Exon skipping resulted in a lower percentage of decreased inclusions (7.4%) followed by a lower percentage of significant A5′SS (5.4%), A3′SS (7.1%), and RI (17.42%) events in GEMIN5^{H913R} neurons with SMN vs. control compared to GEMIN5^{H913R} neurons with BGFP vs. control. Out of the 5 AS categories, we identified 919 total genes that exhibited a significant increase in inclusion (or rescued splicing event) with 575 genes of those genes exhibiting rescued exon skipping events after lentiviral SMN (FIG. 14(D)). Gene ontology analysis was applied to assess the biologically relevant pathways impacted by the genes that exhibited an increase in inclusion after SMN expression. The most affected biological pathways involved DNA synthesis, DNA replication, serine/threonine-protein kinases, WD-repeat domain, nucleotide binding, and transcriptional regulation (FIG. 14(E)). Collectively, since SMN upregulation exhibited the largest effect on exon skipping, we further analyzed increased inclusion events in this category. We identified 3 transcripts in this data set that were also differentially spliced in response to biallelic mutations in GEMIN510. Using rmatssashimplot software, we visualized the exon skipping events of these 3 genes, FNBP1, EHBP1, and SPTSSB and found them all to result in increased exon inclusion after SMN upregulation. Of note, FNBP1 and SPTSSB have recently been implicated in the motor neuron disease amyotrophic lateral sclerosis (ALS) and have not been linked with SMA or other neurodevelopment diseases. Overall, SMN expression significantly restored the inclusion of alternatively spliced isoforms in mutant GEMIN5 derived iPSC neurons.

Therefore, it was demonstrated that SMN is a genetic regulator of GEMIN5-mediated disease in mammalian cells, mutant GEMIN5 iPSC neurons, and in vivo. Upregulation of SMN increased the expression level of GEMIN5 in mammalian cells and mutant GEMIN5 iPSC neurons. SMN modified GEMINS-mediated neuropathologies and it was demonstrated that SMN-upregulating therapies in GEMINS-mediated disease was therapeutic for restoring the snRNP complex defects exhibited by mutant GEMIN5.

Having described this invention, it will be understood to those of ordinary skill in the art that the same can be performed within a wide and equivalent range of conditions, formulations and other parameters without affecting the scope of the invention or any embodiment thereof. References incorporated herein by reference are incorporated for their technical disclosure and only to the extent that they are consistent with the present disclosure.

1. A method for treating a neurodevelopmental disorder, amyotrophic lateral sclerosis, or spinal muscular atrophy in a patient, comprising administering to the patient a therapeutic agent in an amount effective to treat the neurodevelopmental disorder,
wherein the therapeutic agent is chosen from one or more of the following:
an antisense oligonucleotide (ASO) targeting SMN1 or SMN2;
a mRNA splicing modifier, a pharmaceutically acceptable salt thereof, or a solvate thereof; or
a gene therapy for expressing an SMN1 or SMN2 transcript;
wherein when the patient has a neurodevelopmental disorder, the patient having the neurodevelopmental disorder comprises one or more biallelic variants in GEMIN5 and one or more of a developmental delay, motor dysfunction, cerebellar atrophy, ataxia, and/or hypotonia.

2. The method of claim 1, wherein the patient comprises one or more biallelic variants in GEMIN5 and wherein the biallelic variant is, relative to SEQ ID NO: 2: p. (Leu1068Pro), p. (Ala1007Thr), p. (His923Pro), p. (His913Arg), p. (Asp704Glu), p. (Gly683Asp), p. (Ser1000Pro) p. (Tyr1282His), p. (Cys1205Trp), p. (Arg1014Gln), p. (Arg1016Cys), p. (Met485Hfs*27), p. (Arg1016Cys), p. (Arg899Pfs*3), p. (Trp373*), p. (Ala994Val), p. (Pro594Arg), p. (Ser543Gly), or combinations thereof.

3. The method of claim 1, wherein the patient has a developmental delay, motor dysfunction, cerebellar atrophy, and ataxia.

4. The method of claim 1, wherein the administration of the ASO, the mRNA splicing modifier, the gene therapy, or combinations thereof upregulates survival motor neuron (SMN) protein.

5. The method of claim 5, wherein the upregulation of the SMN protein increases the expression of GEMIN5 protein.

6. The method of claim 1, wherein the therapeutic agent is an ASO an antisense oligonucleotide (ASO) targeting SMN1 or SMN2.

7. The method of claim 6, wherein the ASO is administered parenterally.

8. The method of claim 6, wherein the ASO is nusinersen.

9. The method of claim 8, wherein from at least 0.5 mg/mL to at least 3 mg/mL of nusinersen, or a pharmaceutically-acceptable salt or solvate thereof, is administered to the patient in multiples doses.

10. The method of claim 1, wherein the therapeutic agent is an mRNA splicing modifier, a pharmaceutically acceptable salt thereof, or a solvate thereof.

11. The method of claim 10, wherein the mRNA splicing modifier is an SMN2 mRNA splicing modifier.

12. The method of claim 11, wherein the SMN2 mRNA splicing modifier comprises a compound having the formula (I): 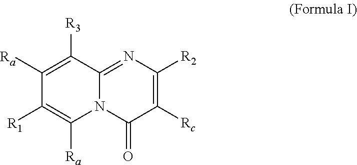
or a free acid, free base, a chloride salt, hydrobromide salt, hydrochloride salt, dihydrochloride salt, acetate salt, trifluoroacetate salt, trifluoroacetic acid salt, isotopologue, stereoisomer, racemate, enantiomer, diastereomer or tautomer thereof, wherein:
R₁ is a heterocyclyl, wherein, the heterocyclyl is optionally substituted with one, two or three R₃ substituents and optionally, with one additional R₄ substituent; or, wherein, heterocyclyl is optionally substituted with one, two, three or four R₃ substituents, where R₃ is, in each instance, independently selected from cyano, halogen, hydroxy, oxo, C₁₋₈alkyl, halo-C₁₋₈alkyl, C₁₋₈alkyl-carbonyl, C₁₋₈alkoxy, halo-C₁₋₈alkoxy, C₁₋₈alkoxy-C₁₋₈alkyl, C₁₋₈alkoxy-carbonyl, amino, C₁₋₈alkyl-amino, (C₁₋₈alkyl)₂-amino, amino-C₁₋₈alkyl, C₁₋₈ alkyl-amino-C₁₋₈alkyl, (C₁₋₈alkyl)₂-amino-C₁₋₈alkyl, amino-C₁₋₈alkyl-amino, C₁₋₈alkyl-amino-C₁₋₈ alkyl-amino, (C₁₋₈alkyl-amino-C₁₋₈alkyl)₂-amino, (C₁₋₈alkyl)₂-amino-C₁₋₈alkyl-amino, [(C₁₋₈alkyl)₂-amino-C₁₋₈alkyl]₂-amino, (C₁₋₈alkyl-amino-C₁₋₈alkyl) (C₁₋₈alkyl)amino, [(C₁₋₈alkyl)₂-amino-C₁₋₈alkyl](C₁₋₈alkyl)amino, C₁₋₈alkoxy-C₁₋₈alkyl-amino, (C₁₋₈alkoxy-C₁₋₈alkyl)₂-amino, (C₁₋₈alkoxy-C₁₋₈alkyl) (C₁₋₈alkyl)amino, C₁₋₈alkyl-carbonyl-amino, C₁₋₈alkoxy-carbonyl-amino, hydroxy-C₁₋₈alkyl, hydroxy-C₁₋₈alkoxy-C₁₋₈alkyl, hydroxy-C₁₋₈alkyl-amino, (hydroxy-C₁₋₈alkyl)₂-amino or (hydroxy-C₁₋₈alkyl) (C₁₋₈alkyl)amino; and R⁴ is C₃₋₁₄cycloalkyl, C₃₋₁₄cycloalkyl-C₁₋₈alkyl, C₃₋₁₄cycloalkyl-amino, aryl-C₁₋₈alkyl, aryl-C₁₋₈alkoxy-carbonyl, aryl-sulfonyloxy-C₁₋₈ alkyl, heterocyclyl or heterocyclyl-C₁₋₈alkyl; wherein, each instance of C₃₋₁₄cycloalkyl, aryl and heterocyclyl is optionally substituted with one, two or three R₅ substituents, where R₅ is, in each instance, independently selected from halogen, hydroxy, cyano, nitro, C₁₋₈alkyl, halo-C₁₋₈alkyl, C₁₋₈alkoxy, halo-C₁₋₈alkoxy, amino, C₁₋₈ alkyl-amino, (C₁₋₈alkyl)₂-amino or C₁₋₈alkyl-thio;
R₂ is heteroaryl; wherein the heteroaryl is optionally substituted with one, two or three R₆ substituents and optionally, with one additional R₇ substituent where R₆ is, in each instance, independently selected from halogen, hydroxy, cyano, nitro, C₁₋₈alkyl, C₂₋₈alkenyl, halo-C₁₋₈alkyl, hydroxy-C₁₋₈alkyl, C₁₋₈alkoxy, halo-C₁₋₈alkoxy, C₁₋₈alkoxy-C₁₋₈alkyl, amino, C₁₋₈alkyl-amino, (C₁₋₈alkyl)₂-amino or C₁₋₈alkyl-thio; and, R₇ is C₃₋₁₄cycloalkyl, C₃₋₁₄cycloalkyl-oxy, aryl, heterocyclyl or heteroaryl;
Rₐ is, in each instance, independently selected from hydrogen, halogen or C₁₋₈alkyl;
R_{b} is hydrogen, halogen, C₁₋₈alkyl or C₁₋₈alkoxy; and
R_{c} is hydrogen, halogen or C₁₋₈alkyl.

13. The method of claim 11, wherein the SMN2 mRNA splicing modifier is risdiplam, or a pharmaceutically-acceptable salt or solvate thereof.

14. The method of claim 13, wherein at least 0.15 milligrams per kilogram of body weight (mg/kg) or at least 5 milligrams of risdiplam, or a pharmaceutically-acceptable salt or solvate thereof, is administered daily to the patient.

15. The method of claim 1, wherein the therapeutic agent is the gene therapy.

16. The method of claim 15, wherein the gene therapy is administered by intravenous injection or infusion.

17. The method of claim 15, wherein the gene therapy is onasemnogene abeparvovec.

18. The method of claim 17, wherein the onasemnogene abeparvovec comprises vector genomes and at least 0.5×10¹⁴ vector genomes per kilograms of body weight is administered to the patient.

19. The method of claim 1, wherein the administration of the therapeutic agent reduces mortality or increases survival in the patient.

20. A therapeutic agent chosen from one or more of the following:
an antisense oligonucleotide (ASO) targeting SMN1 or SMN2;
a mRNA splicing modifier, a pharmaceutically acceptable salt thereof, or a solvate thereof; or
a gene therapy for expressing an SMN1 or SMN2 transcript;
for use in treating a neurodevelopmental disorder, amyotrophic lateral sclerosis, or spinal muscular atrophy in a patient, in an amount effective to treat the neurodevelopmental disorder, amyotrophic lateral sclerosis, or spinal muscular atrophy in a patient, wherein when the patient has a neurodevelopmental disorder, the patient having the neurodevelopmental disorder comprises one or more biallelic variants in GEMIN5 and one or more of a developmental delay, motor dysfunction, cerebellar atrophy, ataxia, and/or hypotonia.


SEQUENCE LISTING (33 sequences)

SEQ ID NO: 1 - RNA, 5404 nt, Homo sapiens
  source 1..5404: mol_type = mRNA
gccccgctccctacctaaggcgtgaggctacgagcggtcggctgtggcagcttctcttgtctctgacggcttgtagttatggggcaggagccgcggacgctgccgccctcccccaactggtactgcgcccgctgcagcgatgccgtgcccgggggcctctttggcttcgccgcgcggacctccgtcttccttgtccgcgtgggcccgggcgcaggcgagagtccagggacacccccgtttcgagtcataggagagttggtgggacacaccgaaagggtctctggcttcacattttctcatcaccctggtcagtacaacctctgtgccaccagctccgacgatgggactgtgaaaatatgggatgtagagacaaaaacagttgtgacagaacatgcactccatcagcatacgatatcaacattacattggtctcctcgagtaaaggacttaatagtatctggggatgaaaaaggagtagttttctgttactggtttaacagaaatgacagccagcacctctttatagaacccaggacaattttctgtcttacttgttcacctcatcatgaagatttagtagccattggctacaaggatggcatagtggtgataattgacatcagtaagaaaggagaagttattcataggcttcgaggccatgatgatgaaatccactccatagcctggtgtcccctgcctggtgaagattgtttatctataaaccaagaggaaacttcagaagaagctgaaattaccaacgggaatgctgtagcacaagctccagtaacaaaaggttgctacttagccactggaagcaaagatcaaaccattcgaatctggagctgttctagaggccgaggggtgatgattttgaaattgccctttctgaagagaagaggagggggtatagacccaactgttaaagagcgcctttggttgacactccattggcccagcaatcaaccaacacagctggtatctagctgttttggaggtgaactgttgcaatgggatctcactcaatcttggagacggaaatacaccctcttcagtgcctcatcagaagggcaaaatcattcaagaattgtgtttaatttatgtcctttacaaacagaggatgacaaacagctattactttctacatcaatggatagagatgtaaaatgttgggacatagccaccttggagtgcagctggacccttccttcccttggtgggtttgcatacagcctggctttctcttctgtggacataggctctttggccataggtgttggggatggcatgatccgtgtatggaatacactctccataaagaacaactatgatgtgaaaaatttttggcaaggcgtgaagtccaaggttacagcgctgtgctggcacccaaccaaggaaggttgcttagcttttggaactgatgatggaaaagtgggattgtatgacacctactccaacaagcctccacagatttctagcacatatcataagaagactgtatatactttagcctgggggccaccagtaccccccatgtcacttggaggagaaggagacagaccttcccttgctttatacagctgtggaggagaagggattgtcttacagcataatccctggaagcttagtggagaagcctttgacatcaacaaactcatcagggacaccaattcaatcaaatacaaattgcctgtacacacagagataagttggaaagcagatggcaaaatcatggctcttggcaatgaagatggatcaatagaaatatttcagattcccaacctgaaactgatctgtactatccaacagcatcacaagcttgtgaataccattagctggcatcatgagcatggcagccagccagaattgagctatctgatggcctctggctccaacaatgcagtcatttacgtgcacaacctgaagactgtcatagagagcagccctgagtctccagtgaccattacagagccctaccggaccctctcagggcatacggccaagattaccagtgtggcgtggagcccacatcatgatggaaggctggtatctgcttcctatgatggtacagcccaggtgtgggatgctctccgggaagagcccctgtgcaatttccgaggacatcgaggtcgactgctttgtgtggcatggtctcctttggatccagactgcatctattcaggggcagatgacttttgtgtgcacaagtggctcacttccatgcaagatcattcccggcctcctcaaggcaaaaaaagtattgaattggagaaaaaacggctctctcaacctaaggcaaagcccaaaaagaagaaaaagcccaccttgagaactcctgtaaagctggaatcgattgatggaaatgaagaagaaagcatgaaggagaactcaggacctgttgagaatggtgtgtcagaccaagaaggggaggagcaagcacgggagccggaattaccctgtggccttgctccagcggtttctagagaaccagttatctgcactccagtttcctcaggctttgaaaagtcaaaagtcaccattaataacaaagtcattttactgaaaaaggagccaccaaaagagaagccagaaaccttaatcaagaagagaaaagctcgttccttgcttcccctgagtacaagcctggaccacagatccaaagaggagcttcatcaggactgtttggtactagcaactgcaaagcactccagagagctgaatgaagatgtgtctgctgatgttgaggaaagatttcatctggggcttttcacagacagggctaccctgtatagaatgattgatattgaaggaaaaggtcacttagaaaatggccaccctgagttatttcaccagcttatgctttggaaaggagatctcaaaggtgttctccagactgcagcagaaagaggggagctgacagacaaccttgtggctatggcaccagcagctggctaccatgtgtggctatgggctgtggaagcttttgccaaacagctgtgttttcaggatcagtatgtcaaggctgcttctcacctactttccatccacaaagtgtatgaagcggtggagctgctcaagtcaaaccatttttacagggaagctattgcgattgccaaggcccggctgcgcccggaggacccagtcctgaaggacttgtacctcagctggggaaccgtcctagaaagagatggccactatgctgtagctgccaaatgctatttaggggccacttgtgcttatgatgcagccaaagttttggccaaaaagggggatgcggcatcacttagaacggctgcagagttggctgccatcgtaggagaggatgagttgtctgcttccctggctctcagatgtgcccaagagctgcttctggccaacaactgggtgggagcccaggaagccctgcagctgcatgaaagtctacagggtcagagattggtgttttgccttctggagctactgtccaggcatctggaggaaaagcagctttcagagggcaaaagctcctcctcttaccacacttggaacacgggcaccgaagggcctttcgtggagagggtgactgcagtgtggaagagcatcttcagccttgacacccctgagcagtatcaggaagcctttcagaagctgcagaacatcaagtacccatctgctacaaataacacacctgccaaacagctcctgcttcacatttgccatgacttgaccctggcagtgctgagccaacagatggcctcctgggacgaggctgtgcaggcgctccttcgggcggtggtccggagctatgactcagggagcttcaccatcatgcaggaagtgtactcagcctttctccctgacggctgtgaccacctaagagacaagttgggggaccatcaatcccctgccacaccagctttcaaaagtttggaggccttttttctttatgggcgtctgtatgaattctggtggtctctctccagaccttgcccaaattccagtgtctgggtaagggctggtcacagaacactctctgttgagccaagccagcagttagacactgccagcactgaagaaacggaccctgaaacttctcagccagagccaaacaggccttcagaactagacttgagactcacagaagaaggtgagcgaatgctgagtacttttaaggagctcttttcagaaaagcatgccagtctccaaaactcacagagaactgttgctgaagtccaagagaccttggcagaaatgatccgacaacaccaaaagagtcaactctgtaaatccacagcaaatggtcctgataagaatgaaccggaagtagaagcagagcagcccctctgcagttctcagagccagtgtaaagaagaaaaaaatgagccactttctctgcctgagttaaccaaaaggcttaccgaggcaaatcagagaatggcgaaatttcctgagagcattaaggcctggcccttcccagatgtgctggagtgctgcctcgtcctgcttctcatcaggtcccactttcctggctgtctggcccaggaaatgcagcagcaggcccaagagctccttcagaaatacggcaacacgaaaacttacagaagacactgccagaccttctgtatgtgaattttcacacaccttgaagaaactgccaaattgaaaatgtttgacatctttcacctctgcagttatgcctcaccagacattcactctggtccctagatgtttttgcagtaatccaaaagaatacaaacaaggattaagtttgaatcaaccctgcctacccatagacaacggtggatctgactttagactcaattgtggtctcctactggagggaagatcatgaaaagcccacagtagttattcagaactaacacctgcagagtgttggtcatctctacagccttaggcaggtttcacccaaagaggagaaacttctgtcgtcacccaaagtgttacatgcttaaaacacaagctacctttgtaaatacttcatctgatcagaagtgtgtcatgcttgtttgagatggagttgctgcattttaggactattgatacctttttttaattgtttttataatatttaatttgaaagaggagacccttctctctctactctttcatagactgaagtttgaatatgaaataggccttaaccatcatgttgactctcctgtcagaattttaggttggaaatttggttttattctttcatgtaattgcttatttgaacagatcacttactaaagctttagaagaagtgattcaaatgtgtgttttcccttcagttttataacaaatggattgatggcagtcaaatagctcaggaataaattactgtttcaatggttcttaaactttcttggatcataggatccttttgagaatcagattaaagccaaagatactctttggagaaaaa

SEQ ID NO: 2 - protein, 1508 aa, Homo sapiens
MGQEPRTLPPSPNWYCARCSDAVPGGLFGFAARTSVFLVRVGPGAGESPGTPPFRVIGELVGHTERVSGFTFSHHPGQYNLCATSSDDGTVKIWDVETKTVVTEHALHQHTISTLHWSPRVKDLIVSGDEKGVVFCYWFNRNDSQHLFIEPRTIFCLTCSPHHEDLVAIGYKDGIVVIIDISKKGEVIHRLRGHDDEIHSIAWCPLPGEDCLSINQEETSEEAEITNGNAVAQAPVTKGCYLATGSKDQTIRIWSCSRGRGVMILKLPFLKRRGGGIDPTVKERLWLTLHWPSNQPTQLVSSCFGGELLQWDLTQSWRRKYTLFSASSEGQNHSRIVFNLCPLQTEDDKQLLLSTSMDRDVKCWDIATLECSWTLPSLGGFAYSLAFSSVDIGSLAIGVGDGMIRVWNTLSIKNNYDVKNFWQGVKSKVTALCWHPTKEGCLAFGTDDGKVGLYDTYSNKPPQISSTYHKKTVYTLAWGPPVPPMSLGGEGDRPSLALYSCGGEGIVLQHNPWKLSGEAFDINKLIRDTNSIKYKLPVHTEISWKADGKIMALGNEDGSIEIFQIPNLKLICTIQQHHKLVNTISWHHEHGSQPELSYLMASGSNNAVIYVHNLKTVIESSPESPVTITEPYRTLSGHTAKITSVAWSPHHDGRLVSASYDGTAQVWDALREEPLCNFRGHRGRLLCVAWSPLDPDCIYSGADDFCVHKWLTSMQDHSRPPQGKKSIELEKKRLSQPKAKPKKKKKPTLRTPVKLESIDGNEEESMKENSGPVENGVSDQEGEEQAREPELPCGLAPAVSREPVICTPVSSGFEKSKVTINNKVILLKKEPPKEKPETLIKKRKARSLLPLSTSLDHRSKEELHQDCLVLATAKHSRELNEDVSADVEERFHLGLFTDRATLYRMIDIEGKGHLENGHPELFHQLMLWKGDLKGVLQTAAERGELTDNLVAMAPAAGYHVWLWAVEAFAKQLCFQDQYVKAASHLLSIHKVYEAVELLKSNHFYREAIAIAKARLRPEDPVLKDLYLSWGTVLERDGHYAVAAKCYLGATCAYDAAKVLAKKGDAASLRTAAELAAIVGEDELSASLALRCAQELLLANNWVGAQEALQLHESLQGQRLVFCLLELLSRHLEEKQLSEGKSSSSYHTWNTGTEGPFVERVTAVWKSIFSLDTPEQYQEAFQKLQNIKYPSATNNTPAKQLLLHICHDLTLAVLSQQMASWDEAVQALLRAVVRSYDSGSFTIMQEVYSAFLPDGCDHLRDKLGDHQSPATPAFKSLEAFFLYGRLYEFWWSLSRPCPNSSVWVRAGHRTLSVEPSQQLDTASTEETDPETSQPEPNRPSELDLRLTEEGERMLSTFKELFSEKHASLQNSQRTVAEVQETLAEMIRQHQKSQLCKSTANGPDKNEPEVEAEQPLCSSQSQCKEEKNEPLSLPELTKRLTEANQRMAKFPESIKAWPFPDVLECCLVLLLIRSHFPGCLAQEMQQQAQELLQKYGNTKTYRRHCQTFCM

SEQ ID NO: 3 - RNA, 1482 nt, Homo sapiens
  source 1..1482: mol_type = mRNA
gcacccgcgggtttgctatggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacaaaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggtttcagacaaaatcaaaaagaaggaaggtgctcacattccttaaattaaggagaaatgctggcatagagcagcactaaatgacaccactaaagaaacgatcagacagatctggaatgtgaagcgttatagaagataactggcctcatttcttcaaaatatcaagtgttgggaaagaaaaaaggaagtggaatgggtaactcttcttgattaaaagttatgtaataaccaaatgcaatgtgaaatattttactggactctattttgaaaaaccatctgtaaaagactggggtgggggtgggaggccagcacggtggtgaggcagttgagaaaatttgaatgtggattagattttgaatgatattggataattattggtaattttatgagctgtgagaagggtgttgtagtttataaaagactgtcttaatttgcatacttaagcatttaggaatgaagtgttagagtgtcttaaaatgtttcaaatggtttaacaaaatgtatgtgaggcgtatgtggcaaaatgttacagaatctaactggtggacatggctgttcattgtactgtttttttctatcttctatatgtttaaaagtatataataaaaatatttaatttttttttaaatta

SEQ ID NO: 4 - RNA, 1482 nt, Homo sapiens
  source 1..1482: mol_type = mRNA
gcacccgcgggtttgctatggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacaaaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggttttagacaaaatcaaaaagaaggaaggtgctcacattccttaaattaaggagaaatgctggcatagagcagcactaaatgacaccactaaagaaacgatcagacagatctggaatgtgaagcgttatagaagataactggcctcatttcttcaaaatatcaagtgttgggaaagaaaaaaggaagtggaatgggtaactcttcttgattaaaagttatgtaataaccaaatgcaatgtgaaatattttactggactctattttgaaaaaccatctgtaaaagactgaggtgggggtgggaggccagcacggtggtgaggcagttgagaaaatttgaatgtggattagattttgaatgatattggataattattggtaattttatgagctgtgagaagggtgttgtagtttataaaagactgtcttaatttgcatacttaagcatttaggaatgaagtgttagagtgtcttaaaatgtttcaaatggtttaacaaaatgtatgtgaggcgtatgtggcaaaatgttacagaatctaactggtggacatggctgttcattgtactgtttttttctatcttctatatgtttaaaagtatataataaaaatatttaatttttttttaaatta

SEQ ID NO: 5 - DNA, 18 bp, synthetic construct
  source 1..18: mol_type = other DNA
tcactttcataatgctgg

SEQ ID NO: 6 - DNA, 15 bp, synthetic construct
  source 1..15: mol_type = other DNA
tttcataatgctggc

SEQ ID NO: 7 - DNA, 15 bp, synthetic construct
  source 1..15: mol_type = other DNA
tgctggcagacttac

SEQ ID NO: 8 - DNA, 15 bp, synthetic construct
  source 1..15: mol_type = other DNA
cataatgctggcaga

SEQ ID NO: 9 - DNA, 15 bp, synthetic construct
  source 1..15: mol_type = other DNA
tcataatgctggcag

SEQ ID NO: 10 - DNA, 15 bp, synthetic construct
  source 1..15: mol_type = other DNA
ttcataatgctggca

SEQ ID NO: 11 - DNA, 15 bp, synthetic construct
  source 1..15: mol_type = other DNA
tttcataatgctggc

SEQ ID NO: 12 - DNA, 20 bp, synthetic construct
  source 1..20: mol_type = other DNA
attcactttcataatgctgg

SEQ ID NO: 13 - DNA, 18 bp, synthetic construct
  source 1..18: mol_type = other DNA
tcactttcataatgctgg

SEQ ID NO: 14 - DNA, 15 bp, synthetic construct
  source 1..15: mol_type = other DNA
ctttcataatgctgg

SEQ ID NO: 15 - DNA, 12 bp, synthetic construct
  source 1..12: mol_type = other DNA
tcataatgctgg

SEQ ID NO: 16 - DNA, 15 bp, synthetic construct
  source 1..15: mol_type = other DNA
actttcataatgctg

SEQ ID NO: 17 - DNA, 12 bp, synthetic construct
  source 1..12: mol_type = other DNA
ttcataatgctg

SEQ ID NO: 18 - DNA, 15 bp, synthetic construct
  source 1..15: mol_type = other DNA
cactttcataatgct

SEQ ID NO: 19 - DNA, 12 bp, synthetic construct
  source 1..12: mol_type = other DNA
tttcataatgct

SEQ ID NO: 20 - DNA, 15 bp, synthetic construct
  source 1..15: mol_type = other DNA
tcactttcataatgc

SEQ ID NO: 21 - DNA, 12 bp, synthetic construct
  source 1..12: mol_type = other DNA
ctttcataatgc

SEQ ID NO: 22 - DNA, 15 bp, synthetic construct
  source 1..15: mol_type = other DNA
ttcactttcataatg

SEQ ID NO: 23 - DNA, 12 bp, synthetic construct
  source 1..12: mol_type = other DNA
actttcataatg

SEQ ID NO: 24 - DNA, 15 bp, synthetic construct
  source 1..15: mol_type = other DNA
attcactttcataat

SEQ ID NO: 25 - DNA, 12 bp, synthetic construct
  source 1..12: mol_type = other DNA
cactttcataat

SEQ ID NO: 26 - DNA, 15 bp, synthetic construct
  source 1..15: mol_type = other DNA
gattcactttcataa

SEQ ID NO: 27 - DNA, 12 bp, synthetic construct
  source 1..12: mol_type = other DNA
tcactttcataa

SEQ ID NO: 28 - DNA, 12 bp, synthetic construct
  source 1..12: mol_type = other DNA
ttcactttcata

SEQ ID NO: 29 - DNA, 12 bp, synthetic construct
  source 1..12: mol_type = other DNA
attcactttcat

SEQ ID NO: 30 - DNA, 15 bp, synthetic construct
  source 1..15: mol_type = other DNA
agtaagattcacttt

SEQ ID NO: 31 - RNA, 18 nt, synthetic construct
  source 1..18: mol_type = other RNA
  modified_base 1: mod_base = OTHER; note = methyl group at 5-position
  modified_base 2: mod_base = OTHER; note = methyl group at 5-position
  modified_base 4: mod_base = OTHER; note = methyl group at 5-position
  modified_base 5: mod_base = OTHER; note = methyl group at 5-position
  modified_base 6: mod_base = OTHER; note = methyl group at 5-position
  modified_base 7: mod_base = OTHER; note = methyl group at 5-position
  modified_base 8: mod_base = OTHER; note = methyl group at 5-position
  modified_base 10: mod_base = OTHER; note = methyl group at 5-position
  modified_base 13: mod_base = OTHER; note = methyl group at 5-position
  modified_base 15: mod_base = OTHER; note = methyl group at 5-position
  modified_base 16: mod_base = OTHER; note = methyl group at 5-position
tcactttcataatgctgg

SEQ ID NO: 32 - RNA, 20 nt, synthetic construct
  source 1..20: mol_type = other RNA
attcactttcataatgctgg

SEQ ID NO: 33 - RNA, 10 nt, synthetic construct
  source 1..10: mol_type = other RNA
ttgcctttct